(19) United States
(12) Patent Application Publication
(10) Pub. No.: US 2016/0045654 A1
(43) Pub. Date: Feb. 18, 2016

(54) Implanted Extracardiac Device for Circulatory Assistance

(71) Applicant: Robert A. Connor, Forest Lake, MN (US)

(72) Inventor: Robert A. Connor, Forest Lake, MN (US)

(73) Assignee: Medibotics LLC, Forest Lake, MN (US)

(21) Appl. No.: 14/459,937

(22) Filed: Aug. 14, 2014

(51) Int. Cl. A61M 1/12; A61F 2/06; A61F 2/82; A61M 1/10

(52) U.S. Cl. CPC A61M 1/125; A61M 1/122; A61M 1/1008; A61F 2/064; A61F 2/82; A61M 1/1072; A61M 1/1015; A61M 1/1005; A61M 1/1081; A61M 1/1039; A61M 1/1084; A61M 1/1086; A61M 1/127; A61M 2205/04; A61F 2002/061; A61F 2220/0016; A61M 2205/0266; A61M 2205/0294; A61M 2205/3303; A61M 2230/10; A61M 2230/04; A61M 2230/205; A61M 2230/63; A61M 2230/65; A61M 2230/50; A61M 2205/3365; A61M 2230/201; A61M 2230/432; A61M 2205/3306; A61M 2205/3317; A61M 2230/208

(57) ABSTRACT

This invention is an implanted extracardiac device for supplementing blood circulation which comprises an implanted blood flow lumen, a blood flow increasing mechanism, and a control unit. Its design improves blood circulation when the blood flow increasing mechanism is operating, without hindering native blood flow when the mechanism is not operating. This device improves circulation without intruding on cardiac tissue or weakening the heart by completely supplanting cardiac function. Also, since the device allows native blood flow when the blood flow increasing mechanism is not in operation, it requires less power and can enable more patient mobility.

### CROSS-REFERENCE TO RELATED APPLICATIONS

This patent application claims the priority benefit of U.S. Provisional Patent Application No. 61/866,583 by Robert A. Connor entitled “Stent for Actively Accelerating Blood Flow” filed on Aug. 16, 2013, the entire contents of which is incorporated herein by reference.

### FEDERALLY SPONSORED RESEARCH

Not Applicable

### SEQUENCE LISTING OR PROGRAM

Not Applicable

### BACKGROUND

### Field of Invention

This invention relates to cardiac function and blood circulation.

### Introduction to Heart Failure

Proper blood circulation throughout the body is essential to provide oxygen and nutrients to body tissue, as well as to remove waste products. Impairment of blood circulation can result in tissue death and loss of organ function. As the central pumping mechanism of the body's circulatory system, the heart is central to ensuring proper blood circulation. Heart failure is the inability of the heart to continue to provide consistent and sufficient blood flow to meet the body's needs. Congestive Heart Failure (CHF) is a chronic condition which is characterized by progressive deterioration of the heart's ability to provide consistent and sufficient blood flow to meet the body's needs. Heart failure can be aggravated by long-term factors such as decreased elasticity in blood vessels. Heart failure can also be acutely triggered or exacerbated by specific adverse events such as Acute Myocardial Infarction (AMI). In Congestive Heart Failure (CHF), cardiac muscle weakens, cardiac output decreases, blood circulates at a slower rate, intracardiac pressure increases, and blood circulation becomes inadequate.

Congestive Heart Failure (CHF) is a serious, prevalent, and growing condition. The costs of CHF are very large in terms of human mortality and suffering, as well as dollars. CHF affects millions of people worldwide. Hundreds of thousands die from CHF complications each year. CHF is the leading cause of hospitalization for people over the age of 65 in the U.S. Further, the prevalence of CHF has grown dramatically during the past two decades. For people in the most severe stages of CHF, wait times for heart transplantation can exceed 2-3 years with significant mortality rates during the wait. There are currently some pharmacological, medical device, and surgical approaches to address CHF, but they all have limitations. None are universally available and effective for the millions of people with CHF.

### REVIEW AND LIMITATIONS OF THE PRIOR ART

Pharmacological approaches include ACE inhibitors, beta blockers, and diuretics. These drugs are useful options for first line therapy, but their limitations include patient non-compliance, hypotension, potential interference with the body's natural compensatory mechanisms, non-suitability for emergency use, and insufficient therapeutic effect for patients in severe stage CHF. Cardiac Resynchronization Therapy (CRT) is a medical device approach based on cardiac pacing. It can also be a useful option for CHF, but there is a large percentage of people with CHF who are unresponsive to CRT and chronic high-rate pacing can have adverse effects on some people. Intra-Aortic Balloon Pump (IABP) therapy can help to reduce the heart's workload for people in severe stage CHF, but IABP therapy can restrict patient ambulation, is not well suited for long-term use, and can decrease Mean Arterial Pressure (MAP) for some organs.

Left Ventricular Assist Device (LVAD) therapy comprises using a mechanical pump to partially or completely replace the pumping function of the left ventricle of the heart. LVAD therapy can be useful for people in severe stage CHF, especially as a bridge to heart transplantation, but it also has limitations. These limitations include: intrusion into heart tissue which can further traumatize an already-weakened heart and decrease the chances for recovery (apart from a heart transplant), significant power required for constant cardiac-level pumping and the associated restrictions on patient ambulation, inability to focus circulatory benefits for a particular body organ that is in greatest need, and significant mortality rates for people waiting for scarce heart transplants. Heart transplantation can be effective for people with severe stage CHF, but there are long wait times for available hearts, the operation itself can be risky, and transplantation is too extreme and invasive to appropriately help people in earlier stage CHF. Other approaches to addressing CHF include mechanical removal of fluid from blood, but are not well-suited for everyone with CHF.

Recent prior art also includes some innovative patents for peripheral vessel blood pumps which operate at sub-cardiac rates and for a device which incorporates a blood pump into a stent. Examples of this prior art include U.S. Pat. No. 7,905,823 (Farnan et al., Mar. 15, 2011, “Devices, Methods and Systems for Establishing Supplemental Blood Flow in the Circulatory System”), U.S. Pat. No. 7,998,190 (Gharib et al., Aug. 16, 2011, “Intravascular Miniature Stent Pump”), U.S. Pat. No. 8,157,720 (Marseille et al., Apr. 17, 2012, “Heart Assist System”), U.S. Pat. No. 8,465,410 (Marseille et al., Jun. 18, 2013, “Heart Assist System”), U.S. Pat. No. 8,545,380 (Farnan et al., Oct. 1, 2013, “Intravascular Blood Pump and Catheter”), and U.S. Pat. No. 8,768,487 (Farnan et al., Jul. 1, 2014, “Devices, Methods and Systems for Establishing Supplemental Blood Flow in the Circulatory System”).

Innovative examples of this type of prior art also include U.S. Patent Applications 20080076959 (Farnan et al., Mar. 27, 2008, “Devices, Methods and Systems for Establishing Supplemental Blood Flow in the Circulatory System”), 20090171137 (Farnan et al., Jul. 2, 2009, “Intravascular Blood Pump and Catheter”), 20090182188 (Marseille et al., Jul. 16, 2009, “Devices, Methods and Systems for Establishing Supplemental Blood Flow in the Circulatory System”), 20110112353 (Farnan et al., May 12, 2011, “Bifurcated Outflow Cannulae”), 20110137234 (Farnan et al., Jun. 9, 2011, “Methods for Establishing Supplemental Blood Flow in the Circulatory System”), 20110196190 (Farnan et al., Aug. 11, 2011, “Devices, Methods and Systems for Establishing Supplemental Blood Flow in the Circulatory System”), 20140005467 (Farnan et al., Jan. 2, 2014, “Intravascular Blood Pump and Catheter”), and 20140073837 (Kerkhoffs et al., Mar. 13, 2014, “Blood Flow System with Variable Speed Control”).

However, even with these recent innovative examples in the prior art, there are still device design challenges which have not been fully resolved. For example, how can one design a supplemental extracardiac blood flow increasing device which accelerates blood flow when it is in operation without hindering native blood flow when it is not in operation? How can one design a supplemental extracardiac blood flow increasing device which bifurcates blood flow without inducing thrombogenesis? How can one design a supplemental extracardiac blood flow increasing device which selectively directs improved circulation to those body organs which are in greatest need? How can the operation of a supplemental extracardiac blood flow increasing device be informed by data from implanted or wearable sensors in order to optimally reduce heart workload without supplanting cardiac function in a manner that reduces the chances for healing and recovery? These are some of the unresolved design challenges which are addressed by the invention disclosed herein. Hopefully this invention will provide a novel and useful addition to treatment options for this serious, prevalent, and growing circulatory condition.

### SUMMARY AND ADVANTAGES OF THIS INVENTION

The Hippocratic Oath enjoins health care providers to “Do no harm.” This injunction also applies to this invention. The purpose of this present invention is to reduce cardiac workload and improve blood circulation while avoiding some of the negative side effects which can occur with approaches in the prior art. For example, this invention is embodied in a device which is implanted outside the heart so that it does not potentially traumatize already-weakened heart tissue. This can help to allow cardiac healing and to maintain the possibility that the heart will recover and transplantation will not be needed. As another example, this device is designed to avoid hindering native blood flow when a blood flow increasing mechanism (such as a blood pump) is not in operation. Accordingly, this device does not have to operate all the time. This reduces power requirements and can also reduce the possibility of adverse outcomes in the event of unexpected power failure. It also can free a person to be ambulatory and have a higher quality of life. The goal of this invention is to create a truly-supplemental extracardiac circulatory assistance device which achieves improved circulation with reasonable power requirements, without undermining the possibility of cardiac healing and recovery.

More specifically, this invention can be embodied in an implanted device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

In an example, a pre-implantation minimum cross-sectional flow area can be defined as the minimum cross-sectional flow area from the upstream location to the downstream location in a blood vessel before the implanted blood flow lumen and the blood flow increasing mechanism are implanted into fluid communication with the blood vessel. Also, a post-implantation minimum cross-sectional flow area can be defined as the minimum cross-sectional flow area from the upstream location to the downstream location which is unobstructed by the blood flow increasing mechanism when the blood flow increasing mechanism is not in operation after the implanted blood flow lumen and the blood flow increasing mechanism are implanted. In an example, this device can be designed so that the post-implantation minimum cross-sectional flow area is not substantially less than the pre-implantation minimum cross-sectional flow area.

Expressing this in terms of blood flow rates, post-implantation blood flow rate is greater than pre-implantation blood flow when a blood flow increasing mechanism is in operation transducing electromagnetic energy into kinetic energy. Further, and more innovative, post-implantation blood flow rate is not substantially less than pre-implantation blood flow rate when the blood flow increasing mechanism is not in operation. In an example, the definition of substantially less can selected from: 5% less, 10% less, and 25% less.

Potential advantages of this invention over various approaches in the prior art include the following. First, this device can improve blood circulation when a blood flow increasing mechanism is in operation (transducing electromagnetic energy into blood flow) without hindering native blood flow when the blood flow increasing mechanism is not in operation. Second, this device can improve circulation without harming cardiac tissue by intrusion or further weakening the heart by completely supplanting its function. Third, the ability of this device to allow native blood flow when a blood flow increasing mechanism is not in operation can help to reduce its power requirements, free a person with CHF to be ambulatory, and reduce the possibility of adverse outcomes if there is an unexpected loss of power.

In a more-general example, a plurality of these devices can be implanted in a distributed manner in different peripheral blood vessels. This can create a system of distributed supplemental circulatory assistance which reduces cardiac workload until the heart recovers or for the long-term if recovery does not occur. Such a system of distributed supplemental circulatory assistance can also selectively direct the greatest improvements in blood circulation toward those organs with the greatest need (such as the kidneys).

### BRIEF DESCRIPTION OF THE SEVERAL VIEWS OF THE DRAWINGS

FIGS. 1 through 98 show examples of how this invention can be embodied, but they do not limit the full generalizability of the claims.

FIGS. 1 and 2 show a stent with a pump with an axis that is perpendicular to the stent.

FIGS. 3 and 4 show a stent with a pump with an axis that is perpendicular to the stent with electromagnetically-driven rotary pump.

FIGS. 5 and 6 show a stent with a pump entirely within a blood vessel.

FIGS. 7 and 8 show a stent with a pump outside a blood vessel.

FIGS. 9 and 10 show a stent with a pump with an axis that is parallel to the stent.

FIGS. 11 and 12 show a stent with a pump with an axis that is parallel to the stent that is entirely within a blood vessel.

FIGS. 13 through 15 show an implanted blood flow lumen with a pump with an axis that is perpendicular to the lumen.

FIGS. 16 through 18 show an implanted blood flow lumen with a pump with an axis that is parallel to the lumen.

FIGS. 19 through 21 show an implanted blood flow lumen with a peristaltic pump.

FIGS. 22 through 24 show an implanted blood flow lumen with a compressive member and one-way valves.

FIGS. 25 through 27 show an implanted blood flow lumen with an electromagnetic field flow drive.

FIGS. 28 through 30 show an implanted blood flow lumen with an electromagnetically-driven rotary pump.

FIGS. 31 through 33 show an implanted blood flow lumen with a longitudinal membrane wave pump.

FIGS. 34 through 36 show an implanted blood flow lumen with a pump with an axis that is perpendicular to the lumen with the addition of three-way connectors.

FIGS. 37 through 39 show an implanted blood flow lumen with a pump with an axis that is parallel to the lumen with the addition of three-way connectors.

FIGS. 40 through 42 show an implanted blood flow lumen with a peristaltic pump with the addition of three-way connectors.

FIGS. 43 through 45 show an implanted blood flow lumen with a compressive member and one-way valves with the addition of three-way connectors.

FIGS. 46 through 48 show an implanted blood flow lumen with an electromagnetic field flow drive with the addition of three-way connectors.

FIGS. 49 through 51 show an implanted blood flow lumen with an electromagnetically-driven rotary pump with the addition of three-way connectors.

FIGS. 52 through 54 show an implanted blood flow lumen with a longitudinal membrane wave pump with the addition of three-way connectors.

FIGS. 55 through 57 show an implanted blood flow lumen with a pump with an axis that is perpendicular to the lumen, wherein the lumen replaces a vessel segment.

FIGS. 58 through 60 show an implanted blood flow lumen with a pump with an axis that is parallel to the lumen, wherein the lumen replaces a vessel segment.

FIGS. 61 through 63 show an implanted blood flow lumen with a peristaltic pump, wherein the lumen replaces a vessel segment.

FIGS. 64 through 66 show an implanted blood flow lumen with a compressive member and one-way valves, wherein the lumen replaces a vessel segment.

FIGS. 67 through 69 show an implanted blood flow lumen with an electromagnetic field flow drive, wherein the lumen replaces a vessel segment.

FIGS. 70 through 72 show an implanted blood flow lumen with an electromagnetically-driven rotary pump, wherein the lumen replaces a vessel segment.

FIGS. 73 through 75 show an implanted blood flow lumen with a longitudinal membrane wave pump, wherein the lumen replaces a vessel segment.

FIGS. 76 through 79 show an implanted device for adjustment of blood pressure level or blood pressure variation.

FIGS. 80 through 82 show a bulbous implanted blood flow lumen with two rotary pumps and three blood flow channels.

FIGS. 83 through 85 show a bulbous implanted blood flow lumen with a centrally-suspended rotary pump.

FIGS. 86 through 88 show an implanted blood flow lumen with a retractable rotary pump.

FIGS. 89 through 91 show an implanted blood flow lumen and pump with centrally-extendable fins which comprise an impeller.

FIGS. 92 through 95 show an implanted blood flow lumen with two crankshaft-like rotating members.

FIGS. 96 through 98 show an implanted blood flow lumen and pump with twistable strips which comprise an impeller.

### DETAILED DESCRIPTION OF THE FIGURES

Before we discuss the specific examples shown in the figures, it is worthwhile to provide an introductory discussion which defines some important terms, introduces some important design characteristics, and outlines some of the alternative configurations which will appear in multiple figures. As noted above, this invention can be embodied in an implanted device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

In an example, a pre-implantation minimum cross-sectional flow area can be defined as the minimum cross-sectional flow area from the upstream location to the downstream location in a blood vessel before the implanted blood flow lumen and the blood flow increasing mechanism are implanted. Also, a post-implantation minimum cross-sectional flow area can be defined as the minimum cross-sectional flow area from the upstream location to the downstream location which is unobstructed by the blood flow increasing mechanism when the blood flow increasing mechanism is not in operation after the implanted blood flow lumen and the blood flow increasing mechanism are implanted. In an example, this device can be designed so that the post-implantation minimum cross-sectional flow area is not substantially less than the pre-implantation minimum cross-sectional flow area. In terms of flow rates, post-implantation blood flow rate is greater than pre-implantation blood flow when the blood flow increasing mechanism is in operation transducing electromagnetic energy into kinetic energy. Further, post-implantation blood flow rate is not substantially less than pre-implantation blood flow rate when the blood flow increasing mechanism is not in operation. In an example, the definition of “substantially less” can be selected from the group consisting of: 5% less, 10% less, and 25% less.

We now turn our attention to the implanted blood flow lumen (which can be a stent or artificial blood vessel) and the implanted blood flow increasing mechanism (which can be a blood pump) which are configured to be implanted so as to be in fluid communication with the interior of a blood vessel. The following are some issues with respect to alternative configurations for the implanted blood flow lumen and the blood flow increasing mechanism. In an example, the implanted blood flow lumen and the blood flow increasing mechanism can both be configured to be implanted entirely within the walls of a natural blood vessel in an “internal vessel” approach. An advantage of this internal-vessel approach is that this device can be implanted in a minimally invasive manner—ideally implanted in an endovascular and/or transluminal manner. Another advantage of this approach is that it avoids bifurcating blood flows which can be thrombogenic. A potential disadvantage of this approach is that the blood flow increasing mechanism (especially if it is a blood pump with an impeller) can obstruct the natural cross-sectional area of the blood vessel and hinder native blood flow when the blood flow increasing mechanism is turned off and/or loses power. The resulting need for constant operation of a pump can cause high power requirements and restrict patient ambulation. Herein, we disclose novel device designs and methods to gain the advantages of this internal-vessel approach (e.g. endovascular implantation) while minimizing the disadvantages (e g minimal or no restriction of native flow when a pump is not operating).

In another example, an implanted blood flow lumen can be configured to be implanted at least partially outside the walls of the natural blood vessel with which the implanted blood flow lumen is in fluid communication. In an example, an implanted blood flow lumen can bifurcate (and then reconverge) blood flow from an upstream location to a downstream location. In an example, an implanted blood flow lumen can divide pre-implantation blood flow through a natural blood vessel from an upstream location to a downstream location into a first blood flow and a second blood flow. In an example, these two blood flows can flow in parallel (in terms of flow dynamics even if not parallel in terms of geometry) for a while. In an example, these first and second flows can diverge at an upstream location and then reconverge at a downstream location.

In an example, an implanted blood flow increasing mechanism can be an extracardiac blood pump. In an example, an implanted blood flow increasing mechanism can be configured to be in fluid communication with a first blood flow, with a second blood flow, or with both first and second flows. In an example, an implanted blood flow increasing mechanism can increase the flow of blood through the implanted blood flow lumen, through the natural blood vessel, or both. In an example, the blood flow increasing mechanism can increase the rate of blood flow from the upstream location to the downstream location. An advantage of implanting an implanted blood flow lumen at least partially outside the walls of a natural blood vessel is that this provides additional space to create a greater cross-sectional flow area through which blood can flow in the combination of the implanted blood flow lumen and the natural blood vessel. A potential disadvantage of this approach is that it requires at least some disruption of the natural blood vessel walls. Also, it must be designed to minimize thrombogenesis at blood flow junctures.

In another example, an implanted blood flow lumen can be configured to be spliced into a natural blood vessel (from an upstream location to a downstream location) so as to entirely replace a longitudinal segment of the natural blood vessel. With respect to flow dynamics, in this case blood flow through the natural blood vessel and blood flow through the implanted blood flow lumen are in series, not in parallel. An advantage of this splicing approach is that blood flow need not be bifurcated; this can reduce potential thrombogenesis from flow junctures. Even when blood flows are divided among multiple intra-luminal channels within an implanted blood flow lumen, there is greater design flexibility in an entirely-manufactured blood flow lumen. This design flexibility can be used to create hemodynamic flow patterns which minimize thrombogenesis despite the splitting of blood flows. A potential disadvantage of this splicing approach is that it involves the removal of a longitudinal segment of the natural blood vessel, which is more invasive than some other approaches.

In an example, an implanted blood flow lumen can be configured to be implanted into fluid communication with a natural blood vessel by one or more connecting members or connection methods selected from the group consisting of: endovascular and/or transluminal insertion and expansion, surgical anastomosis, surgical sutures, purse string suture, drawstring, pull tie, friction fit, surgical staples, tissue adhesive, gel, fluid seal, chemical bonding, cauterization, blood vessel connector and/or joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

In an example, an implanted blood flow lumen can be selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber. In an example, an implanted blood flow lumen can have a longitudinal axis which is relatively straight. In an example, an implanted blood flow lumen can have a longitudinal axis which is arcuate. In an example, an implanted blood flow lumen can have a longitudinal axis which follows the shape of longitudinal axis of the natural blood vessel with which the implanted blood flow lumen is in fluid communication.

In an example, an implanted blood flow lumen can have a single interior channel through which blood flows. In an example, an implanted blood flow lumen can have multiple interior flow channels into which incoming blood flow is separated into different sub-flows. In an example, multiple interior flow channels can reconverge at a downstream location within the implanted blood flow lumen. In an example, multiple interior flow channels can be substantially parallel. In an example, an implanted blood flow lumen can comprise one or more branches. In an example, an implanted blood flow lumen can comprise two or more inflow branches which converge into one outflow lumen. In an example, an implanted blood flow lumen can comprise one inflow lumen which diverges into two or more outflow branches.

In an example, an implanted blood flow lumen can have a substantially uniform cross-sectional shape along the entire length of its longitudinal axis. In an example, an implanted blood flow lumen can have a non-uniform cross-sectional shape along its longitudinal axis. In an example, an implanted blood flow lumen can be tapered. In an example, an implanted blood flow lumen can be bulbous. In an example, an implanted blood flow lumen can have a substantially circular cross-sectional shape. In an example, an implanted blood flow lumen can have a conic section cross-sectional shape. In an example, an implanted blood flow lumen can have an ovaloid or elliptical cross-sectional shape. In an example, an implanted blood flow lumen can have a square or other polygonal cross-sectional shape. In an example, an implanted blood flow lumen can have a cross-sectional shape which is composed of multiple circles or polygons.

In an example, an implanted blood flow lumen can be manufactured in an inorganic manner and/or from non-biological materials. In an example, an implanted blood flow lumen can be created using biological processes and/or from biological materials. In an example, an implanted blood flow lumen can be created by growing biological tissue on a scaffold. In an example, an implanted blood flow lumen can be an artificial vessel segment or branch. In an example, an implanted blood flow lumen can be a natural vessel segment or branch which is transplanted. In an example, the elasticity of an implanted blood flow lumen can be substantially the same as that of a natural blood vessel. In an example, the elasticity of an implanted blood flow lumen can be greater than that of a natural blood vessel in order to reduce cardiac workload. In an example, an implanted flow lumen can further comprise an elastic-walled blood reservoir. In an example, the elasticity of an implanted blood flow lumen can be less than that of a natural blood vessel in order to better control hemodynamics.

In an example, the cross-sectional flow area of an implanted blood flow lumen can be substantially the same as the cross-sectional flow area of the pre-implantation interior of the natural blood vessel with which the implanted blood flow lumen is connected. In an example, the average cross-sectional flow area of an implanted blood flow lumen (averaged along its longitudinal axis) can be substantially the same as the average cross-sectional flow area of the pre-implantation interior of the natural blood vessel (averaged along its longitudinal axis) with which the implanted blood flow lumen is connected. In an example, the minimum cross-sectional flow area of an implanted blood flow lumen (along its longitudinal axis) can be substantially the same as the minimum cross-sectional flow area of the pre-implantation interior of the natural blood vessel (along its longitudinal axis) with which the implanted blood flow lumen is connected.

In an example, the cross-sectional flow area of an implanted blood flow lumen is not substantially less than the cross-sectional flow area of the pre-implantation interior of the natural blood vessel with which the implanted blood flow lumen is connected. In an example, the average cross-sectional flow area of an implanted blood flow lumen (averaged along its longitudinal axis) is not substantially less than same as the average cross-sectional flow area of the pre-implantation interior of the natural blood vessel (averaged along its longitudinal axis) with which the implanted blood flow lumen is connected. In an example, the minimum cross-sectional flow area of an implanted blood flow lumen (along its longitudinal axis) is not substantially less than the minimum cross-sectional flow area of the pre-implantation interior of the natural blood vessel (along its longitudinal axis) with which the implanted blood flow lumen is connected. In an example, the definition of substantially less can be selected from the group consisting of: 5% less, 10% less, and 25% less.

In an example, the cross-sectional flow area of an implanted blood flow lumen can be substantially greater than the cross-sectional flow area of the pre-implantation interior of the natural blood vessel with which the implanted blood flow lumen is connected. In an example, the average cross-sectional flow area of an implanted blood flow lumen (averaged along its longitudinal axis) can be greater than the average cross-sectional flow area of the pre-implantation interior of the natural blood vessel (averaged along its longitudinal axis) with which the implanted blood flow lumen is connected. In an example, the minimum cross-sectional flow area of an implanted blood flow lumen (along its longitudinal axis) can be greater than the minimum cross-sectional flow area of the pre-implantation interior of the natural blood vessel (along its longitudinal axis) with which the implanted blood flow lumen is connected. In an example, the definition of substantially greater can be selected from the group consisting of: 5% more, 25% more, 50% more, and 100% more.

In an example, the gross cross-sectional flow area of an implanted blood flow lumen can be defined as the interior cross-sectional area of that lumen without considering any cross-sectional flow obstruction by the impellor (or other parts) of a blood flow increasing mechanism which is in fluid communication with the interior of that blood flow lumen. In an example, the net cross-sectional flow area of an implanted blood flow lumen can be defined as the interior cross-sectional area of that lumen which remains after subtracting out the cross-sectional flow area which is obstructed by the impellor (or other parts) of a blood flow increasing mechanism.

In an example, the net cross-sectional flow area of an implanted blood flow lumen can be substantially the same as the cross-sectional flow area of the pre-implantation interior of the natural blood vessel with which the implanted blood flow lumen is connected. In an example, the average net cross-sectional flow area of an implanted blood flow lumen (averaged along its longitudinal axis) can be substantially the same as the average cross-sectional flow area of the pre-implantation interior of the natural blood vessel (averaged along its longitudinal axis) with which the implanted blood flow lumen is connected. In an example, the minimum net cross-sectional flow area of an implanted blood flow lumen (along its longitudinal axis) can be substantially the same as the minimum cross-sectional flow area of the pre-implantation interior of the natural blood vessel (along its longitudinal axis) with which the implanted blood flow lumen is connected.

In an example, the net cross-sectional flow area of an implanted blood flow lumen is not substantially less than the cross-sectional flow area of the pre-implantation interior of the natural blood vessel with which the implanted blood flow lumen is connected. In an example, the average net cross-sectional flow area of an implanted blood flow lumen (averaged along its longitudinal axis) is not substantially less than the same as the average cross-sectional flow area of the pre-implantation interior of the natural blood vessel (averaged along its longitudinal axis) with which the implanted blood flow lumen is connected. In an example, the minimum net cross-sectional flow area of an implanted blood flow lumen (along its longitudinal axis) is not substantially less than the same as the minimum cross-sectional flow area of the pre-implantation interior of the natural blood vessel (along its longitudinal axis) with which the implanted blood flow lumen is connected.

In an example, the net cross-sectional flow area of an implanted blood flow lumen can be greater than the cross-sectional flow area of the pre-implantation interior of the natural blood vessel with which the implanted blood flow lumen is connected. In an example, the average net cross-sectional flow area of an implanted blood flow lumen (averaged along its longitudinal axis) can be greater than the average cross-sectional flow area of the pre-implantation interior of the natural blood vessel (averaged along its longitudinal axis) with which the implanted blood flow lumen is connected. In an example, the minimum net cross-sectional flow area of an implanted blood flow lumen (along its longitudinal axis) can be greater than the minimum cross-sectional flow area of the pre-implantation interior of the natural blood vessel (along its longitudinal axis) with which the implanted blood flow lumen is connected.

In an example, the amount by which a blood flow increasing mechanism obstructs the cross-sectional flow area of an implanted blood flow lumen can change when the blood flow increasing member starts to operate. In an example, a blood flow increasing member can have a first configuration with a first amount of obstruction of the cross-sectional flow area of an implanted blood flow lumen and a second configuration with a second amount of obstruction of the cross-sectional flow area of an implanted blood flow lumen. In an example, the second amount can be substantially greater the first amount. In an example, substantially greater can be at least 10% greater. In an example, substantially greater can be at least 25% greater. In an example, substantially greater can be at least 50% greater. In an example, substantially greater can be at least 100% greater.

In an example, a blood flow increasing mechanism can be in the first configuration when it is not in operation and can be in the second configuration when it is in operation. In an example, a blood flow increasing mechanism can transition from a first configuration to a second configuration by the extension, protrusion, twisting, and/or expansion of one or more fins, vanes, blades, or helical structures. In an example, a blood flow increasing mechanism can transition from a first configuration to a second configuration by the extension, protrusion, and/or expansion of an impeller or turbine. In an example, this extension, protrusion, twisting, and/or expansion can be caused by one or more means selected from the group consisting of: centripetal/fugal force; differential rotational an upstream member and a downstream member which connect the ends of one or more fins, vanes, blades, or helical structures; electromagnetic force; fluid resistance and/or frictional engagement; hydraulic force; inflation and/or pneumatic force; electromagnetic motors; MEMS or other microscale actuation; piezoelectric effect; or reversible shape-memory material.

In addition to the implanted blood flow lumen, this invention also includes an implanted blood flow increasing mechanism. In an example, a blood flow increasing mechanism can be an extracardiac blood pump. In an example, this blood flow increasing mechanism can increase blood flow through the implanted blood flow lumen, through a blood vessel with which the implanted blood flow lumen is in fluid communication, or both. In an example, an implanted blood flow increasing mechanism can supplement, but not replace, native blood circulation. In an example, an implanted blood flow increasing mechanism can reduce cardiac workload without completely replacing cardiac function so that the heart may still heal and recover function—avoiding the eventual need for heart transplantation or a more-invasive full-cardiac-function replacement device. In an example, a plurality of peripheral blood flow increasing mechanisms can create a system of distributed peripheral circulatory assistance.

In an example, a blood flow increasing mechanism can increase the rate, speed, volume, and/or consistency of blood flow. In an example, a blood flow increasing mechanism can also improve hemodynamics. In an example, a blood flow increasing mechanism can transduce electromagnetic energy (from a battery or other electrical power source) into kinetic energy (in the form of increased blood flow). In an example, this invention can comprise a device with a single blood flow increasing mechanism. In an example, this invention can comprise a device with multiple blood flow increasing mechanisms. In an example, multiple blood flow increasing mechanisms can be configured in parallel flow or in series flow. In an example, this invention can comprise multiple blood flow increasing mechanisms which comprise a system for distributed extracardiac circulatory assistance. In an example, a blood flow increasing mechanism can be structurally designed to avoid low-flow areas that can cause thrombogenesis. In an example, a blood flow increasing mechanism can be designed to produce hemodynamic patterns that minimize thrombogenesis.

Blood flow pumps are sometimes categorized in the field as either pulsatile or continuous. Generally, a pulsatile pump is considered to be one which produces variation in flow speed and/or pressure which is synchronized to be in phase, or out of phase, with the native cardiac pumping cycle. In an example, a blood flow increasing mechanism can be copulsating with respect to the cardiac pumping cycle. In an example, a blood flow increasing mechanism can be counterpulsating with respect to the cardiac pumping cycle. Pulsatile flow can be preferred for perfusion of some organs and can also help to reduce thrombogenesis. In an example, the blood flow increasing mechanism of this invention can produce pulsatile blood flow and/or supplement native pulsatile blood flow.

Using the terminology of the field, a blood pump can be said to produce a continuous blood flow. The designation of “continuous” can mean that a blood pump is actually intended to operate all the time, but more generally it can mean that a blood pump produces a blood flow which is not pulsatile when the pump is in operation. In other words, a continuous blood flow pump has a relatively-uniform flow speed and/or pressure as long as the pump is in operation. This distinction is important for supplemental circulation assistance devices which do not cause adverse outcomes if they are turned off (or lose power) for periods of time. Accordingly, this distinction is important for the invention disclosed herein which does not have to be in operation all the time. In an example, a continuous blood flow pump can contribute a sub-stream of continuous blood flow which is in addition to (and/or entrains) native pulsatile blood flow. In an example, the blood flow increasing mechanism of this invention can produce and contribute a continuous blood flow when it is in operation, but it does not have to be in operation all the time. In an example, the blood flow increasing mechanism of this invention can be hybrid pump which is capable of producing either a pulsatile or continuous blood flow. In an example, the operation of a blood flow increasing mechanism and the type of blood flow (e.g. pulsatile or continuous) which it produces can be controlled by a control unit for the blood flow increasing mechanism which will be discussed later in greater depth.

In an example, a blood flow increasing mechanism can be a rotary blood pump. In an example, a blood flow increasing mechanism can move blood by means of a rotating impeller or turbine. In an example, a flow increasing member can have a rotating impellor or turbine which is further comprised of one or more vanes, fins, blades, projections, winglets, airfoils, helical members, or grooves. In an example, these one or more vanes, fins, blades, projections, winglets, airfoils, or helical members can have a (first) retracted or contracted configuration in which they have a first amount of cross-sectional interaction with blood flow. In an example, these one or more vanes, fins, blades, projections, winglets, airfoils, or helical members can have a (second) protracted or expanded configuration in which they have second amount of cross-sectional interaction with blood flow. In an example, the second amount is greater than the first amount. In an example, the one or more vanes, fins, blades, projections, winglets, airfoils, helical members, or grooves transition to the second configuration when the blood flow increasing mechanism is in operation. In an example, the one or more vanes, fins, blades, airfoils, or helical members can be reversibly, repeatedly, and post-operatively moved back and forth from the first configuration to the second configuration.

In an example, this reversible, repeatable, and post-operative movement from the first configuration to the second configuration can be controlled by a control unit for the blood flow increasing mechanism. In an example, the vanes, fins, blades, airfoils, or helical members have the first configuration when the blood flow increasing mechanism is in operation and have the second configuration when the blood flow increasing mechanism is not in operation. In an example, when the blood flow increasing mechanism is in operation, it transduces electromagnetic energy into kinetic energy (in the form of blood flow). In an example, when the blood flow increasing mechanism is not in operation, it does not transduce electromagnetic energy into kinetic energy (in the form of blood flow).

In an example, a blood flow increasing member can comprise a rotating member which does not have any projecting vanes, fins, blades, projections, grooves, winglets, airfoils, and/or helical members. In an example, this blood flow increasing member can induce blood flow which is substantially perpendicular to its axis of rotation. In an example, a blood flow increasing mechanism can comprise a rotating helical or screw-shaped impeller. In an example, a blood flow increasing mechanism can comprise a rotating impeller with multiple helical or partial-helical members. In an example, a rotary pump can have one or more members which are rotated by a direct drive mechanical connection to an electromagnetic motor or other mechanical actuator. In an example, a rotary pump can have one or more magnetic members which are rotated by magnetic interaction with an electromagnetic field. In an example, a rotary blood pump can have hydrodynamic or magnetic bearings.

In an example, a blood flow increasing mechanism can be an axial rotary pump. In an example, a blood flow increasing mechanism can comprise one or more vanes, fins, blades, projections, winglets, airfoils, or helical members which rotate around an axis which is coaxial with the longitudinal axis of the blood flow lumen, with the directional vector of native blood flow, or both. In an example, a blood flow increasing mechanism can comprise one or more vanes, fins, blades, projections, winglets, airfoils, or helical members which rotate around an axis which is substantially parallel with the longitudinal axis of the blood flow lumen, with the directional vector of native blood flow, or both. In an example, a blood flow increasing mechanism can comprise one or more vanes, fins, blades, projections, winglets, airfoils, or helical members which rotate around an axis which is substantially perpendicular to the longitudinal axis of the blood flow lumen, with the directional vector of native blood flow, or both.

In an example, a blood flow increasing mechanism can move blood using peristaltic motion. In an example, a blood flow increasing mechanism can comprise a peristaltic pump. In an example, a flow increasing member can move blood by sequential compression of the lumen by a longitudinally rolling member which rolls longitudinally and compressively (from upstream to downstream) along the walls of the lumen. In an example, a flow increasing member can move blood by the sequential contraction (from upstream to downstream) of a series of circumferential members such as contracting bands or rings along the longitudinal axis of an implanted blood flow lumen. In an example, a flow increasing member can move blood by sequentially inflating and deflating a series of inflatable members such as toroidal balloons along the longitudinal axis (from upstream to downstream) of an implanted blood flow lumen. In an example, a flow increasing member can comprise a series of waving cilia-form members which wave along a lumen wall like a crowd of fans in a microscale sport arena. In an example, a flow increasing member can move blood by propagating a longitudinal wave or pulse (such as a pressure wave) longitudinally (from upstream to downstream) along a flexible membrane (or other surface) which is in fluid communication with blood in an implanted blood flow lumen.

In an example, a blood flow increasing mechanism can be selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump.

In an example, a blood flow increasing mechanism can be selected from the group consisting of: pulsatile pump; continuous pump; hybrid pulsatile and continuous pump; pump with a helical impellor; pump with an impellor with one or more airfoils; pump with an impellor with multiple vans, fins, and/or blades; pump with an impellor which rotates around an axis which is substantially parallel to the natural direction of blood flow; pump with an impellor which rotates around an axis which is substantially parallel to the longitudinal axis of the blood flow lumen; pump with an impellor which rotates around an axis which is substantially perpendicular to the natural direction of blood flow; pump with an impellor which rotates around an axis which is substantially perpendicular to the longitudinal axis of the blood flow lumen; peristaltic pump; pump with sequential circumferential contracting and/or expanding members; pump which creates longitudinal direction wave motion along a flexible surface which is in fluid communication with blood; pump with contraction and one or more one-way valves; and pump which creates blood flow by direct interaction between blood and an electromagnetic field.

In an example, a blood flow increasing mechanism can further comprise one or more moving members which increase blood flow by frictionally engaging blood and/or by entraining native blood flow. In an example, these one or more moving members can be selected from the group consisting of: airfoils, blades, fins, flippers, grooves, helical structures, rotors, threads, vanes, and winglets. In an example, the one or more moving members can have a first configuration wherein they have a first level of frictional engagement with blood flow. In an example, this first configuration can comprise being relatively close to (or flush with) a central rotating axle. In an example, this first configuration can comprise being relatively close to (or flush with) the walls of the implanted blood flow lumen. In an example, the one or more moving members can have a second configuration in which they have a second level of frictional engagement with blood flow. In an example, the second level can be substantially greater than the first level. In an example, “substantially greater” means at least 10% greater. In an example, “substantially greater” means at least 25% greater. In an example, “substantially greater” means at least 100% greater.

In an example, a blood flow increasing mechanism can further comprise one or more moving members which increase blood flow by longitudinal movement spanning a substantial portion of the cross-sectional flow area of an implanted blood flow lumen. In an example, these one or more moving members can be selected from the group consisting of: airfoils, blades, fins, flippers, grooves, helical structures, rotors, threads, vanes, and winglets. In an example, the one or more moving members can have a first configuration wherein they span a first portion of the cross-sectional flow area of an implanted blood flow lumen. In an example, this first configuration can comprise being relatively close to (or flush with) a central rotating axle. In an example, this first configuration can comprise being relatively close to (or flush with) the walls of the implanted blood flow lumen. In an example, the one or more moving members can have a second configuration in which they span a second portion of the cross-sectional flow area of an implanted blood flow lumen. In an example, the second portion can be substantially greater than the first portion. In an example, “substantially greater” means at least 10% greater. In an example, “substantially greater” means at least 25% greater. In an example, “substantially greater” means at least 100% greater.

In an example, one or more moving members of a blood flow increasing mechanism can be reversibly, repeatedly, and post-operatively transitioned from the first configuration to the second configuration by one or more means selected from the group consisting of: centripetal/fugal force, differential rotational an upstream member and a downstream member to which these members are connected, electromagnetic force, fluid resistance and/or frictional engagement, little trained gnomes, hydraulic force, inflation and/or pneumatic force, MEMS or other microscale actuation, piezoelectric effect, and reversible shape memory material. In an example, these one or more moving members can be transitioned from the first configuration to the second configuration when the blood flow increasing mechanism starts operating and can be transitioned back from the second configuration to the first configuration when the blood flow increasing mechanism stops operating.

In an example, this reversible transition allows the blood flow increasing mechanism to have a low cross-sectional profile when it is not in operation and to have a high cross-sectional profile when it is in operation. This allows the blood flow increasing mechanism to substantively supplement blood circulation when the mechanism is in operation, but to not substantively hinder native blood flow when the blood flow increasing mechanism is not in operation. In an example, the blood flow increasing mechanism can be defined to be “in operation” when it is actively transducing electromagnetic energy (such as from a battery or other electrical power source) into kinetic energy (in the form of blood flow). In an example, the ability to supplement native circulation when power is available without hindering native circulation when power is unavailable (or limited) can enable greater patient mobility and improved quality of life. This ability can also help to preserve the possibility of healing and recovery for the heart by only providing circulatory assistance when needed.

In an example, an implanted blood flow lumen and an implanted blood flow increasing mechanism can be designed so that post-implantation blood flow is greater than pre-implantation blood flow when the blood flow increasing mechanism is in operation. Further, an implanted blood flow lumen and an implanted blood flow increasing mechanism can be designed so that post-implantation blood flow is not significantly less than pre-implantation blood flow even when the blood flow increasing mechanism is not in operation.

In an example, an implanted blood flow lumen and an implanted blood flow increasing mechanism can be designed so that post-implantation cross-sectional blood flow area is greater than pre-implantation cross-sectional blood flow area (from a selected upstream location to a selected downstream location which is spanned by the implanted blood flow lumen) when the blood flow increasing mechanism is not in operation. In an example, an implanted blood flow lumen and an implanted blood flow increasing mechanism can be designed so that post-implantation resistance to blood flow (between a selected upstream location to a selected downstream location) is not substantially greater than pre-implantation resistance to blood flow between these locations when the blood flow increasing mechanism is not in operation. In an example, an implanted blood flow lumen and implanted blood flow increasing mechanism can be designed so that post-implantation blood flow capacity (between a selected upstream location to a selected downstream location) is not substantially less than pre-implantation blood flow capacity between these locations when the blood flow increasing mechanism is not in operation.

In an example, the pre-implantation minimum cross-sectional flow area can be defined as the minimum cross-sectional flow area (from a selected upstream location to a selected downstream location) before an implanted blood flow lumen and a blood flow increasing mechanism are implanted. In an example, a post-implantation minimum cross-sectional flow area can be defined as the minimum cross-sectional flow area (from the upstream location to the downstream location) which is unobstructed by the flow-increasing mechanism when the flow-increasing mechanism is not in operation, after the implanted blood flow lumen and the flow-increasing mechanism are implanted. The post-implantation minimum cross-sectional flow area can comprise the combined cross-sectional area which is available for blood flow (from the upstream location to the downstream location) through either the implanted blood flow lumen or a blood vessel. In an example, an implanted blood flow lumen and a blood flow increasing mechanism can be designed so that the post-implantation minimum cross-sectional flow area is not substantially less than the pre-implantation minimum cross-sectional flow area when a flow-increasing mechanism is not in operation. In an example, the definition of substantially less can be selected from the group consisting of: 5% less, 10% less, and 25% less.

In an example, an extracardiac circulatory assistance device can be designed to provide sufficient circulatory assistance so as to reduce cardiac workload and maintain adequate perfusion of organs, but not supplant cardiac function to such a degree that it further weakens the heart and reduces the chances of recovery without a heart transplant. In an example, the operation of a blood flow increasing mechanism can be adjusted by a control unit for the blood flow increasing mechanism to as to optimally supplement blood circulation without causing heart muscles to atrophy. In an example, a plurality of peripheral circulatory assistance devices can comprise a fluid network of “mini-hearts” which support a person's heart only to the extent which is needed during a period of cardiac healing and recovery. In an example, a plurality of extracardiac circulatory assistance devices can comprise an efficient and effective system of distributed circulatory assistance to maintain cardiac functioning and allow cardiac healing for people with CHS.

In an example, an implanted blood flow lumen can be made from one or more materials selected from the group consisting of: biological tissue (e.g. on a synthetic scaffold), cobalt chromium alloy, CoCrMo, CoCrNi, collagen, Dacron, ECM (extracellular matrix), HDPE, LDPE, material with a hydrophilic coating, nickel-titanium alloy, NiTinol, nylon, other biocompatible material, other metallic material, other polymeric material, Pebax, PET (polyethylene terephthalate), platinum, polyamide, polycaprolactone, polycarbonate, polyester, polyethylene, polyolefin, polypropylene, polytetrafluorethylene, polyurethane, PTFE (polytetrafluoroethylene), PVC (polyvinyl chloride), shape memory alloy, silocone, stainless steel, tantalum, Teflon-based materials, thermoplastic material, titanium, tungsten, urethane.

In an example, an implanted blood flow increasing mechanism can be made from one or more materials selected from the group consisting of: biological tissue (e.g. on a synthetic scaffold), cobalt chromium alloy, CoCrMo, CoCrNi, collagen, Dacron, ECM (extracellular matrix), HDPE, LDPE, material with a hydrophilic coating, nickel-titanium alloy, NiTinol, nylon, other biocompatible material, other metallic material, other polymeric material, Pebax, PET (polyethylene terephthalate), platinum, polyamide, polycaprolactone, polycarbonate, polyester, polyethylene, polyolefin, polypropylene, polytetrafluorethylene, polyurethane, PTFE (polytetrafluoroethylene), PVC (polyvinyl chloride), shape memory alloy, silocone, stainless steel, tantalum, Teflon-based materials, thermoplastic material, titanium, tungsten, urethane.

In an example, an implanted blood flow lumen and/or an implanted blood flow increasing mechanism can have an anti-thrombotic coating. In an example, an implanted blood flow lumen and/or an implanted blood flow increasing mechanism can have a coating comprising one or more substances selected from the group consisting of: anticoagulants, fibrins, heparin, heparinoids, hirudin, monoclonal antibodies, and silver.

In an example, the operation of a blood flow increasing mechanism can be controlled by a control unit for a blood flow increasing mechanism. In an example, this control unit can be located locally in direct mechanical communication with the blood flow increasing mechanism. In an example, such a local control unit can further comprise an actuation mechanism (such as a motor) which moves or otherwise actuates the blood flow increasing mechanism. In an example, a local control unit can further comprise one or more members selected from the group consisting of: motor, power source, power transducer, data processor, digital member, and wireless communication module. In an example, a control unit can be in a remote location and in wireless communication with the blood flow increasing mechanism.

In an example, the control unit for a blood flow increasing mechanism can activate or deactivate the blood flow increasing mechanism. In an example, a control unit can change the blood flow rate produced by a blood flow increasing mechanism. In an example, a control unit can change a produced blood flow mode from a pulsatile flow to a continuous flow. In an example, a control unit can change the torque of a rotating impeller on a blood flow increasing mechanism. In an example, a control unit can activate one or more moving members of a blood flow increasing mechanism to reversibly, repeatedly, and post-operatively transition from a first configuration (with less obstruction of lumen cross-sectional blood flow area) to a second configuration (with more obstruction of lumen cross-sectional blood flow area) when the blood flow increasing mechanism is in operation.

In an example, a control unit for a blood flow increasing member can be programmable. In an example, a control unit for a blood flow increasing member can be in wireless communication with a remote computer, human-to-computer interface, and/or computer-to-human interface which allows the control unit to be reprogrammed (or otherwise adjusted) in a non-invasive and ongoing manner (long after implantation). In an example, a control unit for a blood flow increasing member can be remotely reprogrammed (or otherwise adjusted) by a healthcare professional. In an example, a control unit for a blood flow increasing member can autonomously change the operation of a blood flow increasing mechanism in response to data from one or more implanted sensors. In an example, a control unit for a blood flow increasing member can autonomously change the operation of a blood flow increasing mechanism in response to data from one or more wearable sensors.

In an example, the control unit for a blood flow increasing member can adjust the operation of a blood flow increasing mechanism based on data received from an implanted or wearable ECG monitor or from another type of cardiac function sensor. In an example, the control unit for a blood flow increasing member can adjust the operation of a blood flow increasing mechanism based on data received from one or more sensors which measure the oxygenation levels of body fluid, tissue, and/or organs. In an example, the control unit for a blood flow increasing member can adjust the operation of a blood flow increasing mechanism based data from one or more sensors which measure hemodynamic parameters. In an example, a control unit for a blood flow increasing member can adjust the operation of a blood flow increasing mechanism based on data from one or more sensors which measure blood flow rates, blood pressure levels, and/or blood pressure differentials.

In an example, the control unit for a blood flow increasing member can adjust the operation of a blood flow increasing mechanism based on changes in blood viscosity or the detection of thrombogenic conditions by one or more implanted sensors. In an example, the control unit for a blood flow increasing member can adjust the operation of a blood flow increasing mechanism based on the stored amount electrical power in a battery, the ability of alternative energy sources which can be transduced into electrical power, and/or the availability of external electrical power. In an example, a control unit for a blood flow increasing member can adjust the operation of a blood flow increasing mechanism based on secure input and/or commands which are remotely (wirelessly) received from a health care provider.

In an example, the control unit for a blood flow increasing mechanism can change the operation of the blood flow increasing mechanism based on one or more physiological or environmental factors selected from the group consisting of: bioimpedance, blood oxygen saturation, blood pressure or pressure differentials, blood viscosity level, blood cell count, body movement, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, a control unit for a blood flow increasing mechanism can change the operation of the blood flow increasing mechanism based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this invention can further comprise one or more additional components selected from the group consisting of: a power source, a power transducer and/or energy harvester, an electric motor, a data processing unit, a digital memory, a wireless data receiver and/or transmitter, a (one-way) fluid valve, an implanted sensor, and a (reversibly and automatically deployable) thrombus-catching net, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In an example, a power source, power transducer, and/or energy harvester can supply and/or transduce electromagnetic power from one or more sources selected from the group consisting of: a rechargeable or replaceable battery, an energy-storing electronic chip, energy transmitted through inductively-coupled coils, energy harvested and/or transduced from body thermal energy (such as using Peltier effects), energy harvested and/or transduced from body motion or kinetic energy (such as muscle motion), energy harvested and/or transduced via piezoelectric members, energy harvested and/or transduced from ambient and/or external electromagnetic energy, energy from an external power source, energy harvested and/or transduced from biochemical and/or biological processes, and energy harvested and/or transduced from light energy.

In an example, a data processing unit can perform one or more functions selected from the group consisting of: control motor function, receive and analyze sensor data, run software programs, and store data in memory. In an example, a wireless data receiver and/or transmitter can perform one or more functions selected from the group consisting of: transmit and receive data via Bluetooth, WiFi, Zigbee, or other wireless communication modality; transmit and receive data to and from a mobile electronic device such as a cellular phone, mobile phone, smart phone, electronic tablet; transmit and receive data to and from a wearable device such as a smart watch or electronically-functional eyewear; transmit and receive data to and from the internet; send and receive electronic messages; and transmit and receive data to and from a different implantable medical device.

In an example, a fluid valve can be a one-way valve. In an example, a fluid valve can have multiple leaflets. In an example, a fluid valve can be bicuspid (with two leaflets). In an example, a fluid valve can have three leaflets. In an example, a fluid valve can be a ball check valve. In an example, a fluid valve can comprise a flap over an opening.

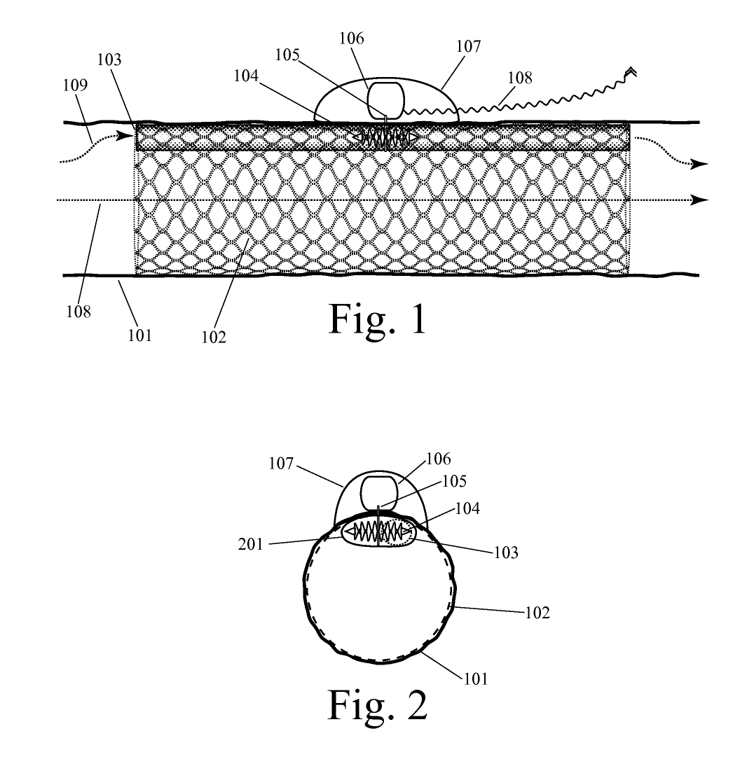

Having provided an introduction to the figures, we now discuss FIGS. 1 through 98 in detail.

FIGS. 1 through 98 show examples of how this invention can be embodied in an implanted extracardiac device for supplementing blood circulation. However, these figures do not limit the full generalizability of the claims. Also, the variations in design and components which were just discussed in the preceding portions of this section can be variously applied to the examples shown in these figures in order to create variations and additional examples which are within the scope of this invention and its claims, even if these variations are not repeated in discussions which accompany each of the individual figures.

FIGS. 1 and 2 show two perspectives of an example of how this invention can be embodied in an implanted device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

In the example shown in FIGS. 1 and 2, the implanted blood flow lumen is a stent with two blood flow channels. In this example, the blood flow increasing mechanism is a rotary blood pump. In this example, the stent and blood pump are both configured to be implanted substantially within the walls of a blood vessel. FIGS. 1 and 2 show two different cross-sectional views of this example. FIG. 1 shows a longitudinal semi-transparent view of this device. FIG. 2 shows a lateral cross-sectional semi-transparent view of this same device.

FIG. 1 shows a longitudinal view of the walls of blood vessel 101 in order to show the anatomical context in which this device is used. FIG. 1 also shows stent 102 after it has been inserted and expanded within blood vessel 101. Methods for inserting and expanding stents in blood vessels are well known in the prior art (including insertion by a catheter and expansion by an inflatable member) and the specifics of stent insertion and expansion are not central to this example. FIG. 1 shows this device after insertion and expansion have occurred. In this example, stent 102 comprises a generally-cylindrical radially-expandable metal net or mesh. In other examples, stent 102 can be comprised of a polymer or biological material. In other examples, stent 102 can have multiple layers or can have a non-circular cross-section. In this example, the post-expansion interior of stent 102 includes a first blood flow channel through which blood can flow in an unobstructed manner. In this example, there is no mechanism in this first blood flow channel for accelerating blood flow and also no mechanism that might hinder blood flow.

FIG. 1 also shows a longitudinal semi-transparent view of a second blood flow channel 103. In this example, second blood flow channel 103 is a generally-cylindrical tube that is inside stent 102 and connected to the wall of stent 102. In this example, the longitudinal axis of second blood flow channel 103 is generally parallel to the longitudinal axis of stent 102. In this example, second blood flow channel 103 spans substantially the entire length of stent 102. In an example, a second blood flow channel can span only a portion of the length of a stent. In an example, a second blood flow channel can protrude outwards from the ends of a stent.

FIG. 1 also shows a blood flow increasing mechanism that accelerates the flow of blood through second blood flow channel 103. In this example, the blood flow increasing mechanism in a rotary blood pump that further comprises: rotating turbine, impeller, or blade 104; rotating axle 105; motor or actuator 106; housing 107; and electrical power wire 108. In this example, electrical power from a power source delivered through electrical power wire 108 powers motor or actuator 106, which rotates axle 105, which rotates turbine, impeller, or blade 104, which accelerates blood flow through second blood flow channel 103. In an example, motor or actuator 106, housing 107, and electrical power wire could alternatively be viewed as comprising a control unit for the blood flow increasing mechanism.

In this example, blood flow through blood vessel 101 diverges at an upstream location and separates into a first blood flow stream that flows through the first blood flow channel and a second blood flow stream that flows through the second blood flow channel. Blood flow through the second blood flow stream is accelerated by the blood flow increasing mechanism. Then, blood flow from the first blood flow channel and blood flow from the second blood flow channel reconverge at a downstream location. In an example, accelerated blood flow from the second blood flow channel can accelerate blood flow from first blood flow channel by entrainment. When blood flows from the first and second channels converge at the downstream location, the total blood flow from the upstream location to the downstream location is accelerated.

As shown in FIG. 1, a blood flow increasing mechanism can be a blood pump with a rotating turbine, impeller, rotor, and/or blade that is located at least partially within a second blood flow channel, wherein this rotating turbine, impeller, rotor, and/or blade can be rotated by the rotation of an axle, and wherein this axle is mechanically connected to a motor and/or actuator. In an example, a blood flow increasing mechanism can include: a rotating turbine, impeller, rotor, and/or blade that is configured to be located within a blood vessel; and a motor or actuator that is configured to be located outside the blood vessel, wherein the turbine, impeller, rotor, and/or blade is rotated by a leak-proof mechanical connection through the blood vessel wall to the motor or actuator.

As shown in FIG. 1, a blood flow increasing mechanism can comprise a pump with a rotating turbine, impeller, or blade that is located at least partially located within the second blood flow channel, wherein this rotating turbine, impeller, or blade can be rotated around an axis that is substantially perpendicular to one or more vectors selected from the group consisting of: the vector comprising the longitudinal axis of the blood vessel; the vector comprising the longitudinal axis of the second blood flow channel; the vector comprising the direction of blood flow through the blood vessel; and vector comprising the direction of blood flow through the second blood flow channel.

In an example, a blood flow increasing mechanism can be a pump with a rotating turbine, impeller, rotor, and/or blade. In other examples, a blood flow increasing mechanism can comprise another type and configuration of pump. In an example, a pump can be selected from the group consisting of: biochemical pump, elastomeric pump, electromagnetic pump, electromechanical pump, Micro Electro Mechanical System (MEMS) pump, osmotic pump, peristaltic pump, piezoelectric pump, pump with an expansion chamber and one-way valve, rotating blade pump, rotating impeller pump, and rotating turbine pump.

In an example, a blood flow increasing mechanism can be powered by an implanted battery, energy-storing chip, or capacitor. In an example, an implanted battery, energy-storing chip, or capacitor can be recharged from an external source by electromagnetic inductance. In an example, a blood flow increasing mechanism can be directly powered from an external energy source.

In various embodiments of this invention, a blood flow increasing mechanism can be powered from one or more energy sources selected from the group consisting of: energy from an internal battery, energy-storing chip, or capacitor; energy from external source via electromagnetic inductance; energy harvested or transduced from a bioelectrical cell; energy harvested or transduced from an electromagnetic field; energy harvested or transduced from blood flow or other internal fluid flow; energy harvested or transduced from body kinetic energy; energy harvested or transduced from ions or glucose in saliva or elsewhere in the body; energy harvested or transduced from kinetic, mechanical, thermal, chemical, or biological energy from a person's body; energy harvested or transduced from muscle activity; energy harvested or transduced from organ motion; and energy harvested or transduced from thermal energy.

FIG. 2 shows a lateral cross-sectional semi-transparent view of the same device that was shown in FIG. 1. FIG. 2 shows a lateral cross-sectional view of the generally-circular cross-sectional wall of blood vessel 101. FIG. 2 also shows a lateral cross-sectional view of the generally-circular cross-sectional wall of stent 102. FIG. 2 also shows a lateral cross-sectional view of the generally-circular cross-sectional wall of second blood flow channel 103.

FIG. 2 also shows lateral cross-sectional views of the components of the blood accelerating mechanism including: rotating turbine, impeller, or blade 104; rotating axle 105; motor or actuator 106; and housing 107. The view of electrical power wire 108 is obscured from this perspective. Visible for the first time in the perspective in FIG. 2 is a central bulge 201 in the second blood flow channel that encircles rotating turbine, impeller, or blade 104 so that rotation of turbine, impeller, or blade 104 accelerates blood flow through second blood flow channel 103.

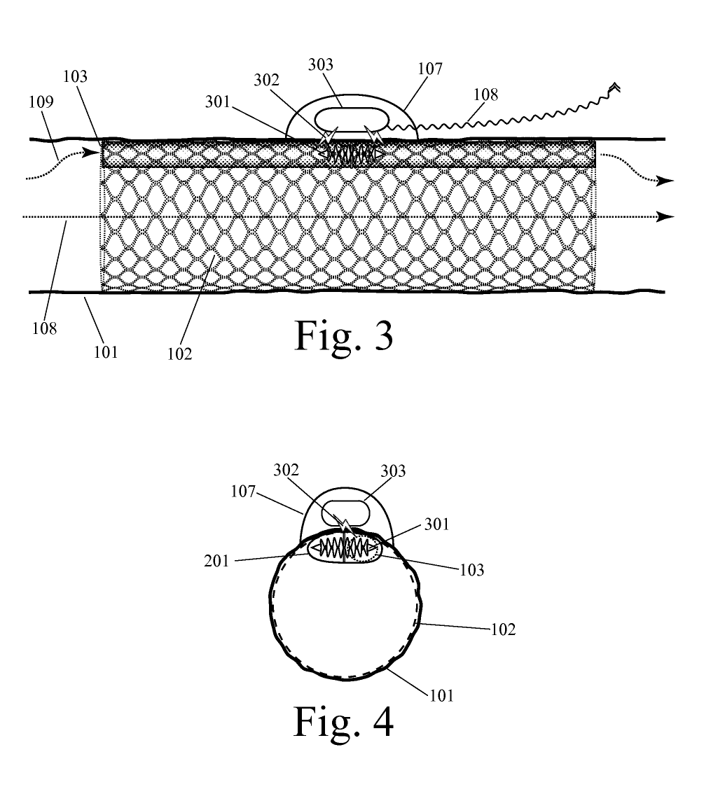

FIGS. 3 and 4 show an example of how this invention can be embodied that is similar to that shown in FIGS. 1 and 2 except that the turbine, impeller, or blade is rotated by magnetic interaction with an electromagnetic field instead of by a direct mechanical connection with a motor or actuator through a rotating axle. This design avoids the challenges of creating a leak-proof seal for the rotating axle where it goes through the wall of the blood vessel. FIG. 3 shows a longitudinal semi-transparent view of the device. FIG. 4 shows a lateral cross-sectional semi-transparent view of this same device.

Device components in FIGS. 3 and 4 that are different than those in FIGS. 1 and 2 include: electromagnetically-interactive turbine, impeller, or blade 301; electromagnetic field 302 (represented symbolically by lightning bolt symbols); and electromagnetic energy emitting member 303. Various methods for causing a turbine, impeller, or blade to rotate by interaction with an electromagnetic field (including field oscillations and parallel magnet rotation) are known in the prior art and the precise method is not central to this invention.

As shown in the example in FIGS. 3 and 4, a blood flow increasing mechanism can include: a rotating turbine, impeller, or blade that is configured to be located within a blood vessel; and an electromagnetic energy emitting member that is configured to be located outside the blood vessel, wherein the turbine, impeller, or blade is rotated by interaction with an electromagnetic field created by the electromagnetic energy emitting member without requiring a direct mechanical connection between the member and the turbine, impeller, or blade. In an example, a blood flow increasing mechanism can be a pump with a rotating turbine, impeller, or blade that is located at least partially within the second blood flow channel and wherein this turbine, impeller, or blade is rotated by interaction with an electromagnetic field without requiring a mechanical connection to a motor and/or actuator.

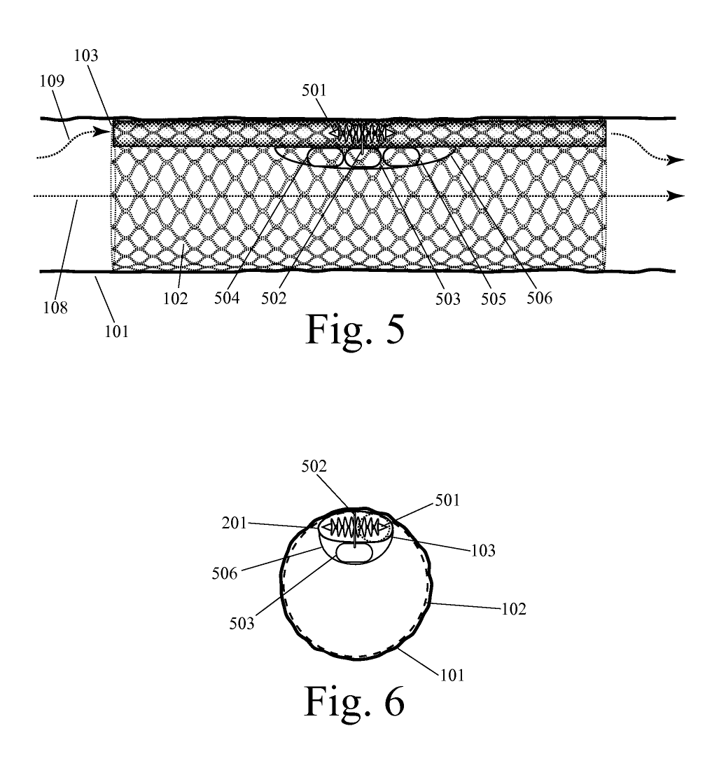

FIGS. 5 and 6 show an example of how this invention can be embodied that is similar to that shown in FIGS. 1 and 2, except that all components are now located completely within the blood vessel. The primary challenges of this design include: minimizing the intrusion of the cross-sectional profile of the blood flow increasing mechanism into the cross-sectional area of the blood vessel that is available for native flow; and the power source for the blood flow increasing mechanism that is now located entirely within the blood vessel. The primary advantage of this design is that it can be implanted entirely in an endovascular and minimally-invasive manner.

FIG. 5 shows a longitudinal semi-transparent view and FIG. 6 shows a lateral cross-sectional semi-transparent view. Device components in FIGS. 5 and 6 that are different than those in FIGS. 1 and 2 include: rotating turbine, impeller, or blade 501; rotating axle 502; intra-vessel motor or actuator 503; power source 504; data processing and wireless communication unit 505; and housing 506.

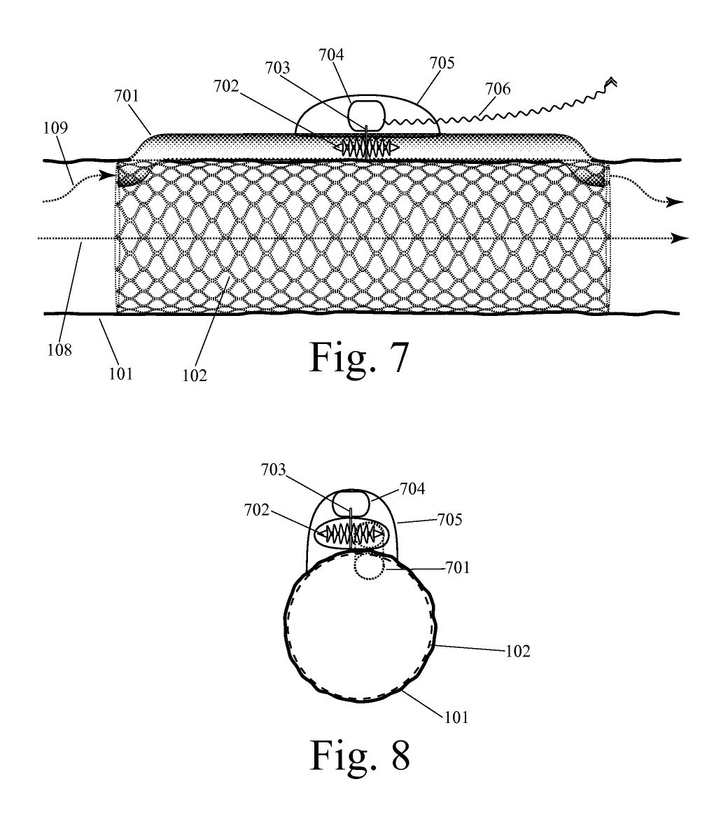

FIGS. 7 and 8 show an example of how this invention can be embodied that is similar to that shown in FIGS. 1 and 2 except that a portion of the second blood flow channel and the rotating turbine, impeller, or blade are located outside of the blood vessel. In an example, a second blood flow channel is located at least partially outside the blood vessel. The primary challenges of this design include: having to attach the second blood flow channel to the outside of the blood vessel; and inserting and connecting the ends of the second blood flow channel to the stent through the blood vessel walls with minimal tissue damage and blood hemorrhaging. The primary advantage of this design is that it enables low-profile intrusion into the cross-sectional area of the blood vessel that is available for native blood flow.

FIG. 7 shows a longitudinal semi-transparent view and FIG. 8 shows a lateral cross-sectional semi-transparent view. Device components in FIGS. 7 and 8 that are different than those in FIGS. 1 and 2 include: outside vessel second blood flow channel 701; outside vessel rotating turbine, impeller, or blade 702; outside vessel rotating axle 703; motor or actuator 704, housing 705, and electrical power wire 706.

In an example, this invention can be embodied in a device that includes connection ports on the stent for externally attaching one or both ends of a second blood flow channel to a stent through a blood vessel wall with minimal blood loss and/or tissue trauma. In an example, connection ports can include one or more members selected from the group consisting of: spiral threads; circular ridges, beveled ridges, fluid seal, gel seal, adhesive seal, interlocking tongue and groove, twist connection, snapping member, automatic-cauterizing member, with drawstring, pull-tie, and interlocking joints.

In the example shown in FIG. 7, the upstream end of second blood flow channel 701 is configured to extend into the interior of blood vessel 101 with an upstream-facing funnel shape to intake blood along a vector that is generally parallel to the longitudinal axis of the blood vessel. In this example, the downstream end of second blood flow channel 701 is configured to extend into the interior of blood vessel 101 with a downstream-facing funnel shape to eject blood along a vector that is generally parallel to the longitudinal axis of the blood vessel. In an example, blood flow exiting the second blood flow channel can help to accelerate blood flow through the blood vessel via entrainment.

In an alternative example, the ends of second blood flow channel 701 can be configured to be substantially flush with the blood vessel walls and/or the walls of the stent, as with a surgical anastomosis. In this case, the ends of the second blood flow channel 701 would not extend substantially into the interior of blood vessel 101. In this case, the ends of the second blood flow channel 701 would intake and eject blood along vectors that are generally perpendicular to the longitudinal axis of the blood vessel.

An alternative design with ends that are flush with the vessel and/or stent walls has the advantage of minimal, if any, intrusion into the cross-sectional area of the blood vessel. This minimizes resistance to unaided blood flow through the vessel. However, this alternative design may be less efficient for entraining and accelerating blood flow through the blood vessel because blood is not ejected from the second blood flow channel along a vector that is parallel to the longitudinal flow of blood through the blood vessel. In an example, a one-way flow valve could be added to the stent to encourage forward flow. In an example, such a one-way flow valve could be added between the upstream end and the downstream end of the second blood flow channel to encourage forward flow. In an example, this one-way flow valve can be similar to those used within the heart.

In an example, a blood flow increasing mechanism can include: a rotating turbine, impeller, or blade that is configured to be located within the blood vessel; and an electromagnetic energy emitting member that is configured to be located inside the blood vessel, wherein the turbine, impeller, or blade is rotated by interaction with an electromagnetic field created by the electromagnetic energy emitting member without a direct mechanical connection between the member and the turbine, impeller, or blade.

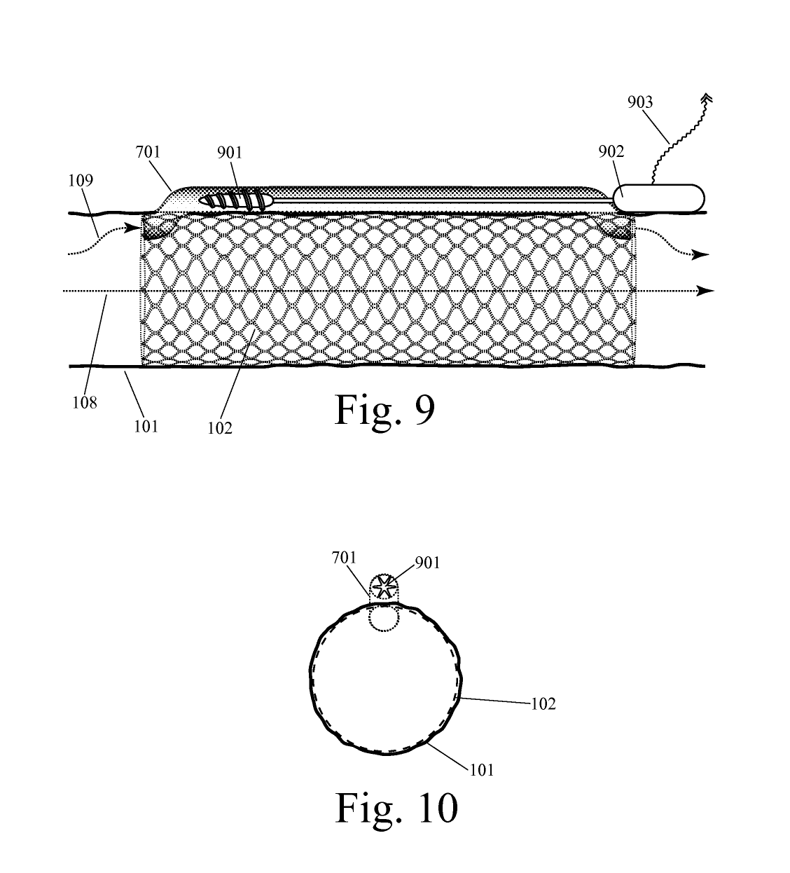

FIGS. 9 and 10 show an example of how this invention can be embodied that is similar to that shown in FIGS. 7 and 8 except that the rotating turbine, impeller, or blade rotates around an axis that is generally parallel to the longitudinal axis of the second blood flow lumen. This design can decrease the profile of the device protruding out from the outer wall of the blood vessel. FIG. 9 shows a longitudinal view and FIG. 10 shows a lateral cross-sectional view. Device components in FIGS. 9 and 10 that are different than those in FIGS. 7 and 8 include: longitudinal-axle rotating turbine, impeller, or blade 901; motor or actuator 902, and electrical power wire 903.

In an example, a blood flow increasing mechanism can comprise a pump with a rotating turbine, impeller, or blade that is located at least partially within a second blood flow channel that rotates around an axis that is substantially parallel to one or more vectors selected from the group consisting of: the vector comprising the longitudinal axis of the blood vessel; the vector comprising the longitudinal axis of the second blood flow lumen; the vector comprising the direction of blood flow through the blood vessel; and vector comprising the direction of blood flow through the second blood flow lumen.

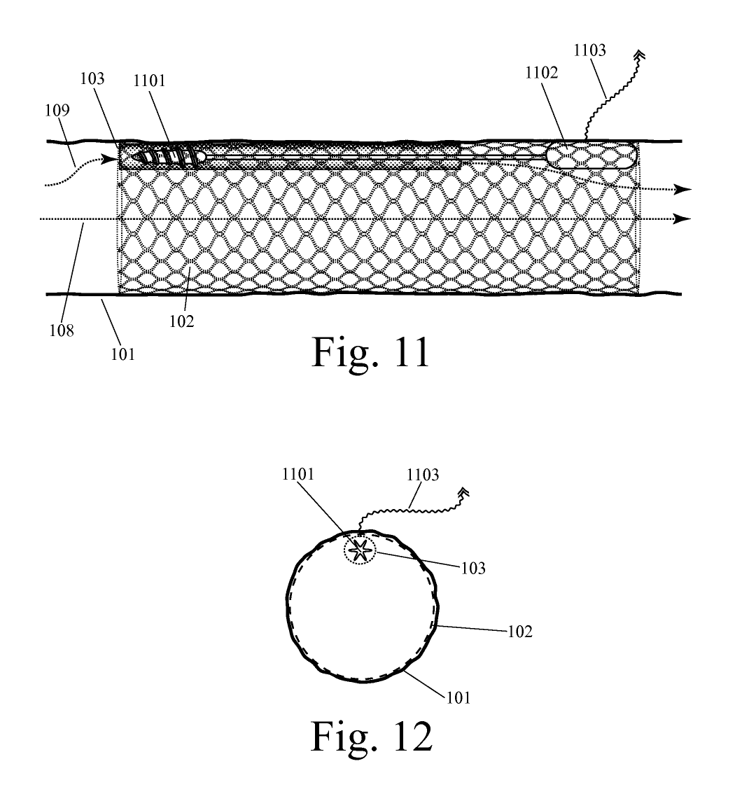

FIGS. 11 and 12 show an example of how this invention can be embodied that is similar to that shown in FIGS. 5 and 6 except that the rotating turbine, impeller, or blade rotates around an axis that is generally parallel to the longitudinal axis of the second blood flow lumen. This design can decrease the profile of the device protruding into the cross-sectional area of the blood vessel. Device components in FIGS. 11 and 12 that are different than those in previous figures include: longitudinal-axle rotating turbine, impeller, or blade 1101; motor or actuator 1102, and electrical power wire 1103.

In an example, a blood flow increasing mechanism can be powered by a power source that is configured to be external to a blood vessel. In an example, a blood flow increasing mechanism can be powered by a power source that is configured to be inside a blood vessel. In an example, a blood flow increasing mechanism can be powered by a battery, energy-storing chip, or capacitor. In an example, a battery, energy-storing chip, or capacitor can be recharged from an external source by electromagnetic inductance.

In various embodiments of this invention, a blood flow increasing mechanism can be powered from one or more energy sources selected from the group consisting of: energy from an internal battery, energy-storing chip, or capacitor; energy from external source via electromagnetic inductance; energy harvested or transduced from a bioelectrical cell; energy harvested or transduced from an electromagnetic field; energy harvested or transduced from blood flow or other internal fluid flow; energy harvested or transduced from body kinetic energy; energy harvested or transduced from ions or glucose in saliva or elsewhere in the body; energy harvested or transduced from kinetic, mechanical, thermal, chemical, or biological energy from a person's body; energy harvested or transduced from muscle activity; energy harvested or transduced from organ motion; and energy harvested or transduced from thermal energy.

In an example, a blood flow increasing mechanism can include: a rotating turbine, impeller, or blade that is configured to be located within a blood vessel; and a motor or actuator that is configured to be located within the blood vessel, wherein the turbine, impeller, or blade is rotated by mechanical connection to the motor or actuator. In an example, a second blood flow channel can be located entirely within a blood vessel.

In an example, a device can be configured to be implanted inside a blood vessel so that the entire device can be implanted in an endovascular manner. In an example, a stent and a first blood flow channel can be configured to be implanted inside the blood vessel so that they can be implanted in an endovascular manner and a blood flow increasing mechanism and the second blood flow channel can be externally attached to the outside of the blood vessel.

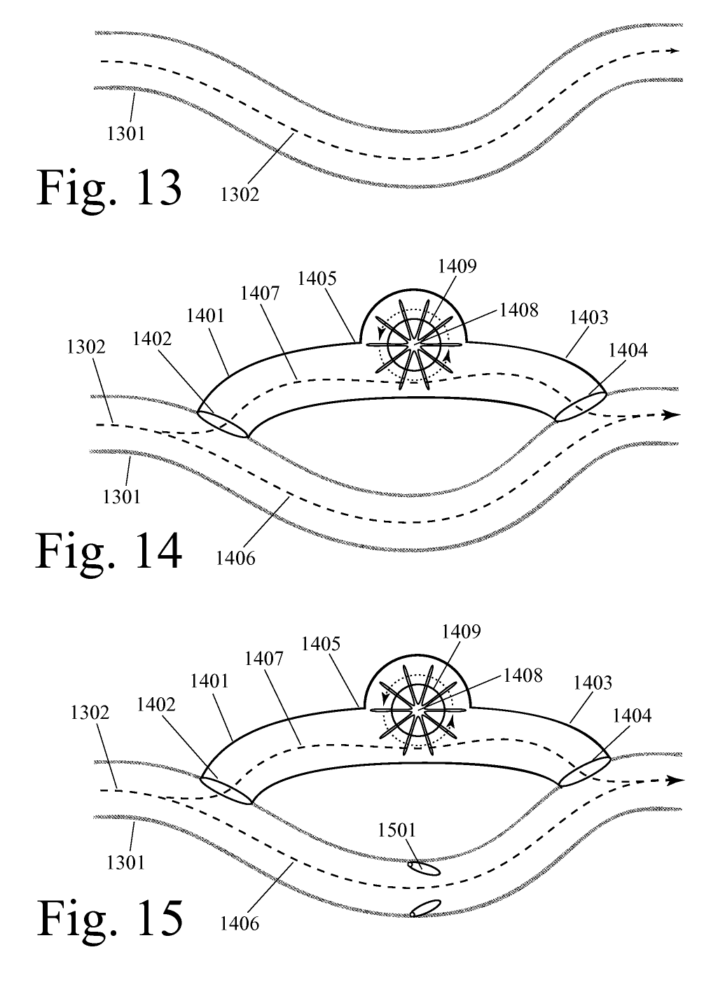

FIGS. 13 and 14 show an example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 13 shows a view of the blood vessel before the device is implanted. FIG. 13 is shown to provide the anatomical context for device implantation. FIG. 13 shows blood vessel 1301 and blood flow 1302 through this blood vessel. FIG. 14 shows a view of this blood vessel after a device has been implanted. In addition to blood vessel 1301 and blood flow 1302, FIG. 14 also shows an implanted blood flow lumen (further comprising an upstream lumen portion 1401, a middle lumen portion 1405, and a downstream lumen portion 1403) which is connected to blood vessel 1301 by upstream anastomosis 1402 and by downstream anastomosis 1404. In this example, the implanted blood flow lumen is an artificial vessel segment. FIG. 14 also shows an implanted blood flow increasing mechanism comprising rotating impeller 1408 as well as a control unit 1409 for the blood flow increasing mechanism. In this example, control unit 1409 can further comprise a motor and power source. In this example, rotating impeller 1408 rotates around an axis which is substantially perpendicular to the longitudinal axis of the implanted blood flow lumen and/or the directional vector of blood flow through the implanted blood flow lumen.

In the example shown in FIG. 14, the implanted blood flow lumen causes a bifurcation of blood flow 1302. In this example, the portion of the blood flow which splits off into the implanted blood flow lumen is blood flow 1407 and the portion of the blood flow which continues through the blood vessel is blood flow 1406. In this example, the rotation of impeller 1408 increases blood flow 1407, which increases the combined blood flow 1406 and 1407 through the implanted blood flow lumen and the original blood vessel from an upstream location (anastomosis 1402) to a downstream location (anastomosis 1404). In an example, blood flow 1407 accelerates blood flow 1406 via entrainment when they reconverge at the downstream location.

In the example shown in FIG. 14, the post-implantation cross-sectional flow area available for blood to flow from an upstream location (anastomosis 1402) to a downstream location (anastomosis 1404) is not substantially less than the pre-implantation cross-sectional flow area available for blood flow between these locations in the original blood vessel alone, regardless of whether the blood flow increasing mechanism (impeller 1408) is operating or not. In this manner, this device does not hinder or restrict native blood flow when the blood flow increasing mechanism is not operating.

FIGS. 13 and 14 show an example of a device wherein: (a) a pre-implantation minimum cross-sectional flow area is the minimum cross-sectional flow area (from the upstream location to the downstream location) before the implanted blood flow lumen and the blood flow increasing mechanism are implanted; (b) a post-implantation minimum cross-sectional flow area is the minimum cross-sectional flow area (from the upstream location to the downstream location) which is unobstructed by the blood flow increasing mechanism when the blood flow increasing mechanism is not in operation after the implanted blood flow lumen and the blood flow increasing mechanism are implanted; and (c) the post-implantation minimum cross-sectional flow area is not substantially less than the pre-implantation minimum cross-sectional flow area. In an example, the definition of substantially less can be selected from the group consisting of: 5% less, 10% less, and 25% less.

FIGS. 13 and 14 show an example of a device wherein post-implantation blood flow (from the upstream location to the downstream location) is greater than pre-implantation blood flow (from the upstream location to the downstream location) when the blood flow increasing mechanism is in operation transducing electromagnetic energy into kinetic energy. FIGS. 13 and 14 also show an example of a device wherein post-implantation blood flow (from the upstream location to the downstream location) when the blood flow increasing mechanism is not in operation is not substantially less than pre-implantation blood flow (from the upstream location to the downstream location). In an example, the definition of substantially less can be selected from the group consisting of: 5% less, 10% less, and 25% less.

FIGS. 13 and 14 show an example of a device wherein an implanted blood flow lumen is configured to be implanted at least partially outside a blood vessel. In an example, the post-implantation minimum cross-sectional flow area can comprise the combined cross-sectional area through which blood flows unobstructed (from the upstream location to the downstream location) through either the implanted blood flow lumen or the blood vessel with which it is in fluid communication.

FIGS. 13 and 14 show an example of a device wherein an implanted blood flow lumen is configured to be implanted into fluid communication with a blood vessel by one or more connecting members or connection methods which are selected from the group consisting of: endovascular insertion and expansion within a blood vessel, anastomosis, sutures, purse string suture, drawstring, pull tie, friction fit, surgical staples, tissue adhesive, gel, fluid seal, biochemical bond, cauterization, (three-way) vessel joint, vessel branch, twist connector, helical threads or screw connector, connection port, interlocking joints, tongue and groove connection, flanged connector, beveled ridge, magnetic connection, plug connector, circumferential ring, inflatable ring, and snap connector. In particular, FIG. 14 shows an example of a device wherein an implanted blood flow lumen is configured to be implanted into fluid communication with a blood vessel by one or more surgical anastomoses.

FIG. 14 shows an example of a device wherein an implanted blood flow lumen is selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber. In particular, FIG. 14 shows an example of a device wherein an implanted blood flow lumen is an artificial vessel segment.

FIG. 14 shows an example of a device wherein a blood flow increasing mechanism is selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump. In particular, FIG. 14 shows an example of a device wherein a blood flow increasing mechanism is an axial rotary pump.

FIG. 15 shows an example of a device that is like the one shown in FIG. 14 except that it further includes a one-way flow valve 1501. In this example, there is one such valve and it is configured to be inserted within the portion of the natural blood vessel between the upstream location and the downstream location.

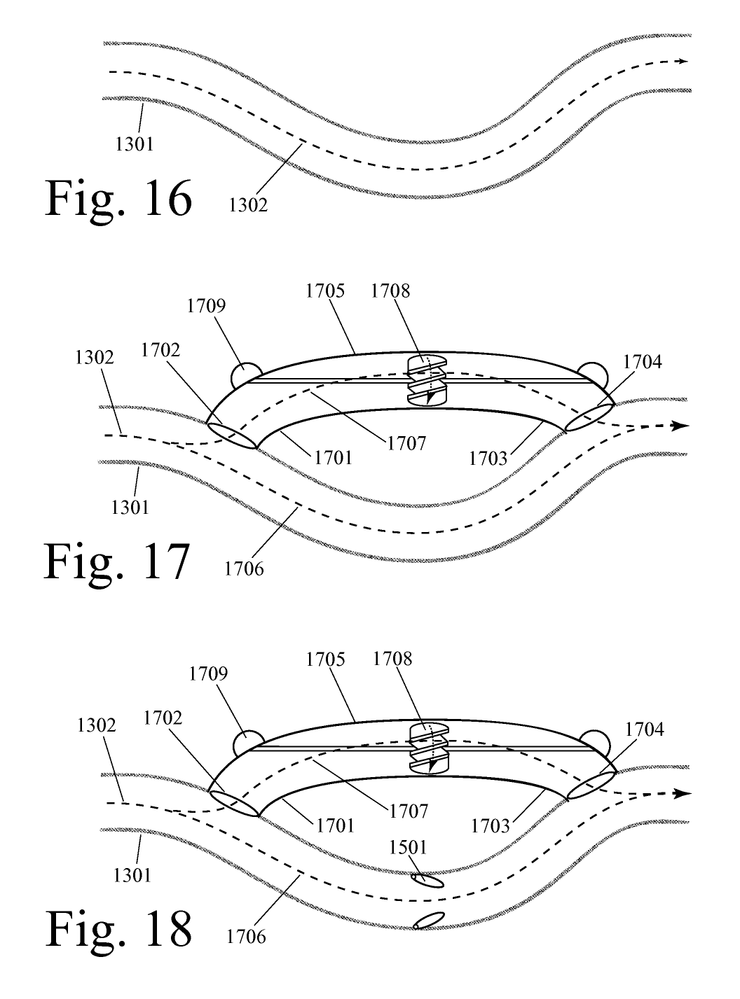

FIGS. 16 through 18 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

The example shown in FIGS. 16 through 18 is similar to the example shown in FIGS. 13 through 15, except that the blood flow increasing mechanism now comprises an axial rotary pump with an impeller which rotates around an axis which is substantially parallel to: the longitudinal axis of the implanted blood flow lumen; and/or the directional vector of blood flow through the implanted blood flow lumen.

FIG. 16 shows the blood vessel before the device is implanted in order to show anatomical context for device implantation. FIG. 16 shows blood vessel 1301 and blood flow 1302 through this blood vessel. FIG. 17 shows this blood vessel after the device has been implanted. In addition to blood vessel 1301 and blood flow 1302, FIG. 17 also shows an implanted blood flow lumen (further comprising an upstream lumen portion 1701, a middle lumen portion 1705, and a downstream lumen portion 1703) which is connected to blood vessel 1301 by upstream anastomosis 1702 and by downstream anastomosis 1704. In this example, the implanted blood flow lumen is an artificial vessel segment. FIG. 17 also shows an implanted blood flow increasing mechanism comprising rotating impeller 1708 as well as a control unit 1709 for the blood flow increasing mechanism. In this example, control unit 1709 can further comprise a motor and power source. In this example, rotating impeller 1708 rotates around an axis which is substantially parallel to the longitudinal axis of the implanted blood flow lumen and/or the directional vector of blood flow through the implanted blood flow lumen.

In the example shown in FIG. 17, the implanted blood flow lumen causes a bifurcation of blood flow 1302. In this example, the portion of the blood flow which splits off into the implanted blood flow lumen is blood flow 1707 and the portion of the blood flow which continues through the blood vessel is blood flow 1706. In this example, the rotation of impeller 1708 increases blood flow 1707, which increases the combined blood flow 1706 and 1707 through the implanted blood flow lumen and the original blood vessel from an upstream location (anastomosis 1702) to a downstream location (anastomosis 1704). In an example, blood flow 1707 accelerates blood flow 1706 via entrainment when they reconverge at the downstream location.

In the example shown in FIG. 17, the post-implantation cross-sectional flow area available for blood flow from an upstream location (anastomosis 1702) to a downstream location (anastomosis 1704) is not substantially less than the pre-implantation cross-sectional flow area available for blood flow between these locations in the original blood vessel alone, regardless of whether the blood flow increasing mechanism (impeller 1708) is operating or not. In this manner, this device does not hinder or restrict native blood flow when the blood flow increasing mechanism is not operating.

FIGS. 16 and 17 show an example of a device wherein: (a) a pre-implantation minimum cross-sectional flow area is the minimum cross-sectional flow area (from the upstream location to the downstream location) before the implanted blood flow lumen and the blood flow increasing mechanism are implanted; (b) a post-implantation minimum cross-sectional flow area is the minimum cross-sectional flow area (from the upstream location to the downstream location) which is unobstructed by the blood flow increasing mechanism when the blood flow increasing mechanism is not in operation after the implanted blood flow lumen and the blood flow increasing mechanism are implanted; and (c) the post-implantation minimum cross-sectional flow area is not substantially less than the pre-implantation minimum cross-sectional flow area. In an example, the definition of substantially less can be selected from the group consisting of: 5% less, 10% less, and 25% less.

FIGS. 16 and 17 show an example of a device wherein post-implantation blood flow (from the upstream location to the downstream location) is greater than pre-implantation blood flow (from the upstream location to the downstream location) when the blood flow increasing mechanism is in operation transducing electromagnetic energy into kinetic energy. FIGS. 16 and 17 also show an example of a device wherein post-implantation blood flow (from the upstream location to the downstream location) when the blood flow increasing mechanism is not in operation is not substantially less than pre-implantation blood flow (from the upstream location to the downstream location). In an example, the definition of substantially less can be selected from the group consisting of: 5% less, 10% less, and 25% less.

FIGS. 16 and 17 show an example of a device wherein an implanted blood flow lumen is configured to be implanted at least partially outside a blood vessel. In an example, the post-implantation minimum cross-sectional flow area can comprise the combined cross-sectional area through which blood flows unobstructed (from the upstream location to the downstream location) through either the implanted blood flow lumen or the blood vessel with which it is in fluid communication. FIG. 17 shows an example of a device wherein an implanted blood flow lumen is configured to be implanted into fluid communication with a blood vessel by one or more surgical anastomoses. FIG. 17 shows an example of a device wherein an implanted blood flow lumen is an artificial vessel segment. FIG. 17 shows an example of a device wherein a blood flow increasing mechanism is an axial rotary pump. FIG. 18 shows an example of a device that is like the one shown in FIG. 17 except that it further includes a one-way flow valve 1501. In this example, there is one such valve and it is configured to be inserted within the portion of the natural blood vessel between the upstream location and the downstream location.

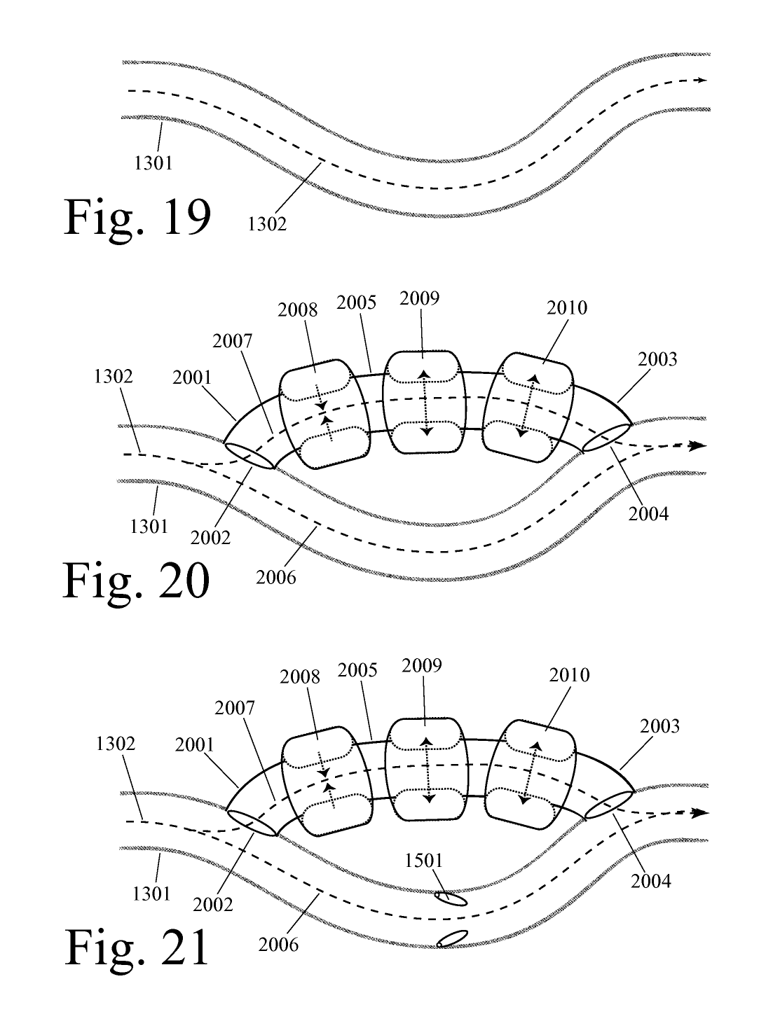

FIGS. 19 through 21 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

The example shown in FIGS. 19 through 21 is similar to the example shown in FIGS. 16 through 18 except that the blood flow increasing mechanism is now a peristaltic pump rather than a rotary pump. In this example, the blood flow increasing member moves blood by the sequential contraction and/or compression (from upstream to downstream) of a series of circumferential bands along the longitudinal axis of the implanted blood flow lumen. In an alternative example, a peristaltic pump can move blood by sequentially inflating and deflating a series of inflatable members (such as toroidal balloons) along the longitudinal axis (from upstream to downstream) of an implanted blood flow lumen.

FIG. 19 shows a view of the blood vessel before the device is implanted in order to show anatomical context for device implantation. FIG. 19 shows blood vessel 1301 and blood flow 1302 through this blood vessel. FIG. 20 shows this blood vessel after the device has been implanted. FIG. 20 shows the implanted blood flow lumen (further comprising an upstream lumen portion 2001, a middle lumen portion 2005, and a downstream lumen portion 2003) which is connected to blood vessel 1301 by upstream anastomosis 2002 and by downstream anastomosis 2004.

FIG. 20 also shows an implanted blood flow increasing mechanism which comprises a series of contractible and/or compressive circumferential bands (2008, 2009, and 2010) along the longitudinal axis of the implanted blood flow lumen. Sequential longitudinal contraction and/or compression of these contractible and/or compressive circumferential bands (2008, 2009, and 2010) causes blood to move longitudinally through the implanted blood flow lumen via peristalsis. In an alternative example, these contracting and/or compressing members do not have to span the full circumference of the lumen in order to provide peristaltic motion. In an alternative example, these contracting and/or compressing members need only span a portion of the circumference of the lumen. In an example, these contractible and/or compressing circumferential bands can each further comprise a control unit. In an example, these circumferential bands can have a common control unit. In an example, a control unit can further comprise a power source, an electric motor, hydraulic actuator, and/or pneumatic actuator. In an example, sequentially contracting and/or compressing members can be piezoelectric members. In an example, sequentially contracting and/or compressing bands can be pneumatic or hydraulic members.

In the example shown in FIG. 20, the implanted blood flow lumen causes a bifurcation of blood flow 1302. In this example, the portion of the blood flow which splits off into the implanted blood flow lumen is blood flow 2007 and the portion of the blood flow which continues through the blood vessel is blood flow 2006. In this example, the rotation of impeller 2008 increases blood flow 2007, which increases the combined blood flow 2006 and 2007 through the implanted blood flow lumen and the original blood vessel from an upstream location (anastomosis 2002) to a downstream location (anastomosis 2004). In an example, blood flow 2007 accelerates blood flow 2006 via entrainment when they reconverge at the downstream location. In this example, the post-implantation cross-sectional flow area available for blood to flow from an upstream location (anastomosis 2002) to a downstream location (anastomosis 2004) is not substantially less than the pre-implantation cross-sectional flow area available for blood flow between these locations in the original blood vessel alone, regardless of whether the blood flow increasing mechanism (impeller 2008) is operating or not. In this manner, this device does not hinder or restrict native blood flow when the blood flow increasing mechanism is not operating.

FIGS. 19 and 20 show an example of a device wherein: (a) a pre-implantation minimum cross-sectional flow area is the minimum cross-sectional flow area (from the upstream location to the downstream location) before the implanted blood flow lumen and the blood flow increasing mechanism are implanted; (b) a post-implantation minimum cross-sectional flow area is the minimum cross-sectional flow area (from the upstream location to the downstream location) which is unobstructed by the blood flow increasing mechanism when the blood flow increasing mechanism is not in operation after the implanted blood flow lumen and the blood flow increasing mechanism are implanted; and (c) the post-implantation minimum cross-sectional flow area is not substantially less than the pre-implantation minimum cross-sectional flow area. In an example, the definition of substantially less can be selected from the group consisting of: 5% less, 10% less, and 25% less.

FIGS. 19 and 20 also show an example of a device wherein post-implantation blood flow (from the upstream location to the downstream location) is greater than pre-implantation blood flow (from the upstream location to the downstream location) when the blood flow increasing mechanism is in operation transducing electromagnetic energy into kinetic energy. FIGS. 19 and 20 also show an example of a device wherein post-implantation blood flow (from the upstream location to the downstream location) when the blood flow increasing mechanism is not in operation is not substantially less than pre-implantation blood flow (from the upstream location to the downstream location). In an example, the definition of substantially less can be selected from the group consisting of: 5% less, 10% less, and 25% less.

FIGS. 19 and 20 also show an example of a device wherein an implanted blood flow lumen is configured to be implanted at least partially outside a blood vessel. In an example, the post-implantation minimum cross-sectional flow area can comprise the combined cross-sectional area through which blood flows unobstructed (from the upstream location to the downstream location) through either the implanted blood flow lumen or the blood vessel with which it is in fluid communication. FIG. 21 shows an example of a device like the one in FIG. 20 except that it also includes a one-way flow valve 1501. In this example, the one-way valve is implanted within the blood vessel.

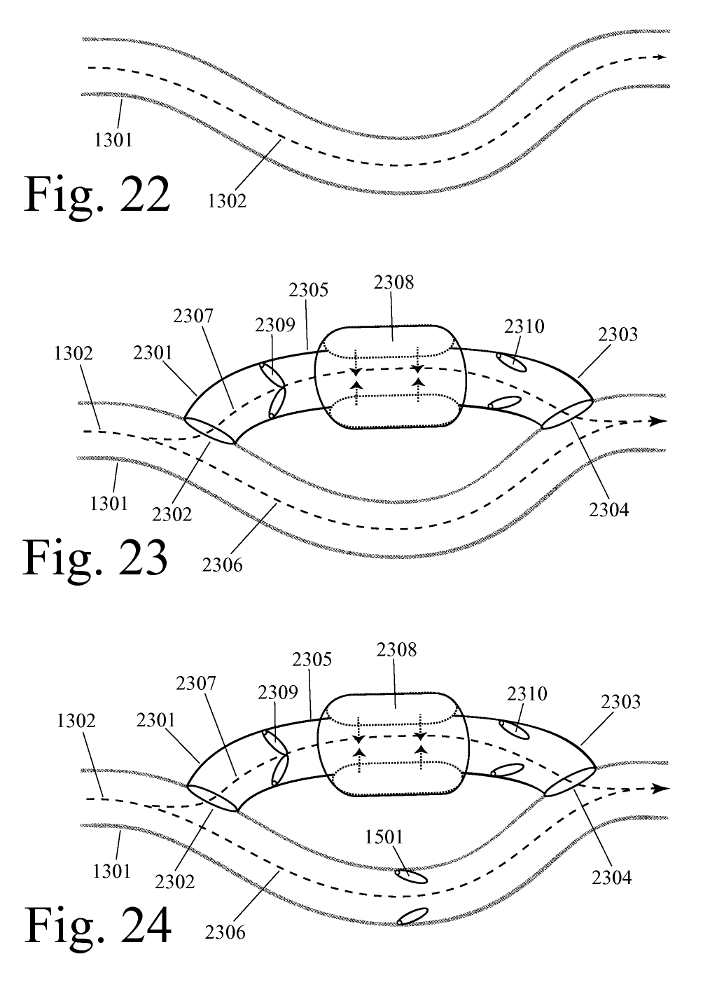

FIGS. 22 through 24 show another example of how this invention can be embodied in an implanted extracardiac device for supplementing blood circulation. The example shown in FIGS. 22 through 24 is similar to the example shown in FIGS. 19 through 21 except that the blood flow increasing mechanism now comprises a lumen-compressing member in combination with two one-way flow valves. FIG. 22 shows blood vessel 1301 and blood flow 1302 before the device is implanted. FIG. 23 shows this blood vessel after the device has been implanted. FIG. 23 shows the implanted blood flow lumen (further comprising an upstream lumen portion 2301, a middle lumen portion 2305, and a downstream lumen portion 2303) which is connected to blood vessel 1301 by upstream anastomosis 2302 and downstream anastomosis 2304. FIG. 23 also shows an implanted blood flow increasing mechanism which comprises lumen-compressing member 2308 and two one-way flow valves 2309 and 2310. In an example, member 2308 can further comprise a control unit with a power source, electric motor, hydraulic actuator, and/or pneumatic actuator.

In FIG. 23, the implanted blood flow lumen causes a bifurcation of blood flow 1302. In this example, the portion of the blood flow which splits off into the implanted blood flow lumen is blood flow 2307 and the portion of the blood flow which continues through the blood vessel is blood flow 2306. In this example, the rotation of impeller 2308 increases blood flow 2307, which increases the combined blood flow 2306 and 2307 through the implanted blood flow lumen and the original blood vessel from an upstream location (anastomosis 2302) to a downstream location (anastomosis 2304). In an example, blood flow 2307 accelerates blood flow 2306 via entrainment when they reconverge at the downstream location.

In this example, the post-implantation cross-sectional flow area available for blood to flow from an upstream location (anastomosis 2302) to a downstream location (anastomosis 2304) is not substantially less than the pre-implantation cross-sectional flow area available for blood flow between these locations in the original blood vessel alone, regardless of whether the blood flow increasing mechanism (impeller 2308) is operating or not. In this manner, this device does not hinder or restrict native blood flow when the blood flow increasing mechanism is not operating. FIGS. 22 and 23 also show an example of a device wherein an implanted blood flow lumen is configured to be implanted at least partially outside a blood vessel. FIG. 24 shows an example of a device like the one in FIG. 23 except that it also includes an additional one-way flow valve 1501 which is implanted within the blood vessel.

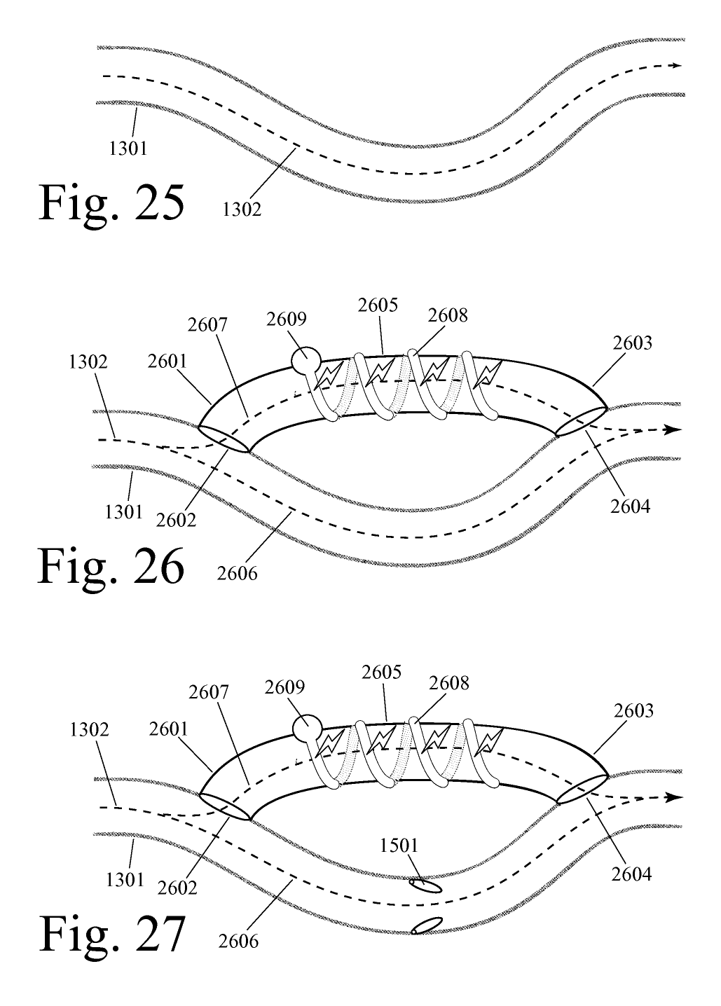

FIGS. 25 through 27 show another example of how this invention can be embodied in an implanted extracardiac device for supplementing blood circulation. This example is similar to the previous one except that now the blood flow increasing mechanism moves blood by electromagnetic interaction between (the ferrous components of) blood and an electromagnetic field. FIG. 25 shows blood vessel 1301 and blood flow 1302 before device implantation. FIG. 26 shows this blood vessel after device implantation. FIG. 26 shows the implanted blood flow lumen (further comprising an upstream lumen portion 2601, a middle lumen portion 2605, and a downstream lumen portion 2603) having been connected to blood vessel 1301 by upstream anastomosis 2602 and downstream anastomosis 2604. FIG. 26 also shows an implanted blood flow increasing mechanism comprising electromagnetic solenoid 2608 and control unit 2609. In an example, control unit 2609 can further comprise an electrical power source and can deliver electrical current through solenoid 2608 to create an electromagnetic field (symbolically represented here by lightning bolt symbols) which moves blood flow 2607.

In FIG. 26, the implanted blood flow lumen bifurcates blood flow 1302 into blood flow 2607 (which flows through the implanted blood flow lumen) and blood flow 2606 (which continues through the natural blood vessel) until these flows reconverge at the downstream location. In an example, blood flow 2607 accelerates blood flow 2606 via entrainment when they reconverge at the downstream location. In this example, post-implantation cross-sectional flow area is not substantially less than the pre-implantation cross-sectional flow area, regardless of whether the blood flow increasing mechanism is operating or not. In this manner, this device does not hinder or restrict native blood flow when the blood flow increasing mechanism is not operating. FIG. 27 shows an example of a device like the one in FIG. 26 except that it includes one-way flow valve 1501 which is implanted within the blood vessel.

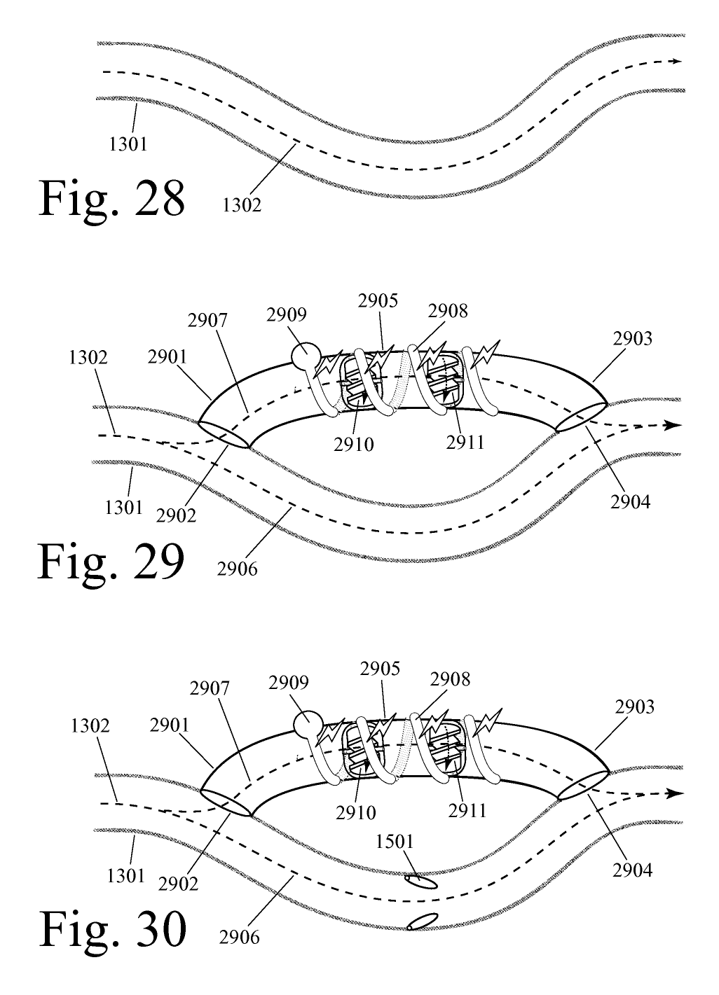

FIGS. 28 through 30 show another example of how this invention can be embodied in an implanted extracardiac device for supplementing blood circulation. This example is similar to the previous one except that now the blood flow increasing mechanism is an axial rotary pump whose impeller is rotated by electromagnetic interaction with an electromagnetic field. FIG. 28 shows blood vessel 1301 and blood flow 1302 before device implantation. FIG. 29 shows this blood vessel after device implantation. FIG. 29 shows the implanted blood flow lumen (further comprising an upstream lumen portion 2901, a middle lumen portion 2905, and a downstream lumen portion 2903) connected to blood vessel 1301 by upstream anastomosis 2902 and downstream anastomosis 2904. FIG. 29 also shows an implanted blood flow increasing mechanism comprising electromagnetic solenoid 2908, control unit 2909, and two axial impellers 2910 and 2911 which are rotated by electromagnetic interaction with the electromagnetic field which is created by solenoid 2908. In an example, control unit 2909 can further comprise an electrical power source and can deliver electrical current through solenoid 2908 in order to create the electromagnetic field (symbolically represented here by lightning bolt symbols) which moves impellers 2910 and 2911.

In FIG. 29, the implanted blood flow lumen bifurcates blood flow 1302 into blood flow 2907 and blood flow 2906, until these flows reconverge at the downstream location. Blood flow 2907 accelerates blood flow 2906 via entrainment when they reconverge. In this example, post-implantation cross-sectional flow area is not substantially less than pre-implantation cross-sectional flow area, regardless of whether the blood flow increasing mechanism is operating or not. In this manner, this device does not hinder or restrict native blood flow when the blood flow increasing mechanism is not operating. FIG. 30 shows an example of a device like the one in FIG. 29 except that it includes one-way flow valve 1501 which is implanted within the blood vessel.

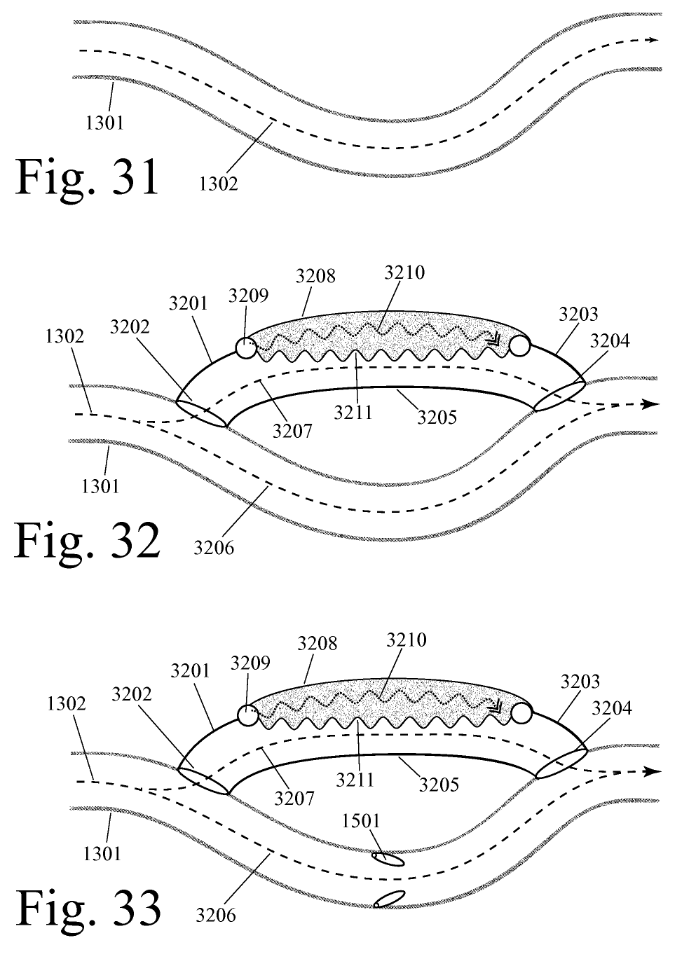

FIGS. 31 through 33 show another example of how this invention can be embodied in an implanted extracardiac device for supplementing blood circulation. This example is similar to the previous one except that now the blood flow increasing mechanism creates a longitudinally-travelling wave along a membrane (or other flexible surface) which is in fluid communication with blood within the implanted flow lumen. FIG. 31 shows blood vessel 1301 and blood flow 1302 before device implantation. FIG. 32 shows this blood vessel after device implantation. FIG. 32 shows the implanted blood flow lumen (further comprising an upstream lumen portion 3201, a middle lumen portion 3205, and a downstream lumen portion 3203) connected to blood vessel 1301 by upstream anastomosis 3202 and downstream anastomosis 3204.

FIG. 32 also shows an implanted blood flow increasing mechanism comprising fluid-filled elastic member 3208, elastic membrane 3211, and control unit 3209. In this example, control unit energizes a longitudinally-travelling (upstream to downstream) wave and/or pulse 3210 through fluid-filled elastic member 3208 which causes a longitudinally-travelling (upstream to downstream) wave along elastic member 3211. This longitudinally-travelling wave, in turn, frictionally engages blood to flow in an upstream to downstream direction. In this example, elastic member 3208 is a fluid-filled balloon. In an example, longitudinally-travelling wave and/pulse 3210 can be a pressure wave and/or pulse through the fluid in elastic member 3208. Control unit 3209 can further comprise a power source, a pressure pulse generator, and a wireless data transmitter/receiver.

In FIG. 32, an implanted blood flow lumen bifurcates blood flow 1332 into blood flows 3207 and 3206 until they reconverge. Blood flow 3207 can accelerate blood flow 3206 via entrainment when they reconverge. In this example, post-implantation cross-sectional flow area is not substantially less than pre-implantation cross-sectional flow area, regardless of whether the blood flow increasing mechanism is operating or not, so that this device does not hinder native blood flow when the blood flow increasing mechanism is not operating. FIG. 33 shows an example of a device like the one in FIG. 32 except that it includes one-way flow valve 1501 which is implanted within the blood vessel.

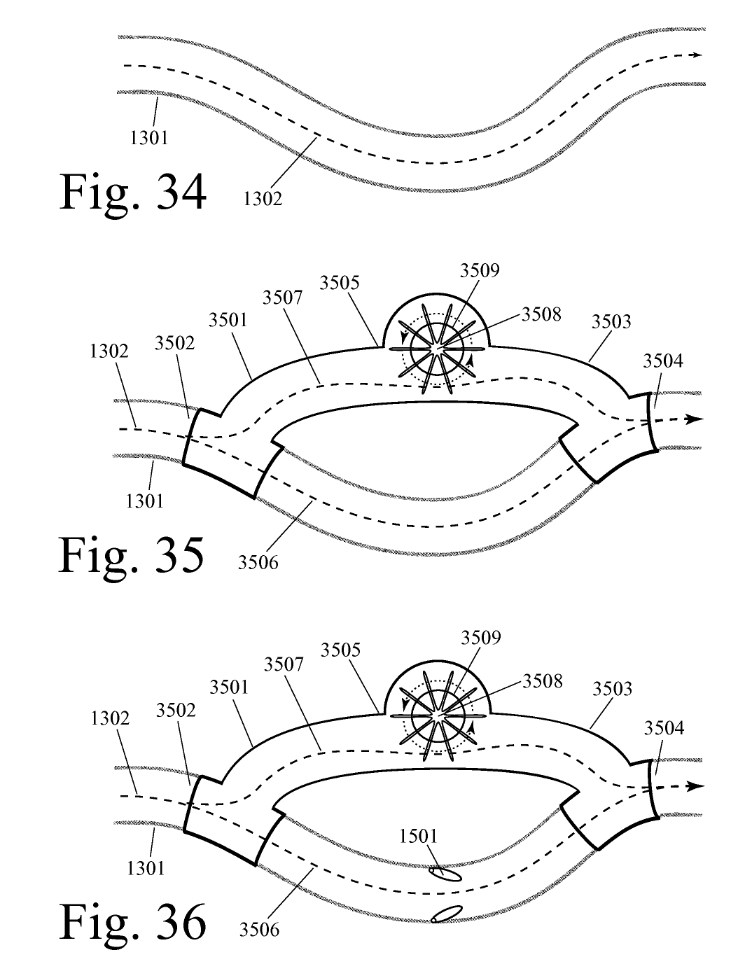

FIGS. 34 through 36 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

The example shown in FIGS. 34 through 36 is like the example shown in FIGS. 13 through 15, except that now the implanted blood flow lumen is connected to the blood vessel by two three-way connectors (or joints or branches) which are spliced into upstream and downstream locations instead of using two anastomoses. These figures show an example of how an implanted blood flow lumen can be implanted into fluid communication with a blood vessel using one or more connecting members or connection methods selected from the group consisting of: endovascular and/or transluminal insertion and expansion, surgical anastomosis, surgical sutures, purse string suture, drawstring, pull tie, friction fit, surgical staples, tissue adhesive, gel, fluid seal, chemical bonding, cauterization, blood vessel connector and/or joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

FIG. 34 shows the blood vessel before the device is implanted, including blood vessel 1301 and blood flow 1302. FIG. 35 shows this blood vessel after the device has been implanted. FIG. 35 shows an implanted blood flow lumen (comprising upstream lumen portion 3501, middle lumen portion 3505, and downstream lumen portion 3503) whose ends have been connected to blood vessel 1301 by two three-way connectors (or joints or branches) 3502 and 3504 which have been spliced into upstream and downstream locations along blood vessel 1301. FIG. 35 also shows an implanted blood flow increasing mechanism comprising rotating impeller 3508 as well as control unit 3509. In this example, control unit 3509 can further comprise a power source, an actuator, and a wireless data transmitter/receiver. In this example, impeller 3508 rotates around an axis which is substantially perpendicular to the longitudinal axis of the implanted blood flow lumen. In this example, this axis is also substantially perpendicular to the directional vector of blood flow through the implanted blood flow lumen.

In the example shown in FIG. 35, an implanted blood flow lumen causes a bifurcation of blood flow 1302. In this example, the portion of blood flow which is diverted into the implanted blood flow lumen is blood flow 3507 and the remaining portion of the blood flow which continues through the rest of the blood vessel is blood flow 3506. In this example, the rotation of impeller 3508 increases blood flow 3507, which increases combined blood flows 3506 and 3507 (through the implanted blood flow lumen and the original blood vessel) from the upstream location to the downstream location. In an example, blood flow 3507 accelerates blood flow 3506 via entrainment when they reconverge at the downstream location.

In the example shown in FIG. 35, the post-implantation cross-sectional flow area available for blood to flow from the upstream location to the downstream location is not substantially less than the pre-implantation cross-sectional flow area available for blood flow between these locations in the original blood vessel alone, regardless of whether the blood flow increasing mechanism (impeller 3508) is operating or not. In this design, this device does not hinder or restrict native blood flow when the blood flow increasing mechanism is not operating.

FIGS. 34 and 35 also show an example of a device wherein: (a) a pre-implantation minimum cross-sectional flow area is the minimum cross-sectional flow area (from the upstream location to the downstream location) before the implanted blood flow lumen and the blood flow increasing mechanism are implanted; (b) a post-implantation minimum cross-sectional flow area is the minimum cross-sectional flow area (from the upstream location to the downstream location) which is unobstructed by the blood flow increasing mechanism when the blood flow increasing mechanism is not in operation after the implanted blood flow lumen and the blood flow increasing mechanism are implanted; and (c) the post-implantation minimum cross-sectional flow area is not substantially less than the pre-implantation minimum cross-sectional flow area. In this example, the definition of substantially less can be selected from the group consisting of: 5% less, 10% less, and 25% less.

FIGS. 34 and 35 also show an example of a device wherein post-implantation blood flow (from an upstream location to a downstream location) is greater than pre-implantation blood flow (from the upstream location to the downstream location) when the blood flow increasing mechanism is in operation by transducing electromagnetic energy into kinetic energy. FIGS. 34 and 35 also show an example of a device wherein post-implantation blood flow (from the upstream location to the downstream location) when the blood flow increasing mechanism is not in operation is not substantially less than pre-implantation blood flow (from the upstream location to the downstream location). In an example, the definition of substantially less can be selected from the group consisting of: 5% less, 10% less, and 25% less.

FIGS. 34 and 35 also show an example of a device wherein an implanted blood flow lumen is configured to be implanted at least partially outside a blood vessel. In an example, the post-implantation minimum cross-sectional flow area can comprise the combined cross-sectional area through which blood flows unobstructed (from the upstream location to the downstream location) through either the implanted blood flow lumen or the blood vessel with which the lumen is in fluid communication. FIG. 35 also shows an example of a device wherein an implanted blood flow lumen is selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber. In particular, FIG. 35 shows an example of a device wherein an implanted blood flow lumen is an artificial vessel segment.

FIG. 35 also shows an example of a device wherein a blood flow increasing mechanism is selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump. In particular, FIG. 35 shows an example of a device wherein a blood flow increasing mechanism is an axial rotary pump.

FIG. 36 shows an example of a device that is like the one shown in FIG. 35 except that it further includes a one-way flow valve 1501. In this example, there is one such valve and it is configured to be inserted within the portion of the natural blood vessel between the upstream location and the downstream location.

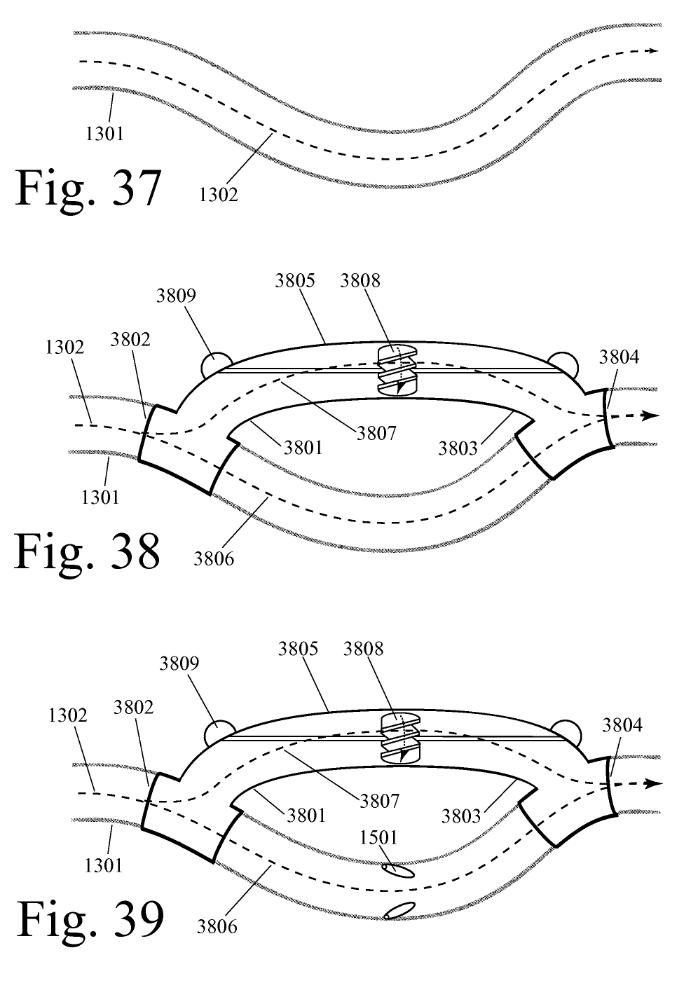

FIGS. 37 through 39 show another example of how this invention can be embodied in an implanted extracardiac device for supplementing blood circulation. This example is like the example shown in FIGS. 16 through 18, except that the implanted blood flow lumen is connected to the blood vessel by two three-way connectors (or joints or branches) which are spliced into upstream and downstream locations, instead of using two anastomoses. The example shown in FIGS. 37 through 39 comprises: upstream lumen portion 3801, middle lumen portion 3805, downstream lumen portion 3803, upstream three-way connector (or joint or branch) 3802, downstream three-way connector (or joint or branch) 3804, impeller 3808, control unit 3809, blood flows 3807 and 3806, and one-way flow valve 1501.

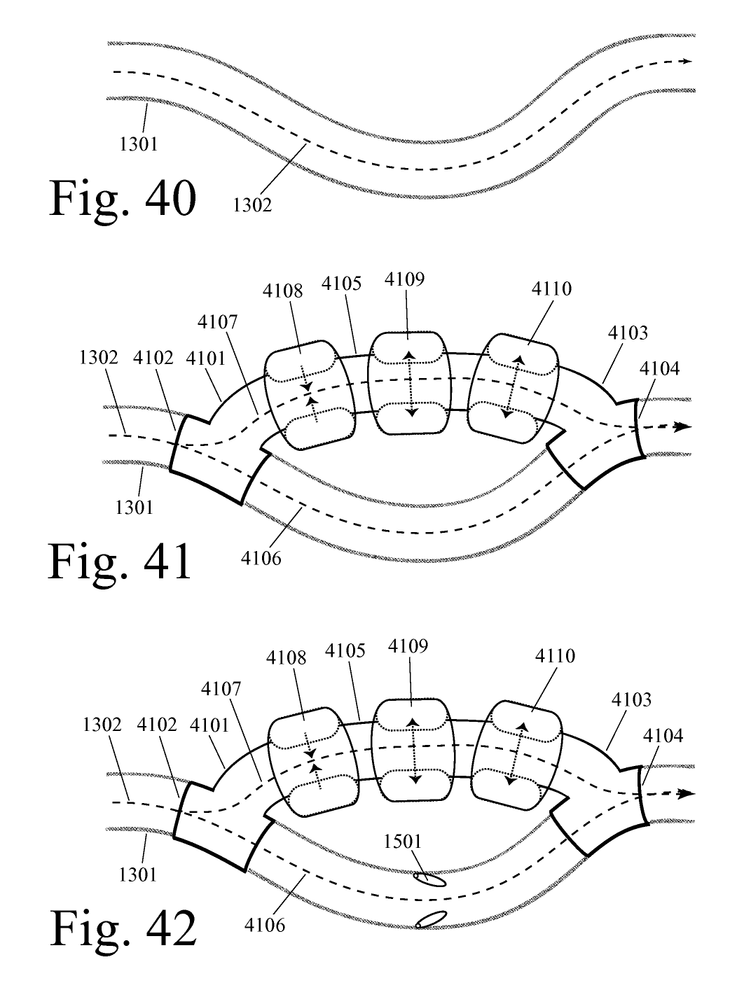

FIGS. 40 through 42 show another example of how this invention can be embodied in an implanted extracardiac device for supplementing blood circulation. This example is like the example shown in FIGS. 19 through 21, except that the implanted blood flow lumen is connected to the blood vessel by two three-way connectors (or joints or branches) which are spliced into upstream and downstream locations, instead of using two anastomoses. The example shown in FIGS. 40 through 42 comprises: upstream lumen portion 4101, middle lumen portion 4105, downstream lumen portion 4103, upstream three-way connector (or joint or branch) 4102, downstream three-way connector (or joint or branch) 4104, circumferential bands 4108, 4109, and 4110, blood flows 4107 and 4106, and one-way flow valve 1501.

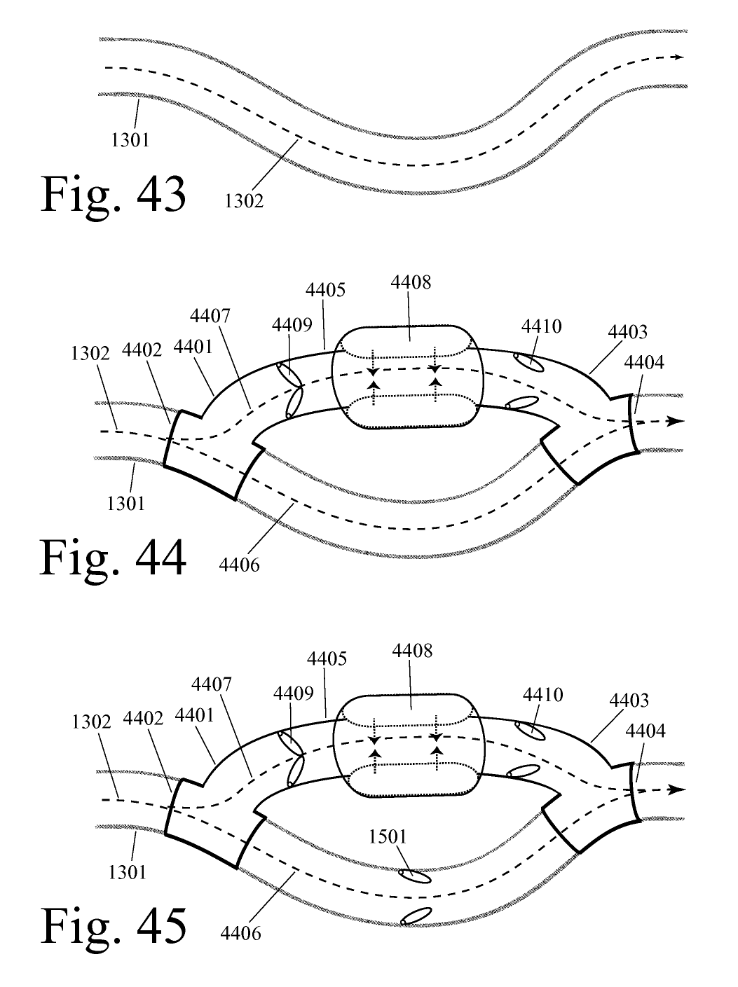

FIGS. 43 through 45 show another example of how this invention can be embodied in an implanted extracardiac device for supplementing blood circulation. This example is like the example shown in FIGS. 22 through 24, except that the implanted blood flow lumen is connected to the blood vessel by two three-way connectors (or joints or branches) which are spliced into upstream and downstream locations, instead of using two anastomoses. The example shown in FIGS. 43 through 45 comprises: upstream lumen portion 4401, middle lumen portion 4405, downstream lumen portion 4403, upstream three-way connector (or joint or branch) 4402, downstream three-way connector (or joint or branch) 4404, lumen-compressing member 4408, two one-way flow valves 4409 and 4410, blood flows 4407 and 4406, and one-way flow valve 1501.

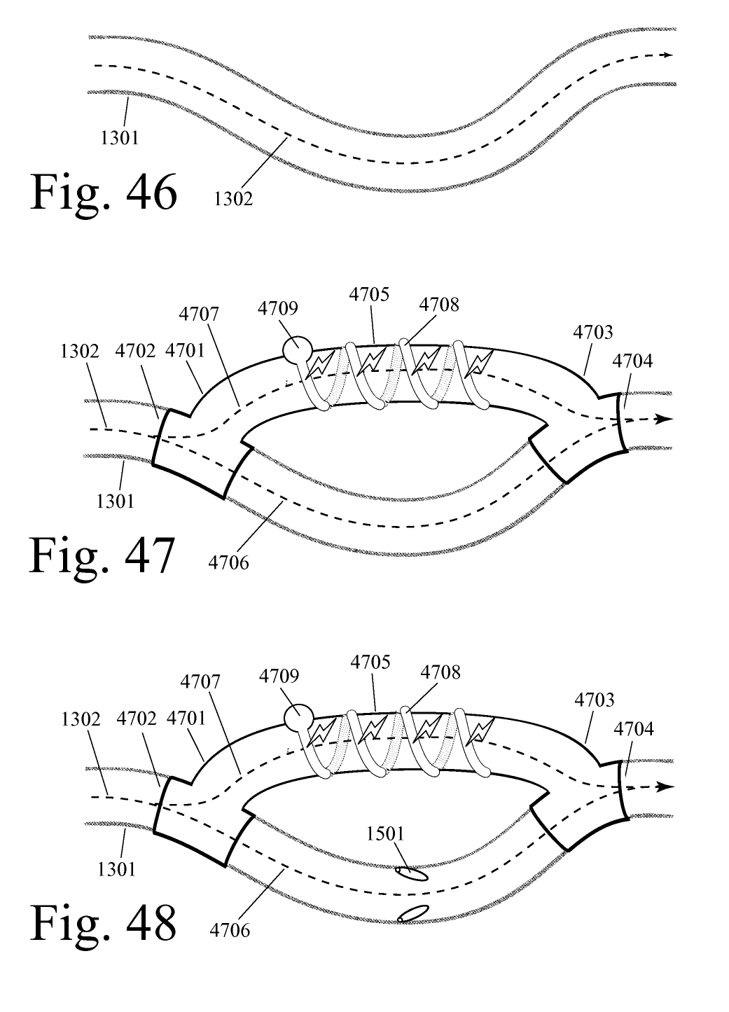

FIGS. 46 through 48 show another example of how this invention can be embodied in an implanted extracardiac device for supplementing blood circulation. This example is like the example shown in FIGS. 25 through 27, except that the implanted blood flow lumen is connected to the blood vessel by two three-way connectors (or joints or branches) which are spliced into upstream and downstream locations, instead of using two anastomoses. The example shown in FIGS. 46 through 48 comprises: upstream lumen portion 4701, middle lumen portion 4705, downstream lumen portion 4703, upstream three-way connector (or joint or branch) 4702, downstream three-way connector (or joint or branch) 4704, electromagnetic solenoid 4708, control unit 4709, blood flows 4707 and 4706, and one-way flow valve 1501.

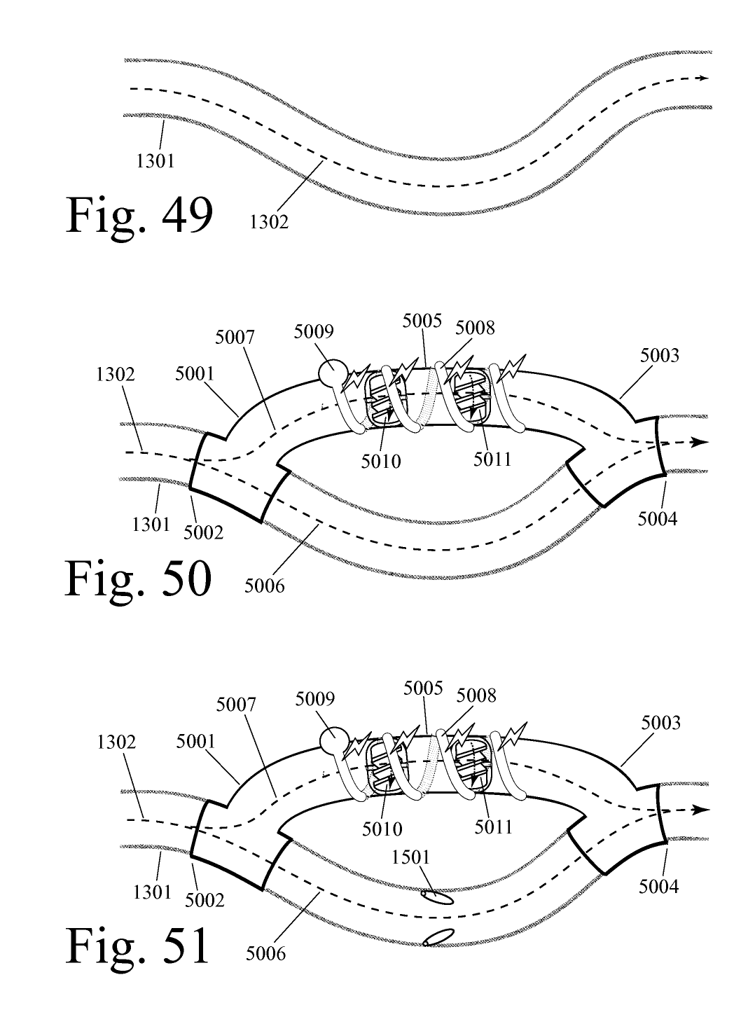

FIGS. 49 through 51 show another example of how this invention can be embodied in an implanted extracardiac device for supplementing blood circulation. This example is like the example shown in FIGS. 28 through 30, except that the implanted blood flow lumen is connected to the blood vessel by two three-way connectors (or joints or branches) which are spliced into upstream and downstream locations, instead of using two anastomoses. The example shown in FIGS. 49 through 51 comprises: upstream lumen portion 5001, middle lumen portion 5005, downstream lumen portion 5003, upstream three-way connector (or joint or branch) 5002, downstream three-way connector (or joint or branch) 5004, electromagnetic solenoid 5008, control unit 5009, two axial impellers 5010 and 5011, blood flows 5007 and 5006, and one-way flow valve 1501.

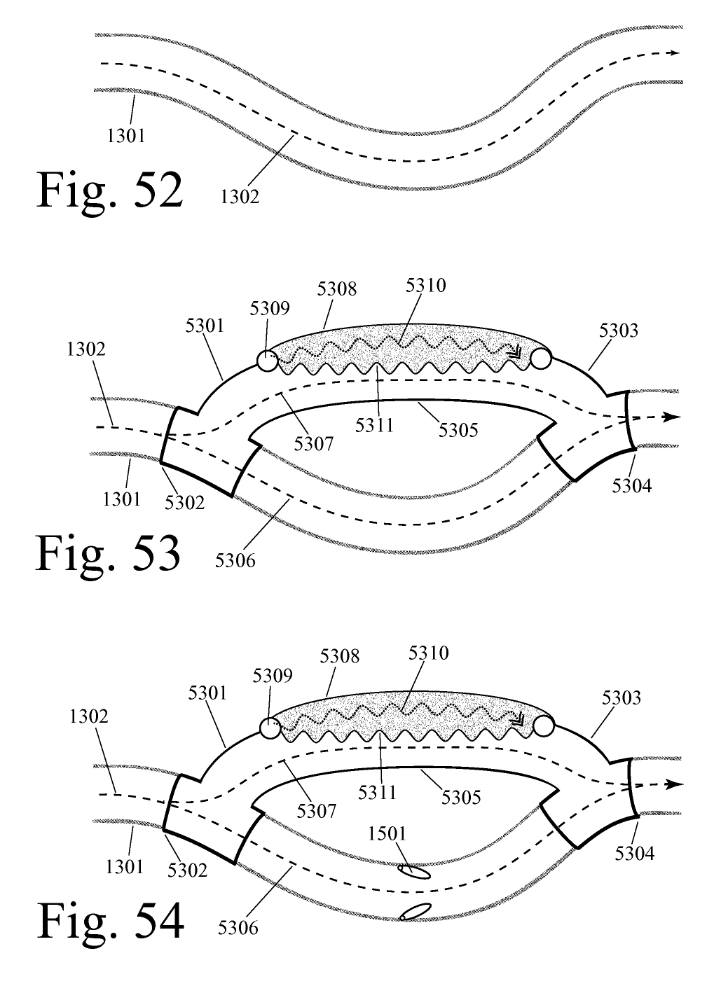

FIGS. 52 through 54 show another example of how this invention can be embodied in an implanted extracardiac device for supplementing blood circulation. This example is like the example shown in FIGS. 31 through 33, except that the implanted blood flow lumen is connected to the blood vessel by two three-way connectors (or joints or branches) which are spliced into upstream and downstream locations, instead of using two anastomoses. The example shown in FIGS. 52 through 54 comprises: upstream lumen portion 5301, middle lumen portion 5305, downstream lumen portion 5303, upstream three-way connector (or joint or branch) 5302, downstream three-way connector (or joint or branch) 5304, fluid-filled elastic member 5308, elastic membrane 5311, control unit 5309, wave and/or pulse 5310, blood flows 5307 and 5306, and one-way flow valve 1501.

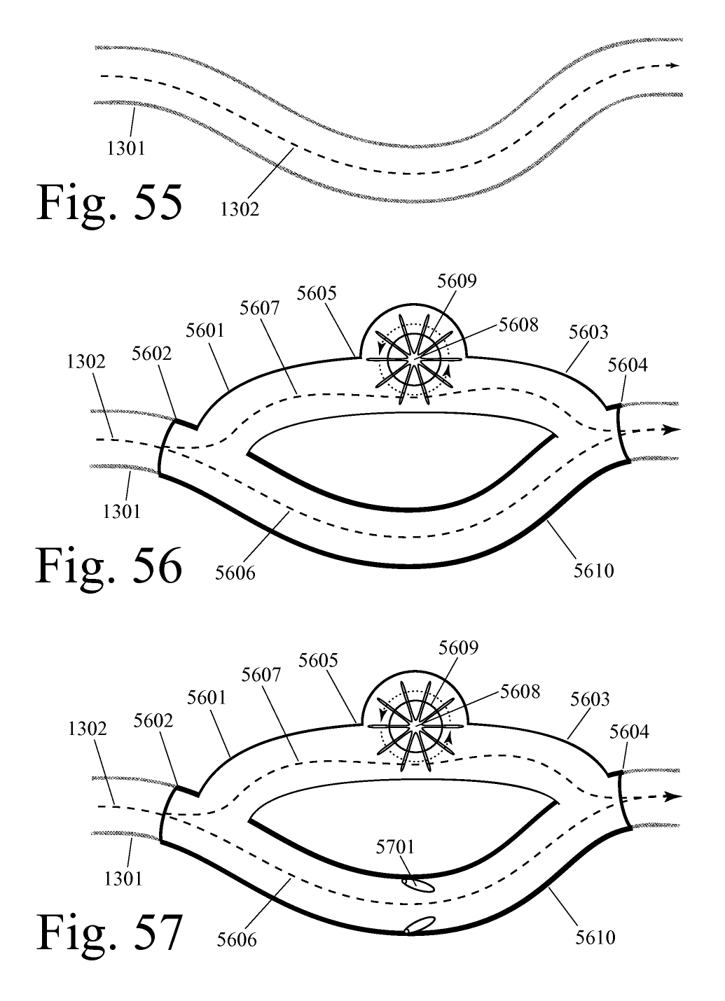

FIGS. 55 through 57 show another example of how this invention can be embodied in an implanted extracardiac device for supplementing blood circulation. This example is like the example shown in FIGS. 13 through 15, except that the implanted blood flow lumen is spliced into a natural blood vessel (from an upstream location to a downstream location) so as to entirely replace a longitudinal segment of the natural blood vessel. The example shown in FIGS. 55 through 57 comprises: upstream lumen portion 5601, middle lumen portion 5605, downstream lumen portion 5603, upstream splice connector 5602, downstream splice connector 5604, rotating impeller 5608, control unit 5609, blood flows 5607 and 5606, and one-way flow valve 1501.

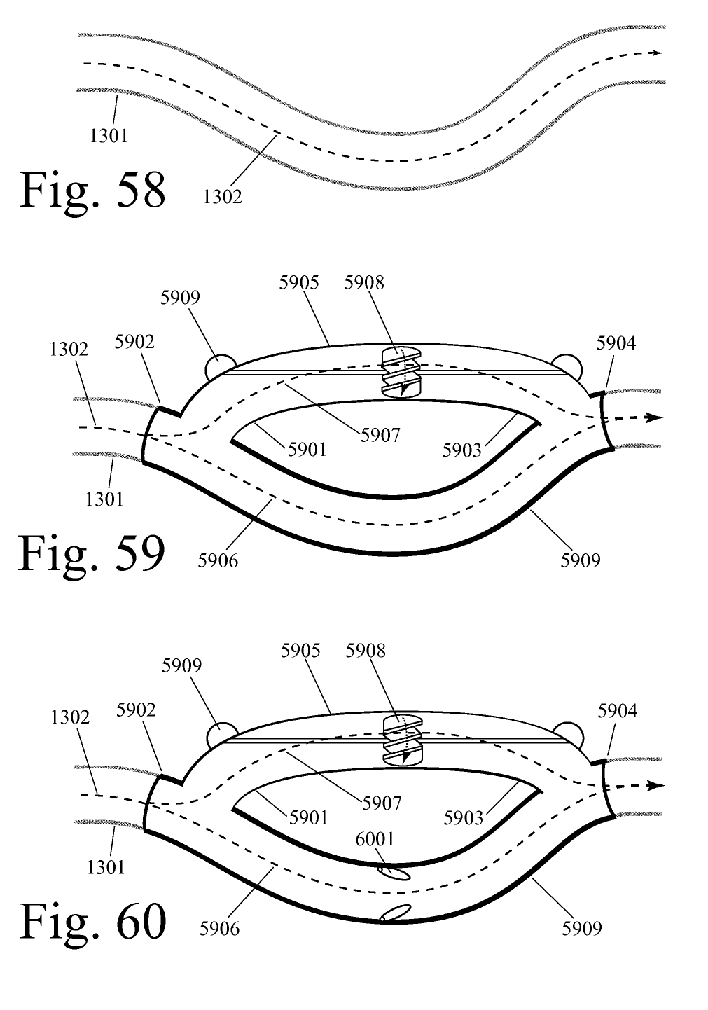

FIGS. 58 through 60 show another example of how this invention can be embodied in an implanted extracardiac device for supplementing blood circulation. This example is like the example shown in FIGS. 16 through 18, except that the implanted blood flow lumen is spliced into a natural blood vessel (from an upstream location to a downstream location) so as to entirely replace a longitudinal segment of the natural blood vessel. The example shown in FIGS. 58 through 60 comprises: upstream lumen portion 5901, middle lumen portion 5905, downstream lumen portion 5903, upstream splice connector 5902, downstream splice connector 5904, rotating impeller 5908, control unit 5909, blood flows 5907 and 5906, and one-way flow valve 1501.

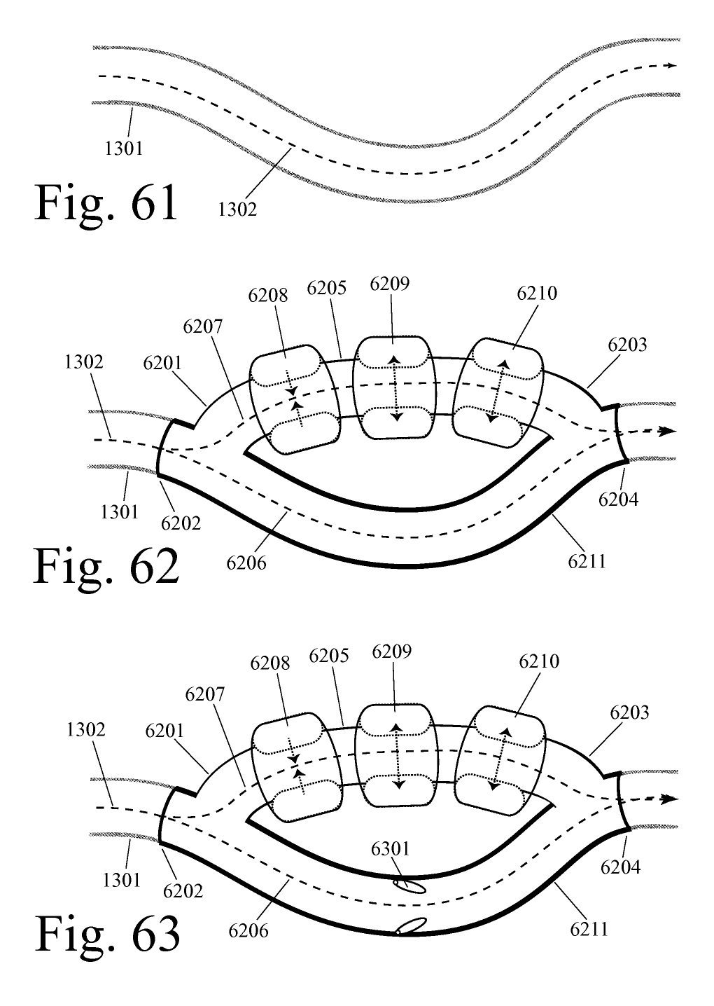

FIGS. 61 through 63 show another example of how this invention can be embodied in an implanted extracardiac device for supplementing blood circulation. This example is like the example shown in FIGS. 19 through 21, except that the implanted blood flow lumen is spliced into a natural blood vessel (from an upstream location to a downstream location) so as to entirely replace a longitudinal segment of the natural blood vessel. The example shown in FIGS. 61 through 63 comprises: upstream lumen portion 6201, middle lumen portion 6205, downstream lumen portion 6203, upstream splice connector 6202, downstream splice connector 6204, circumferential bands 6208, 6209, and 6210, blood flows 6207 and 6206, and one-way flow valve 1501.

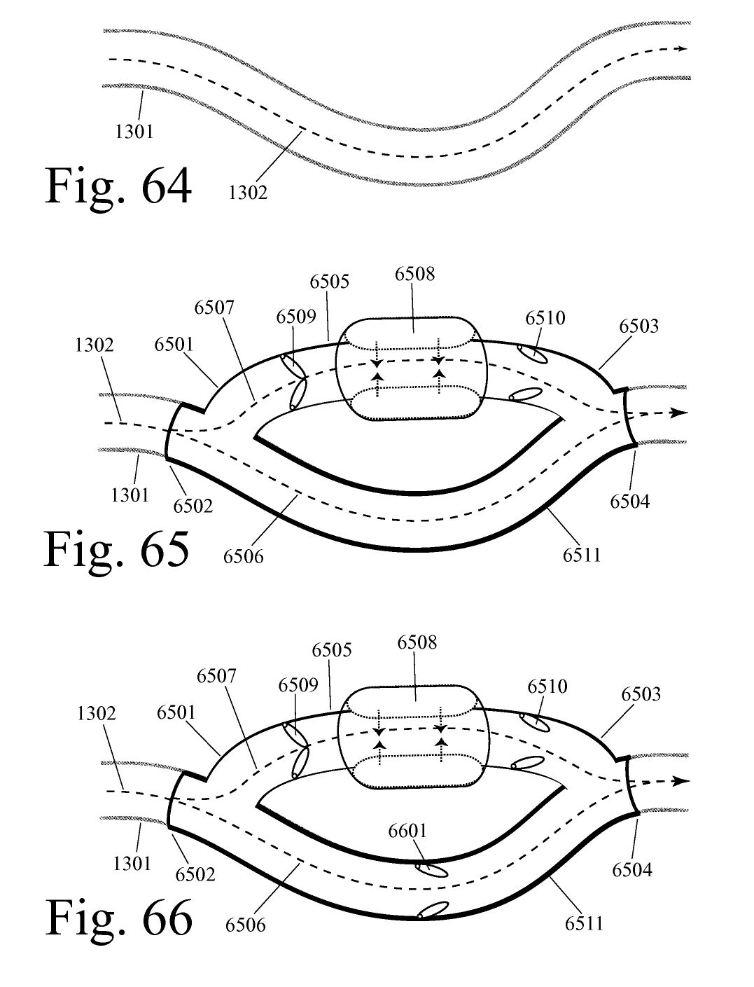

FIGS. 64 through 66 show another example of how this invention can be embodied in an implanted extracardiac device for supplementing blood circulation. This example is like the example shown in FIGS. 22 through 24, except that the implanted blood flow lumen is spliced into a natural blood vessel (from an upstream location to a downstream location) so as to entirely replace a longitudinal segment of the natural blood vessel. The example shown in FIGS. 64 through 66 comprises: upstream lumen portion 6501, middle lumen portion 6505, downstream lumen portion 6503, upstream splice connector 6502, downstream splice connector 6504, lumen-compressing member 6508, two one-way flow valves 6509 and 6510, blood flows 6507 and 6506, and one-way flow valve 1501.

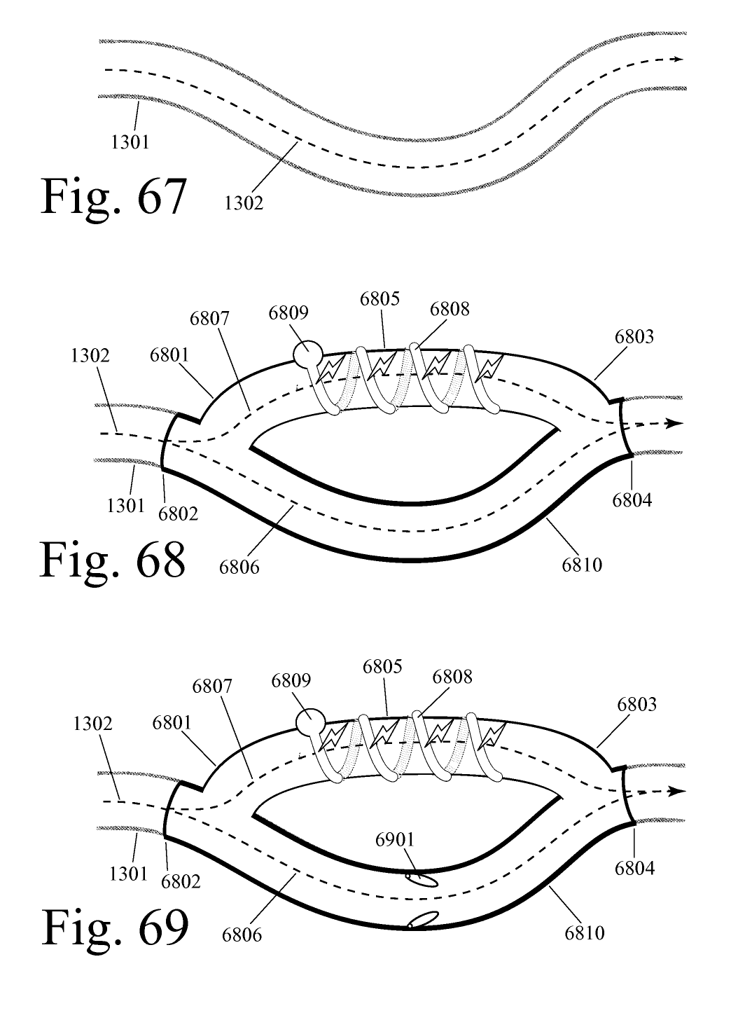

FIGS. 67 through 69 show another example of how this invention can be embodied in an implanted extracardiac device for supplementing blood circulation. This example is like the example shown in FIGS. 25 through 27, except that the implanted blood flow lumen is spliced into a natural blood vessel (from an upstream location to a downstream location) so as to entirely replace a longitudinal segment of the natural blood vessel. The example shown in FIGS. 67 through 69 comprises: upstream lumen portion 6801, middle lumen portion 6805, downstream lumen portion 6803, upstream splice connector 6802, downstream splice connector 6804, electromagnetic solenoid 6808, control unit 6809, blood flows 6807 and 6806, and one-way flow valve 1501.

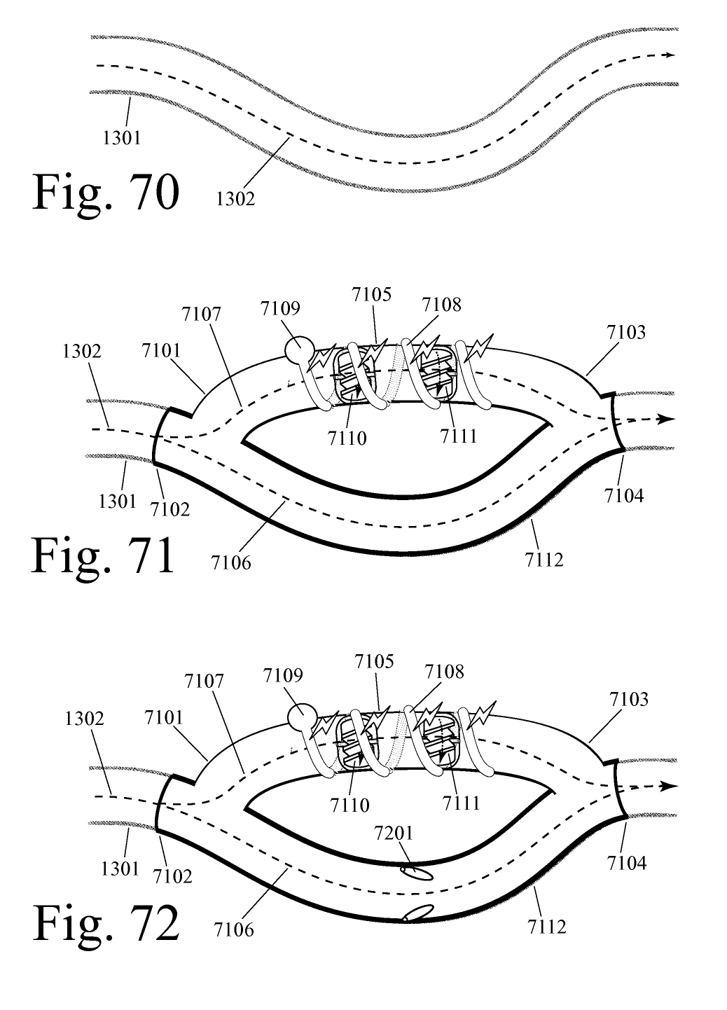

FIGS. 70 through 72 show another example of how this invention can be embodied in an implanted extracardiac device for supplementing blood circulation. This example is like the example shown in FIGS. 28 through 30, except that the implanted blood flow lumen is spliced into a natural blood vessel (from an upstream location to a downstream location) so as to entirely replace a longitudinal segment of the natural blood vessel. The example shown in FIGS. 70 through 72 comprises: upstream lumen portion 7101, middle lumen portion 7105, downstream lumen portion 7103, upstream splice connector 7102, downstream splice connector 7104, electromagnetic solenoid 7108, control unit 7109, two axial impellers 7110 and 7111, blood flows 7107 and 7106, and one-way flow valve 1501.

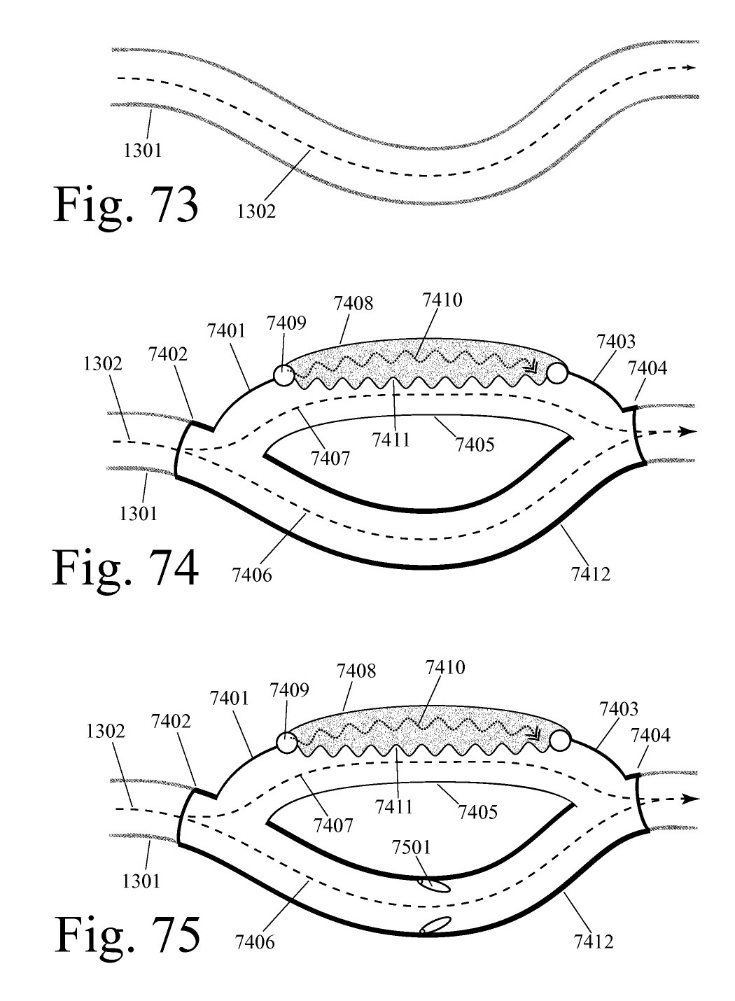

FIGS. 73 through 75 show another example of how this invention can be embodied in an implanted extracardiac device for supplementing blood circulation. This example is like the example shown in FIGS. 31 through 33, except that the implanted blood flow lumen is spliced into a natural blood vessel (from an upstream location to a downstream location) so as to entirely replace a longitudinal segment of the natural blood vessel. The example shown in FIGS. 73 through 75 comprises: upstream lumen portion 7401, middle lumen portion 7405, downstream lumen portion 7403, upstream splice connector 7402, downstream splice connector 7404, fluid-filled elastic member 7408, elastic membrane 7411, control unit 7409, wave and/or pulse 7410, blood flows 7407 and 7406, and one-way flow valve 1501.

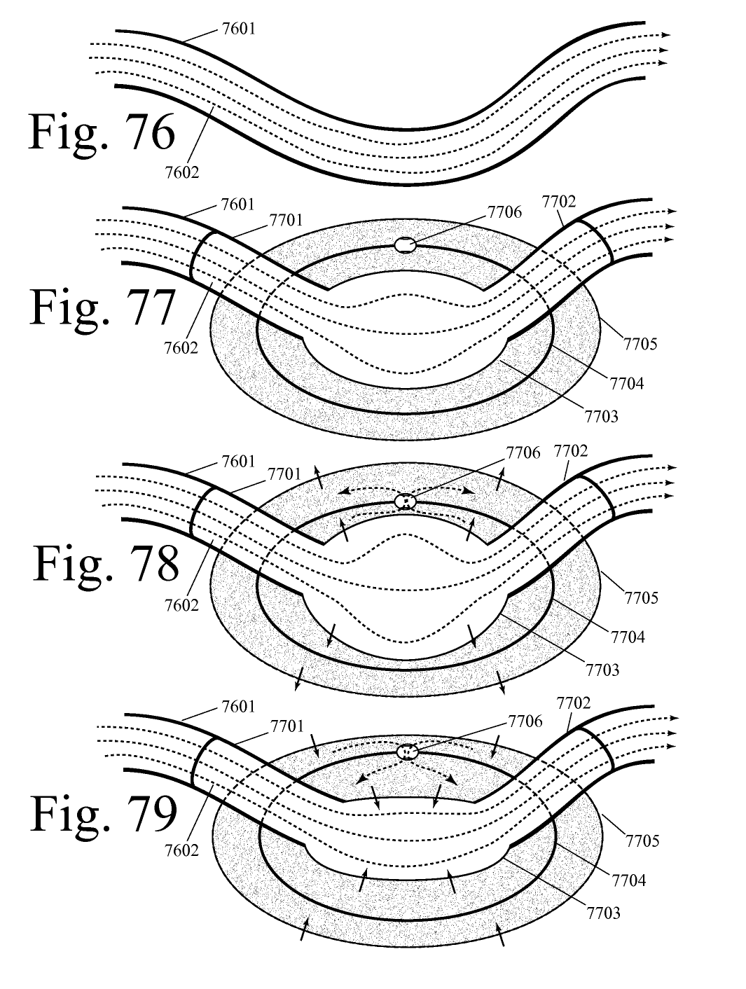

FIGS. 76 through 79 show an example of an implanted extracardiac circulatory assistance device without an active flow increasing mechanism which can enable automatic and/or remote adjustment of blood pressure level or variation. In an example, this can be embodied in an implanted device for adjustment of blood pressure level or variation comprising: (a) a first-layer member, wherein this first-layer member is configured to be in fluid communication with blood and wherein this first-layer member has a first elasticity level; (b) a second-layer member, wherein this second-layer member has a second elasticity level and the second elasticity level is less than the first elasticity level; (c) a flowable substance between the first-layer member and the second-layer member; (d) a third-layer member, wherein this third-layer member has a third elasticity level and the third elasticity level is greater than the second elasticity level; and (e) an adjustable-size opening through the second-layer member through which the flowable substance can flow, wherein size of this opening can be automatically and/or remotely adjusted. In an example, this device can further comprise a control unit with a power source, actuator, and wireless data transmitter/receiver which can automatically and/or remotely change the size of the opening.

FIG. 76 shows a blood vessel before implantation of the device. FIG. 77 shows this blood vessel and the device (after implantation) at a time when the first-layer member has a neutral configuration—neither very expanded nor very contracted. In an example, the first-layer member can have this neutral configuration when blood pressure is at a moderate level. This moderate level can be during a transitional point in the pulsation cycle or a long-term moderate level.

FIG. 78 shows this blood vessel and the device at a time when the first-layer member has an expanded configuration. In an example, the first-layer member can have this expanded configuration when blood pressure is at a high level and the adjustable-size opening is at least partially open. If the adjustable-size opening were completely closed, then the first-layer member would be constrained by the counter-pressure of the flowable substance and would not be able to expand. In an example, the first-layer member can have an expanded configuration when blood pressure is at a high level. This high level can be during a peak point in the pulsation cycle or reflect long-term hypertension.

FIG. 79 shows this blood vessel and the device at a time when the first-layer member has a contracted configuration. In an example, the first-layer member can have this contracted configuration when blood pressure is at a low level and the adjustable-size opening is at least partially open. If the adjustable-size opening were completely closed, then the first-layer member would be constrained by the vacuum effect of the flowable substance and would not be able to contract. In an example, the first-layer member can have a contracted configuration when blood pressure is at a low level. This low level can be during a nadir in the pulsation cycle or reflect long-term hypotension.

With respect to individual components, FIGS. 77 through 79 show: blood vessel 7601, blood flow 7602, upstream connector 7701, downstream connector 7702, first-layer member 7703, second-layer member 7704, third-layer member 7705, and adjustable-size opening 7706. In an example, this device can further comprise a control unit, actuator, and wireless data transmitter/receiver for automatic and/or remote adjustment of the size of adjustable-size opening 7706. In an example, the second-layer member can at least partially surround the first-layer member. In an example, the first-layer member, second-layer member, and third-layer member can be nested. In an example, the first-layer member, second-layer member, and third-layer member can be circumferentially nested. In an example, the first-layer member, second-layer member, and third-layer member can be substantially concentric. In an example, the first-layer member and third-layer member can be balloons. In an example, the second-layer member can be a relatively rigid structure.

In an example, the flowable substance can be between the second-layer member and the third-layer member as well as between the first-layer member and the second-layer member. In an example, there can be multiple adjustable-size openings through which the flowable substance can flow through the second-layer member. In an example, there can be multiple openings through which the flowable substance can flow through the second-layer member and the proportion of these openings which are open or closed can be adjusted. In an example, adjustment of the size of one or more openings can be done with a piezoelectric member. In an example, adjustment of the size of one or more openings can be done with a MEMS actuator or other microscale actuator.

In an example, when the size of an opening is increased then the first-layer member expands more freely in response to increases in blood pressure and when the size of the opening is decreased then the first-layer member expands less freely in response to increases in blood pressure. In an example, when the size of an opening is increased then greater expansion or contraction of the first-layer member causes less variation in blood pressure and when the size of the opening is decreased then lesser expansion or contraction of the first-layer member causes greater variation in blood pressure. This variation in blood pressure can be variation in pressure within the pulsation cycle or longer-term variation in blood pressure. In an example, increasing the size of the opening causes a decrease in blood pressure and decreasing the size of the opening causes an increase in blood pressure. In an example, increasing the size of the opening causes a decrease in blood pressure variation and decreasing the size of the opening causes an increase in blood pressure variation. In an example, the flowable substance can be a fluid. In an example, the flowable substance can be a gas.

In an example, this device can function as a blood reservoir with adjustable elasticity. In an example, the elasticity of a blood reservoir can be automatically adjusted based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, the elasticity of a blood reservoir can be automatically adjusted based on data from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, a plurality of such devices can be implanted in different peripheral blood vessels to create a coordinated system of variable-elasticity blood reservoirs which can be used to adjust and control the level and/or variation of a person's blood pressure. In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface. In an example, a plurality of such devices can be implanted in multiple locations in a person's peripheral blood vessels in order to create a system of distributed circulatory assistance which therapeutically reduces the workload of the heart without harming cardiac tissue.

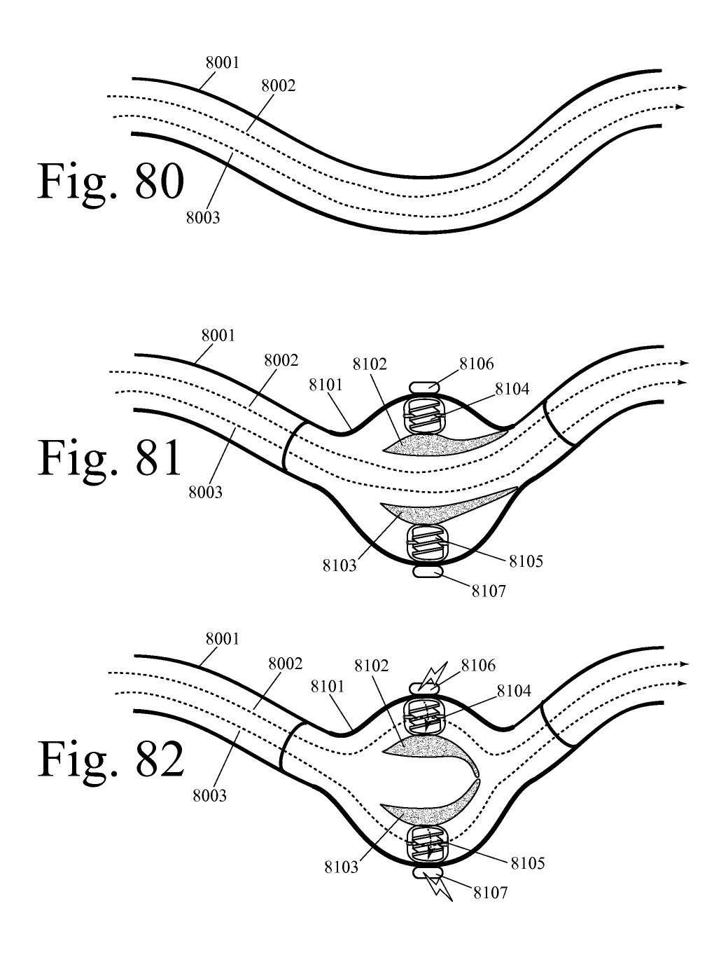

FIGS. 80 through 82 show an example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 80 shows a blood vessel before implantation to show anatomical context. FIG. 81 shows the device after implantation at a time when a blood flow increasing mechanism is not in operation. At this time, there is only native blood flow. FIG. 82 shows the device at a time when the blood flow increasing mechanism is in operation. An important feature of this design is that the device increases blood flow when the blood flow increasing mechanism is in operation, but does not hinder native blood flow when the blood flow increasing mechanism is not in operation. Specifically, FIGS. 80 through 82 show: blood vessel 8001, blood flows 8002 and 8003, implanted blood flow lumen 8101, first flow valve 8102, second flow valve 8103, first impeller 8104, second impeller 8105, first control unit 8106, and second control unit 8107. In an example, a control unit can further comprise a power source, an actuator, and wireless data transmitter/receiver.

In this example, implanted blood flow lumen 8101 is spliced into blood vessel 8001 so as to completely replace a longitudinal segment of the blood vessel. In this example, implanted blood flow lumen 8101 has an arcuate non-uniform cross-sectional shape. In this example, implanted blood flow lumen 8101 is bulbous. In this example, implanted blood flow lumen 8101 has multiple flow channels running through it. In this example, implanted blood flow lumen 8101 has a first (upper) flow channel, a second (lower) flow channel, and third (middle) flow channel. In this example, first impeller 8104 is in fluid communication with the first (upper) flow channel and second impeller 8105 is in fluid communication with the second (lower) flow channel. In this example, first impeller 8104 accelerates blood flow through the first (upper) flow channel when it is in operation and second impeller 8105 accelerates blood flow through the second (lower) flow channel when it is in operation. In this example, the third (middle) flow channel has a cross-sectional flow area which is not less than the cross-sectional flow area of longitudinal segment of the natural blood vessel which was replaced. In this manner, this device does not hinder native blood flow (relative to pre-implantation native flow) when impellers 8104 and 8105 are not in operation.

FIG. 81 shows this device at a time when the blood flow increasing mechanism is not in operation. At this time, neither impeller 8104 nor impeller 8105 are rotating. In this example, the downstream flaps of first and second flow valves 8102 and 8103 are flexible. In this figure, native blood flow pushes against the flexible downstream flaps of first and second flow valves, 8102 and 8103, thereby pushing these valves out of the third (middle) flow channel so that native blood flow is not hindered.

FIG. 82 shows this device at a time when the blood flow increasing mechanism is in operation transducing electrical energy (from an electrical power source) into kinetic energy (in the form of blood flow). In this figure, impellers 8104 and 8105 are both rotating. This rotation accelerates blood flows 8002 and 8003. In this example, blood flows 8002 and 8003 are sufficiently strong relative to native blood flow that they push flow valves 8102 and 8102 together, which prevents reverse flow through the third (middle) flow channel. However, flow valves can remain open even when the flow increasing mechanism is operating if native blood flow is sufficiently strong and/or if the supplemental flow increases are sufficiently modest. When native blood flow is sufficiently strong and/or blood flows 8002 and 8003 are not as strong relative to native blood flow, then the flow valves will remain at least partially open. This can allow all three blood flows (native flow and both accelerated flows) to flow simultaneously. This design enables this device to provide truly supplementing, not supplanting, circulatory support for therapeutic benefit.

In the example shown in FIGS. 80 through 82, a blood flow increasing mechanism comprises two axial rotary blood pumps. In an example, this design can include more than two blood pumps and more than three blood flow channels. In other examples, one or more blood flow increasing mechanisms for use in this design can be selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump.

In an example, control units 8106 and 8107 can control and adjust the operation of impellers 8104 and 8105 based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control units 8106 and 8107 can control and adjust the operation of impellers 8104 and 8105 based on data from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit.

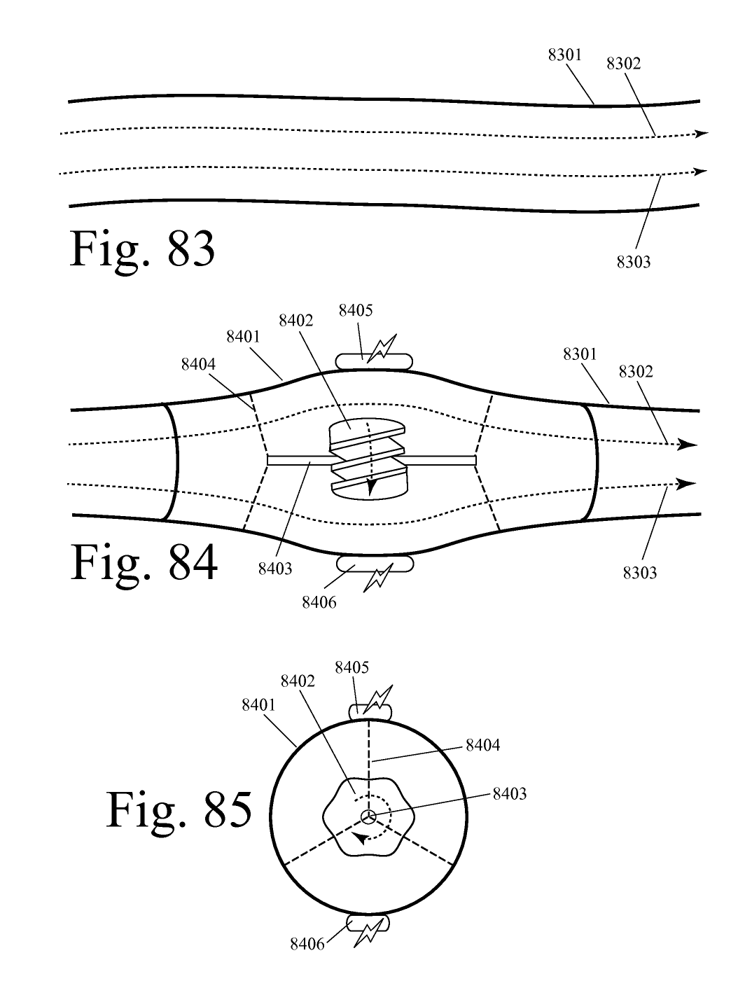

FIGS. 83 through 85 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 83 shows a blood vessel before implantation. FIG. 84 shows a longitudinal semi-transparent view of the device after implantation. FIG. 85 shows a lateral cross-sectional view of the device after implantation. FIGS. 83 through 85 show: blood vessel 8301, blood flows 8302 and 8303, implanted blood flow lumen 8401, impeller 8402, axle 8403, struts (including 8404), and control units 8405 and 8406. In this example, impeller 8402 rotates around axle 8403. Axle 8403 is held in a central position (substantially coaxial with implanted blood flow lumen 8401) by struts (including 8404). In this example, impeller 8402 is rotated by magnetic interaction with an electromagnetic field which is generated by control units 8405 and 8406. In another example, impeller 8402 can be rotated by a direct mechanical drive mechanism.

In this example, implanted blood flow lumen 8401 is spliced into a blood vessel 8301 so as to completely replace a longitudinal segment of the blood vessel. In this example, implanted blood flow lumen 8401 has an arcuate non-uniform cross-sectional shape. In this example, implanted blood flow lumen 8401 is bulbous. In this example, the minimum net cross-sectional blood flow area of blood flow lumen 8401 after subtracting out the cross-sectional area which is obstructed by impeller 8402 is still greater than the minimum cross-sectional blood flow area of the longitudinal segment of blood vessel 8301 which was replaced. In this manner, this device increases blood flow when the blood flow increasing mechanism is in operation, but does not hinder native blood flow when the blood flow increasing mechanism is not in operation.

In an example, control units 8405 and 8406 can control and adjust the operation of impeller 8402 based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control units 8405 and 8406 can control and adjust the operation of impeller 8402 based on data from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface. In an example, a plurality of such devices can be implanted in multiple locations in a person's peripheral blood vessels in order to create a system of distributed circulatory assistance which therapeutically reduces the workload of the heart without harming cardiac tissue.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit.

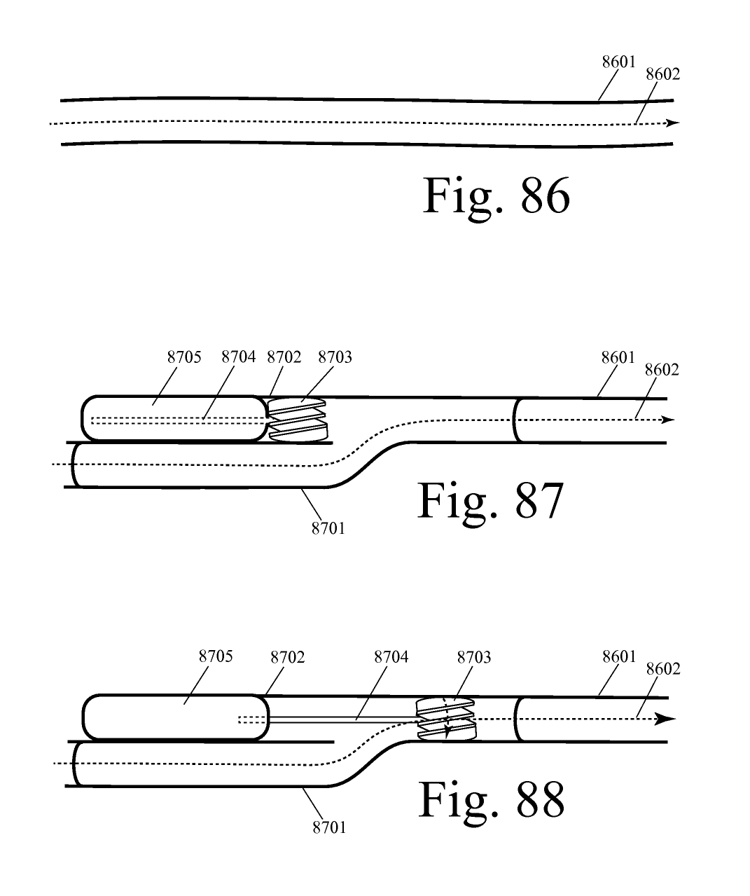

FIGS. 86 through 88 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 86 shows a blood vessel before implantation. FIG. 87 shows a longitudinal semi-transparent view of the device (after implantation) at a time when the blood flow increasing mechanism is not in operation. FIG. 88 shows a longitudinal semi-transparent view of the device at a time when the blood flow increasing mechanism is in operation. FIGS. 86 through 88 show: blood vessel 8601, blood flow 8602, implanted blood flow lumen 8701 with branching lumen portion 8702, impeller 8703, axle 8704, and control unit 8705. Control unit 8705 can further comprises a power source, an actuator which can move axle 8704 longitudinally (in and out) as well as rotationally, and a wireless data transmitter/receiver.

In this example, implanted blood flow lumen 8701 has been spliced into a blood vessel 8601 so as to completely replace a longitudinal segment of the blood vessel. In this example, implanted blood flow lumen is arcuate with a branching lumen portion (8702). In this example, the branching lumen portion is substantially parallel to the primary lumen of the implanted blood flow lumen. As shown in FIG. 87, when the blood flow increasing mechanism is not in operation, then axle 8704 is longitudinally retracted into control unit 8705 so that impeller 8703 is not within the primary lumen of the implanted blood flow lumen and does not obstruct native blood flow through the blood flow lumen.

As shown in FIG. 88, when the blood flow increasing mechanism is in operation, then axle 8704 is longitudinally extended out from control unit 8705 so that impeller 8703 is in the primary lumen of the implanted blood flow lumen, wherein the impeller engages and accelerates blood flow 8602 through the blood flow lumen. In this manner, this device increases blood flow when the blood flow increasing mechanism is in operation, but does not hinder native blood flow when the blood flow increasing mechanism is not in operation. In an example, axle 8704 can be moved longitudinally (in or out) by a hydraulic mechanism within control unit 8705. In an example, axle 8704 can be moved longitudinally (in or out) by an electromagnetic actuator within control unit 8705.

FIGS. 86 through 88 show an example of how this invention can be embodied in a device wherein: pre-implantation minimum cross-sectional flow area is the minimum cross-sectional flow area from the upstream location to the downstream location before the implanted blood flow lumen and the blood flow increasing mechanism are implanted; post-implantation minimum cross-sectional flow area is the minimum cross-sectional flow area from the upstream location to the downstream location which is unobstructed by the blood flow increasing mechanism when the blood flow increasing mechanism is not in operation after the implanted blood flow lumen and the blood flow increasing mechanism are implanted; and post-implantation minimum cross-sectional flow area is not substantially less than the pre-implantation minimum cross-sectional flow area.

FIGS. 86 through 88 also show an example of how this invention can be embodied in a device wherein: post-implantation blood flow from the upstream location to the downstream location is greater than pre-implantation blood flow from the upstream location to the downstream location when the blood flow increasing mechanism is in operation transducing electromagnetic energy into kinetic energy; and wherein post-implantation blood flow from the upstream location to the downstream location when the blood flow increasing mechanism is not in operation is not substantially less than pre-implantation blood flow from the upstream location to the downstream location

In example variations, an implanted blood flow lumen can be implanted into fluid communication with a blood vessel by one or more connecting members or connection methods which are selected from the group consisting of: endovascular insertion and expansion within a blood vessel, anastomosis, sutures, purse string suture, drawstring, pull tie, friction fit, surgical staples, tissue adhesive, gel, fluid seal, biochemical bond, cauterization, (three-way) vessel joint, vessel branch, twist connector, helical threads or screw connector, connection port, interlocking joints, tongue and groove connection, flanged connector, beveled ridge, magnetic connection, plug connector, circumferential ring, inflatable ring, and snap connector. In example variations, an implanted blood flow lumen can be selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber.

FIGS. 86 through 88 also show an example of how this invention can be embodied in a device wherein a blood flow increasing mechanism has a first configuration (retracted axle 8704 and impeller 8703) when it is not in operation transducing electromagnetic energy into kinetic energy, wherein the blood flow increasing mechanism has a second configuration (extended axle 8704 and impeller 8703) when it is in operation transducing electromagnetic energy into kinetic energy, and wherein the second configuration occupies a larger portion of the post-implantation minimum cross-sectional flow area than the first configuration. This device also shows how the post-implantation minimum cross-sectional flow area can be substantially less than the pre-implantation minimum cross-sectional flow area when the blood flow increasing mechanism is in the second configuration, but not when the blood flow increasing mechanism is in the first configuration.

FIGS. 86 through 88 also show an example of how this invention can be embodied in a device wherein a blood flow increasing mechanism is moved from the first configuration to the second configuration (longitudinal extension of axle 8704) by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

In an example, control unit 8705 can control and adjust the operation of axle 8704 and impeller 8703 based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control unit 8705 can control and adjust the operation of axle 8704 and impeller 8703 based on data from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface. In an example, a plurality of such devices can be implanted in multiple locations in a person's peripheral blood vessels in order to create a system of distributed circulatory assistance which therapeutically reduces the workload of the heart without harming cardiac tissue.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit.

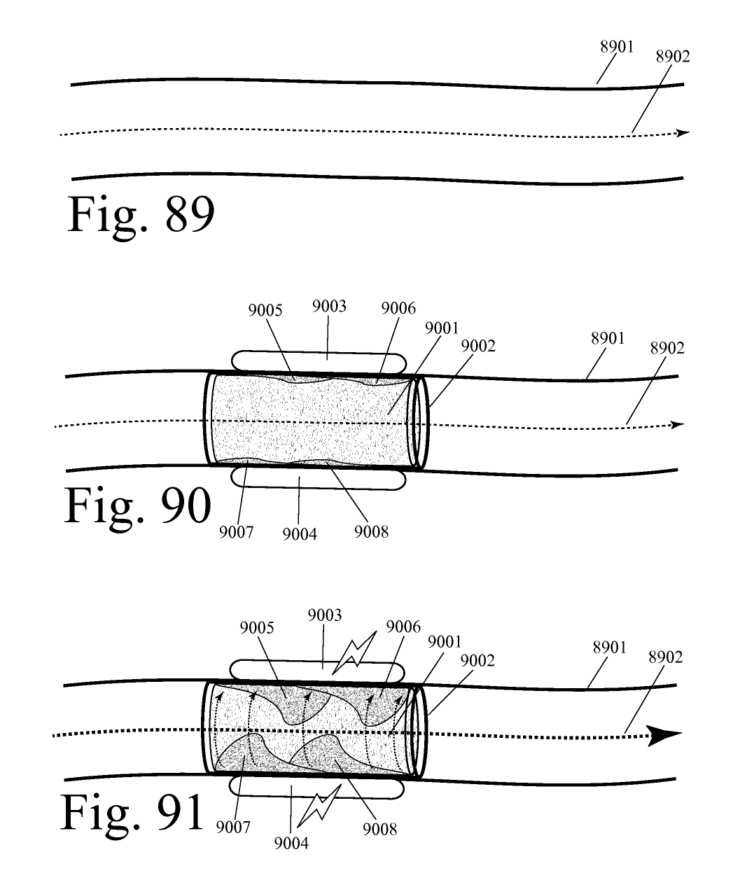

FIGS. 89 through 91 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 89 shows a blood vessel before implantation. FIG. 90 shows a longitudinal semi-transparent view of the device at a time when the blood flow increasing mechanism is not in operation. FIG. 91 shows a longitudinal semi-transparent view of the device at a time when the blood flow increasing mechanism is in operation. FIGS. 89 through 91 show: blood vessel 8901, blood flow 8902, implanted blood flow lumen 9002, rotating cylinder 9001, extendable fins (including 9005, 9006, 9007, and 9008), and control units 9003 and 9004. In an example, the control units can further comprise a power source, an actuator, an electromagnetic field generator, and a wireless data transmitter/receiver.

In an example, the extendable members can be more-generally selected from the group consisting of: fins, vanes, blades, airfoils, winglets, helical structures, and strips. In an example, the rotating cylinder and a plurality of extendable fins (or other extendable members) can together comprise an impeller. In an example, a plurality of extendable fins can together comprise a helical structure when they are extended outwards from the walls of a rotating cylinder. In an example, a plurality of extendable fins can together comprise an airfoil structure when they are extended outwards from the walls of a rotating cylinder. In an example, a plurality of extendable fins can together comprise a fluid propeller structure when they are extended outwards from the walls of a rotating cylinder.

In the example in FIGS. 89 through 91, implanted blood flow lumen 9002 has been endovascularly and/or transluminally inserted and expanded inside the walls of blood vessel 8901. In this example, implanted blood flow lumen 9002 comprises a substantially-cylindrical structure. In an example, implanted blood flow lumen 9002 can be like a stent, except that it has a more complex structure which includes rotating cylinder 9001 and extendable fins 9005, 9006, 9007, and 9008. In this example, rotating cylinder 9001 rotates in a coaxial manner within implanted blood flow lumen 9002. In an example, rotating cylinder 9001 can be rotated by magnetic interaction with an electromagnetic field which is generated by control units 9003 and 9004. In another example, rotating cylinder 9001 can be rotated by a direct mechanical drive mechanism which operated by control units 9003 and 9004.

In a example, a rotating cylinder can rotate along bearings, tracks, or grooves which are part of implanted blood flow lumen 9002. In an example, implanted blood flow lumen 9002 and rotating cylinder 9001 can be inserted and expanded together as a single connected unit. In an example, implanted blood flow lumen 9002 and rotating cylinder 9001 can be inserted and expanded separately, as different pieces, but they can be connected together in vivo. In an example, implanted blood flow lumen 9003 and rotating cylinder 9001 can be connected prior to implantation. In an example, they can be connected in vivo.

In an example, extendable fins 9005, 9006, 9007, and 9008 can each have one portion (such as a side or end) which is attached to a wall of rotating cylinder 9001 and one portion (such as a side or end) which is not attached. In an example, the unattached portion of an extendable fin is free to bend or extend outwards from the cylinder wall into the central area of the implanted blood flow lumen. In an example, an extendable fin can have a shape memory such that its unattached portion has a natural disposition (absent external force) to remain flush against the wall of the rotating cylinder. In an example, an unattached portion of an extendable fin can be induced to bend or extend into the central area of the implanted blood flow lumen by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material. In this example, an unattached portion of an extendable fin is induced to bend or extend into the central area of the implanted blood flow lumen by frictional engagement with blood as the rotating cylinder begins to rotate. In this example, an unattached portion of an extendable fin will naturally return (due to its shape memory) to a flush position against the cylinder wall when the cylinder stops rotating.

In an example, extendable fins 9005, 9006, 9007, and 9008 can have a first (retracted) configuration wherein they are retracted and be relatively flush with the walls of rotating cylinder 9001. In an example, extendable fins 9005, 9006, 9007, and 9008 have a second (protruding) configuration wherein they are extended outward from the sides of cylinder 9001 toward the center of implanted blood flow lumen 9002. In an example, extendable fins can be moved from the first configuration to the second configuration as a blood flow increasing mechanism starts to operate. In an example, these fins can protrude in a second configuration so as to frictionally engage blood and increase blood flow when the blood flow increasing mechanism is in operation. In an example, these fins can retract so as to be flush against the cylinder wall and not hinder native blood flow when the blood flow increasing mechanism is not in operation.

In an example, extendable fins 9005, 9006, 9007, and 9008 can be configured to move from the first configuration to the second configuration due to friction with blood when the cylinder begins to rotate. In this manner, when the cylinder begins to rotate, the fins are automatically pulled outwards by friction with blood. In this example, the extendable fins can automatically retract back toward the cylinder walls due to material shape memory and/or a spring mechanism when the cylinder stops rotating. In another example, extendable fins can be extended or retracted by an electromagnetic field that is generated by the control units. In another example, extendable fins can be extended or retracted by microscale actuators. In another example, extendable fins can be extended or retraced by centripetal/fugal force. [There really is no such thing as “centrifugal force,” but the term is colloquially used to describe “centripetal force” so I fudge a bit by including both terms.] With any of these methods, this device increases blood flow when the blood flow increasing mechanism is in operation, but does not hinder native blood flow when the blood flow increasing mechanism is not in operation. In this example, extendable projecting members within the rotating cylinder are specified as fins. In other examples, one or more extendable projecting members within a rotating cylinder can be selected from the group consisting of: fins, vanes, blades, winglets, airfoils, and helical structures.

FIGS. 89 through 91 show an example of how this invention can be embodied in a device wherein a blood flow increasing mechanism has a first configuration (with retracted fins) when it is not in operation transducing electromagnetic energy into kinetic energy, wherein the blood flow increasing mechanism has a second configuration (with extended fins) when it is in operation transducing electromagnetic energy into kinetic energy, and wherein the second configuration occupies a larger portion of the post-implantation minimum cross-sectional flow area than the first configuration. FIGS. 89 through 91 also show an example of how this invention can be embodied in a device wherein the blood flow increasing mechanism is moved from the first configuration (retracted fins) to the second configuration (extended fins) by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

In an example, control units 9003 and 9004 can control the rotation of rotating cylinder 9001 (and the extension of fins 9005, 9006, 9007, and 9008) based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control units 9003 and 9004 can control the rotation of rotating cylinder 9001 (and the extension of fins 9005, 9006, 9007, and 9008) based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit.

FIGS. 92 through 95 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

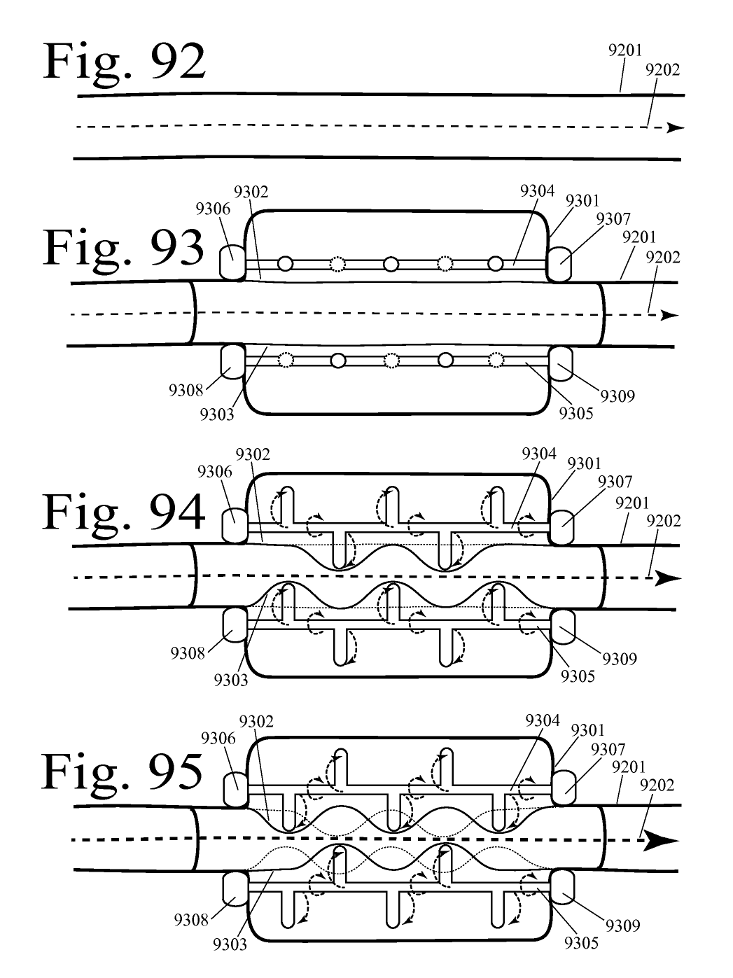

FIG. 92 shows a blood vessel before implantation. FIG. 93 shows a longitudinal semi-transparent view of the device (after implantation) at a time when the blood flow increasing mechanism is not in operation. FIG. 94 shows a longitudinal semi-transparent view of the device at a time when the blood flow increasing mechanism is in operation, during a first cycle phase. FIG. 95 shows a longitudinal semi-transparent view of the device at a time when the blood flow increasing mechanism is in operation, during a second cycle phase. FIGS. 92 through 95 show: blood vessel 9201, blood flow 9202, implanted blood flow lumen 9301, first flexible membrane 9302, second flexible membrane 9303, first crankshaft-like member 9304, second crankshaft-like member 9305, and control units 9306, 9307, 9308, and 9309. Control units can further comprise one or more power sources, actuators, and wireless data transmitters/receivers.

In this example, implanted blood flow lumen 9301 is spliced into blood vessel 9201 so as to entirely replace a longitudinal segment of the blood vessel. In this example, implanted blood flow lumen 9301 has an arcuate non-uniform cross-sectional shape. In this example, implanted blood flow lumen 9301 has a larger central cross-sectional area, but the portions which house the crankshaft-like members are separated from fluid communication with blood by the flexible membranes. In this example, control units 9306, 9307, 9308, and 9309 rotate crankshaft-like members 9304 and 9305. This rotation causes moving protrusions on these crankshaft-like members to come into alternating contact with flexible membranes 9302 and 9303. This alternating contact propagates a longitudinal (upstream to downstream) wave motion along these membranes. This longitudinal wave motion frictionally engages blood which increases blood flow through the implanted blood flow lumen.

In this example, the protrusions on the crankshaft-like members which engage the flexible membranes are smooth and arcuate so that they do not tear the flexible membranes as they come into repeated contact. In this example, the two crankshaft-like members have similarly sized and spaced protrusions. In an example, the two crankshaft-like members can have differently sized or spaced protrusions. In an example, the two crankshaft-like members can rotate in phase with each other. In an example, the two crankshaft members can rotate out of phase with each other. In an example, there can be more than two crankshaft-like members and more than two flexible membranes in contact with blood. In an example, the combined motion of the two flexible membranes, 9302 and 9303, in this design can comprise peristaltic motion. However, depending on the relative shapes, motion phases, and motion speeds of the two crankshaft-like members, this design can produce blood flow inducing motions which are more general than classic peristaltic motion.

There are potential advantages to this design. As shown in FIG. 93, flexible membranes 9302 and 9303 (which comprise the lumen walls in a central portion of the lumen) are substantially flat and smooth when the blood flow increasing mechanism is not in operation (when the crankshaft-like members are in the position shown in FIG. 93). This can help to minimize thrombogenesis. Also, as shown in FIG. 93, flexible membranes 9302 and 9303 (which comprise the lumen walls in a central portion of the lumen) do not intrude into the center of the lumen when the blood flow increasing mechanism is not in operation (when the crankshaft-like members are in the position shown in FIG. 93). This can allow unhindered native blood flow when the blood flow increasing mechanism is not in operation.

FIG. 93 shows this device at a time when the blood flow increasing mechanism is not in operation. At this time, the two crankshaft-like members, 9304 and 9305, are rotated into (neutral) positions wherein their protrusions do not engage the two flexible membranes 9302 and 9303. In this configuration, the membranes are flat and smooth and do not intrude into the central cross-sectional blood flow area of the implanted blood flow lumen. This allows unhindered native blood flow.

FIG. 94 shows this device at another time, wherein the blood flow increasing mechanism is in operation in a first phase cycle. At this time, the two crankshaft-like members, 9304 and 9305, are rotated into positions wherein their protrusions engage the two flexible membranes 9302 and 9303. In this configuration, the membranes are moved into first sinusoidal-shaped wave configurations which intrude into the central cross-sectional blood flow area of the implanted blood flow lumen.

FIG. 95 shows this device at another time, wherein the blood flow increasing mechanism is in operation in a second phase cycle. At this time, the two crankshaft-like members, 9304 and 9305, are rotated into positions wherein their protrusions engage the two flexible membranes 9302 and 9303. In this configuration, the membranes are moved into second sinusoidal-shaped wave configurations which intrude into the central cross-sectional blood flow area of an implanted blood flow lumen. In FIG. 95, the (prior) first sinusoidal-shaped wave configurations from FIG. 94 is displayed as dotted lines to highlight the change from the first shape to the second shape from FIG. 94 to FIG. 95. In this example, the sequential movement of the flexible membranes from the first and second sinusoidal-shaped wave configurations acts to increase blood flow through the implanted blood flow lumen.

In an example, control units 9306, 9307, 9308, and 9309 can control the rotation of crankshaft-like members 9304 and 9305 based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control units 9306, 9307, 9308, and 9309 can control the rotation of crankshaft-like members 9304 and 9305 based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit.

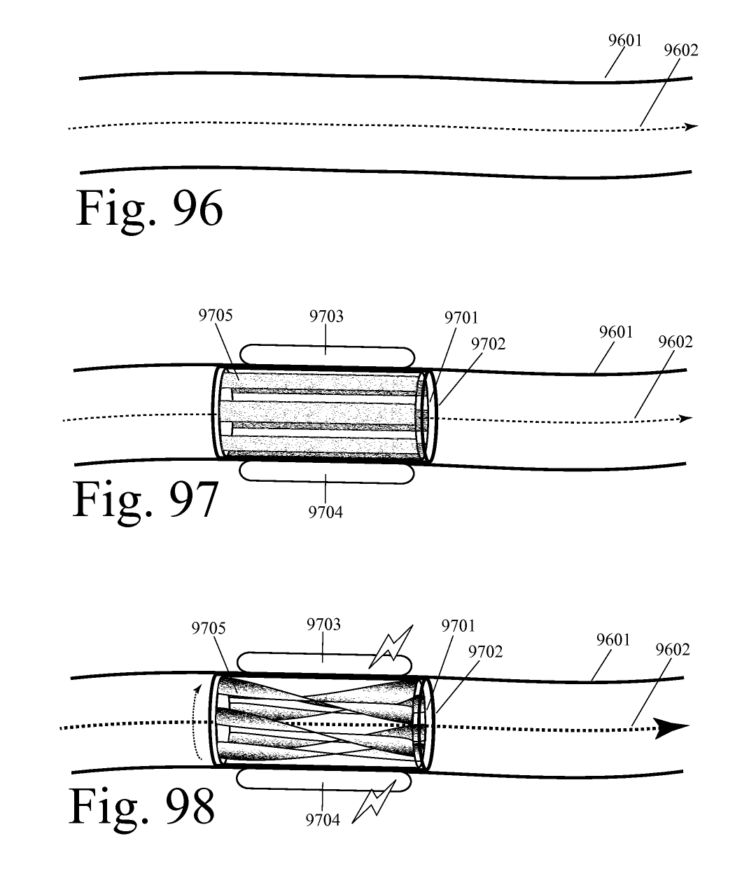

FIGS. 96 through 98 show another example of how this invention can be embodied in an implanted extracardiac device for supplementing blood circulation comprising: (a) at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; (b) a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and (c) a control unit for the blood flow increasing mechanism.

FIG. 96 shows a blood vessel before implantation. FIG. 97 shows a longitudinal semi-transparent view of the device (after implantation) at a time when the blood flow increasing mechanism is not in operation. FIG. 98 shows a longitudinal semi-transparent view of the device at a time when the blood flow increasing mechanism is in operation. FIGS. 96 through 98 show: blood vessel 9601, blood flow 9602, implanted blood flow lumen 9702, rotating member 9701, twistable strips (including 9705), and control units 9703 and 9704. In an example, the control units can further comprise a power source, an electromagnetic field generator, and a wireless data transmitter/receiver. In an example, twistable fins, vanes, blades, airfoils, winglets, or helical structures can be used instead of twistable strips. In an example, a plurality of twistable strips, fins, vanes, blades, airfoils, winglets, or helical structures can comprise an impeller when they are in a twisted configuration.

In this example, implanted blood flow lumen 9702 has been endovascularly and/or transluminally inserted and expanded inside the walls of blood vessel 9601. In this example, implanted blood flow lumen 9702 has a substantially-cylindrical structure. In an example, implanted blood flow lumen 9702 can be like a stent, except that it has a more complex structure which includes rotating member 9701 and twistable strips (including 9705). In this example, rotating member 9701 rotates within implanted blood flow lumen 9702. In this example, rotating member 9701 is rotated by magnetic interaction with an electromagnetic field which is generated by control units 9703 and 9704. In an alternative example, rotating member 9701 can be rotated by a direct mechanical drive mechanism. In an example, rotating member 9701 can rotate on bearings, tracks, or grooves which are part of implanted blood flow lumen 9702.

In an example, implanted blood flow lumen 9702 and rotating member 9701 can be connected prior to implantation. In an example, implanted blood flow lumen 9702 and rotating member 9701 can be inserted and expanded at the same time. In an example, implanted blood flow lumen 9702 and rotating member 9701 can be inserted and expanded at different times. In an example, implanted blood flow lumen 9702 and rotating member 9701 can be connected in vivo.

In an example, rotating member 9701 can further comprise two bands (or rings) to which twistable strips (including 9705) are attached. In an example, each of the twistable strips (including 9705) can have one portion (such as an end or side) which is attached to an upstream band and one portion (such as an end or side) which is attached to a downstream band. In an example, an upstream band and a downstream band can be rotated in manners which are at least partially independent from each other. In an example, when an upstream band is partially rotated relative to an downstream band, then this twists the twistable strips. In an example, when the twistable strips are twisted, then they collectively form an impeller within implanted blood lumen 9702. In an example, when the twistable strips are in a twisted configuration and rotating member 9701 rotates, this increases blood flow through implanted blood flow lumen 9702.

FIG. 97 shows this device in a first configuration in which an upstream band and a downstream band are in rotational alignment. In this configuration, the twistable strips (including 9705) are not twisted. In this first configuration, the twistable strips (including 9705) are longitudinally straight and are flush against the walls of rotating member 9701. In this first configuration, the twistable strips do not substantially block the cross-sectional flow area through implanted blood flow lumen 9702 and do not hinder native blood flow.

FIG. 98 shows this device in a second configuration in which an upstream band and a downstream band are not in rotational alignment. In this configuration, the twistable strips (including 9705) are twisted. In this second configuration, the twistable strips (including 9705) collectively comprise an impeller structure. In an example, this impeller structure can be helical. In this second configuration, the twistable strips block the cross-sectional flow area through implanted blood flow lumen 9702, but they increase blood flow when rotating member 9701 is rotated.

In an example, this device can be transitioned from the first configuration to the second configuration before or as the blood flow increasing mechanism begins operate. In an example, this device is transitioned from the first configuration to the second configuration by differential rotation of an upstream band and a downstream band, wherein a plurality of twistable strips are connected at different ends to these two bands. In an example, differential rotation of an upstream band versus a downstream band can occur due to inertia whenever rotating member 9701 begins to rotate. In an example, differential rotation of an upstream band versus a downstream band can be controlled separately from the rotation of member 9701. In an example, a plurality of twistable strips can be moved from a first (untwisted) configuration to a second (twisted) configuration by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

This design has potential advantages. First, a large portion of the device can be implanted within the walls of the blood vessel and thus can be implanted in a minimally invasive manner. Second, the twistable strips enable the device to frictionally engage and increase blood flow when the blood flow increasing member is in operation, but not hinder native blood flow when the blood flow increasing member is not in operation. Third, if the twistable strips can be held sufficiently flush to the lumen walls when the blood flow increasing member is not in operation, then this can create a smooth wall surface which can minimize thrombogenesis.

FIGS. 96 through 98 show an example of how this invention can be embodied in a device wherein the blood flow increasing mechanism has a first configuration (untwisted strips) when it is not in operation transducing electromagnetic energy into kinetic energy, wherein the blood flow increasing mechanism has a second configuration (twisted strips) when it is in operation transducing electromagnetic energy into kinetic energy, and wherein the second configuration occupies a larger portion of the post-implantation minimum cross-sectional flow area than the first configuration. FIGS. 96 through 98 also show an example of how this invention can be embodied in a device wherein the blood flow increasing mechanism is moved from the first configuration to the second configuration by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

In an example, control units 9703 and 9704 can control the rotation of cylinder 9701 (and the twisting of strips including 9705) based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In an example, control units 9703 and 9704 can control the rotation of cylinder 9701 (and the twisting of strips including 9705) based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In an example, this device can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In an example, a plurality of such circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit. In an example, this invention can be embodied in a method for distributed, adjustable, coordinated, and therapeutic extracardiac circulatory flow assistance which can helps to avoid cardiac function deterioration and/or facilitate cardiac function recovery. In an example, this method can provide maximum benefit to those body organs which are in the greatest need. In an example, this method can involve functional coordination among a distributed system of devices in order to achieve maximum therapeutic benefit.

FIGS. 1 through 98 have shown examples of how this invention can be embodied in an implanted extracardiac device for supplementing blood circulation comprising: at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location; a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and a control unit for the blood flow increasing mechanism.

FIGS. 1 through 98 have also shown examples of how this invention can be embodied in a device wherein: a pre-implantation minimum cross-sectional flow area is the minimum cross-sectional flow area from the upstream location to the downstream location before the implanted blood flow lumen and the blood flow increasing mechanism are implanted; wherein a post-implantation minimum cross-sectional flow area is the minimum cross-sectional flow area from the upstream location to the downstream location which is unobstructed by the blood flow increasing mechanism when the blood flow increasing mechanism is not in operation after the implanted blood flow lumen and the blood flow increasing mechanism are implanted; and wherein the post-implantation minimum cross-sectional flow area is not substantially less than the pre-implantation minimum cross-sectional flow area. These figures have also shown examples wherein the definition of substantially less can be selected from the group consisting of: 5% less, 10% less, and 25% less.

FIGS. 1 through 98 have also shown examples of how post-implantation blood flow from an upstream location to a downstream location can be greater than pre-implantation blood flow from the upstream location to the downstream location when a blood flow increasing mechanism is in operation (transducing electromagnetic energy into kinetic energy) while post-implantation blood flow from the upstream location to the downstream location when the blood flow increasing mechanism is not in operation is not substantially less than pre-implantation blood flow from the upstream location to the downstream location

FIGS. 1 through 98 have also shown examples of how a blood flow lumen of this device can be implanted entirely within a blood vessel, implanted at least partially outside a blood vessel, or implanted so as to completely replace a longitudinal section of a blood vessel. FIGS. 1 through 98 have also shown examples of how a post-implantation minimum cross-sectional flow area can comprise the combined cross-sectional area through which blood flows unobstructed from the upstream location to the downstream location through either the implanted blood flow lumen or the blood vessel with which it is in fluid communication.

In various examples, including those shown in FIGS. 1 through 98, an implanted blood flow lumen can be implanted into fluid communication with a blood vessel by one or more connecting members or connection methods which are selected from the group consisting of: endovascular insertion and expansion within a blood vessel, anastomosis, sutures, purse string suture, drawstring, pull tie, friction fit, surgical staples, tissue adhesive, gel, fluid seal, biochemical bond, cauterization, (three-way) vessel joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

In various examples, including those shown in FIGS. 1 through 98, an implanted blood flow lumen can be selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber.

In various examples, including those shown in FIGS. 1 through 98, a blood flow increasing mechanism can be selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump.

As shown in FIGS. 1 through 98, a blood flow increasing mechanism can have a first configuration when it is not in operation transducing electromagnetic energy into kinetic energy and can have a second configuration when it is in operation transducing electromagnetic energy into kinetic energy. Further, the second configuration can occupy a larger portion of the post-implantation minimum cross-sectional flow area than the first configuration. Further, the post-implantation minimum cross-sectional flow area can be substantially less than the pre-implantation minimum cross-sectional flow area when the blood flow increasing mechanism is in the second configuration, but not when the blood flow increasing mechanism is in the first configuration. In various examples, including those in FIGS. 1 through 98, a blood flow increasing mechanism can be moved from the first configuration to the second configuration by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

In various examples, including those in FIGS. 1 through 98, a control unit for a blood flow increasing mechanism can change the operation of the blood flow increasing mechanism based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

In various examples, including those in FIGS. 1 through 98, a control unit for the blood flow increasing mechanism can change the operation of the blood flow increasing mechanism based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

In various examples, including those in FIGS. 1 through 98, this invention can further comprise one or more additional components selected from the group consisting of: a power source and/or power transducer, an electric motor, a data processing unit, a digital memory, a wireless data receiver and/or transmitter, a (one-way) fluid valve, an implanted sensor, a (deployable) thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

In various examples, including those in FIGS. 1 through 98, a plurality of circulatory assistance devices can be implanted in multiple selected extracardiac locations within a person's circulatory system in order to create a distributed, adjustable, coordinated, and therapeutic system of extracardiac circulatory flow assistance which helps to avoid cardiac function deterioration and/or facilitate cardiac function recovery. In an example, the functions of such devices distributed throughout selected locations in a person's circulatory system can be coordinated so as to provide maximum benefit to those body organs which are in the greatest need. In an example, the functions of devices distributed throughout selected locations in a person's circulatory system can be coordinated in order to achieve maximum therapeutic benefit. In an example, this invention can be embodied in a method for distributed, adjustable, coordinated, and therapeutic extracardiac circulatory flow assistance which can helps to avoid cardiac function deterioration and/or facilitate cardiac function recovery. In an example, this method can provide maximum benefit to those body organs which are in the greatest need. In an example, this invention can be embodied in a system comprising a plurality of the devices disclosed in FIGS. 1 through 98 which are implanted in selected extracardiac locations within a person's circulatory system wherein the functions of these devices are coordinated in order to help to avoid cardiac function deterioration and/or facilitate cardiac function recovery.

I claim:

1. An implanted extracardiac device for supplementing blood circulation comprising:
at least one implanted blood flow lumen, wherein this implanted blood flow lumen is configured to be implanted within a person's body so as to receive blood inflow from a blood vessel at an upstream location with respect to the natural direction of blood flow, wherein this implanted blood flow lumen is configured to discharge blood into a blood vessel at a downstream location with respect to the natural direction of blood flow, wherein this implanted blood flow lumen has a longitudinal axis spanning from the upstream location to the downstream location, wherein this implanted blood flow lumen has a cross-sectional area through which blood can flow which is substantially perpendicular to the longitudinal axis, and wherein a minimum cross-sectional flow area is defined as the minimum unobstructed cross-sectional area through which can blood flow from the upstream location to the downstream location;
a blood flow increasing mechanism, wherein this blood flow increasing mechanism is configured to be implanted within a person's body, wherein this blood flow increasing mechanism is configured to increase the flow of blood from the upstream location to the downstream location when the blood flow increasing mechanism is in operation by transducing electromagnetic energy into kinetic energy; and
a control unit for the blood flow increasing mechanism.

2. The device in claim 1 wherein: a pre-implantation minimum cross-sectional flow area is the minimum cross-sectional flow area from the upstream location to the downstream location before the implanted blood flow lumen and the blood flow increasing mechanism are implanted; wherein a post-implantation minimum cross-sectional flow area is the minimum cross-sectional flow area from the upstream location to the downstream location which is unobstructed by the blood flow increasing mechanism when the blood flow increasing mechanism is not in operation after the implanted blood flow lumen and the blood flow increasing mechanism are implanted; and wherein the post-implantation minimum cross-sectional flow area is not substantially less than the pre-implantation minimum cross-sectional flow area.

3. The device in claim 2 wherein substantially less is 5% less.

4. The device in claim 2 wherein substantially less is 10% less.

5. The device in claim 2 wherein substantially less is 25% less.

6. The device in claim 1 wherein: post-implantation blood flow from the upstream location to the downstream location is greater than pre-implantation blood flow from the upstream location to the downstream location when the blood flow increasing mechanism is in operation transducing electromagnetic energy into kinetic energy; and wherein post-implantation blood flow from the upstream location to the downstream location when the blood flow increasing mechanism is not in operation is not substantially less than pre-implantation blood flow from the upstream location to the downstream location

7. The device in claim 1 wherein the implanted blood flow lumen is configured to be implanted entirely within a blood vessel.

8. The device in claim 1 wherein the implanted blood flow lumen is configured to be implanted at least partially outside a blood vessel.

9. The device in claim 1 wherein the implanted blood flow lumen is configured to replace a longitudinal section of a blood vessel.

10. The device in claim 1 wherein the post-implantation minimum cross-sectional flow area comprises the combined cross-sectional area through which blood flows unobstructed from the upstream location to the downstream location through either the implanted blood flow lumen or the blood vessel with which it is in fluid communication.

11. The device in claim 1 wherein the implanted blood flow lumen is configured to be implanted into fluid communication with a blood vessel by one or more connecting members or connection methods which are selected from the group consisting of: endovascular insertion and expansion within a blood vessel, anastomosis, sutures, purse string suture, drawstring, pull tie, friction fit, surgical staples, tissue adhesive, gel, fluid seal, biochemical bond, cauterization, (three-way) vessel joint, vessel branch, twist connector, helical threads or screw connector, connection port, interlocking joints, tongue and groove connection, flanged connector, beveled ridge, magnetic connection, plug connector, circumferential ring, inflatable ring, and snap connector.

12. The device in claim 1 wherein the implanted blood flow lumen is selected from the group consisting of: artificial vessel segment, bioengineered vessel segment, transplanted vessel segment, artificial vessel joint, vessel branch, stent or other expandable mesh or framework, artificial lumen, manufactured catheter, manufactured tube, valve, vessel valve segment, multi-channel lumen, blood pump housing, and elastic blood chamber.

13. The device in claim 1 wherein the blood flow increasing mechanism is selected from the group consisting of: Archimedes pump, axial pump, balloon pump, biochemical pump, centripetal/fugal pump, ciliary motion pump, compressive pump, continuous flow pump, diaphragm pump, elastomeric pump, electromagnetic field pump, electromechanical pump, electroosmotic pump, extracardiac pump, gear pump, hybrid pulsatile and continuous pump, hydrodynamically-levitated pump, hydroelastic pump, impedance pump, longitudinal-membrane-wave pump, magnetic flux pump, Micro Electro Mechanical System (MEMS) pump, native flow entrainment pump, peripheral vasculature pump, peristaltic pump, piston pump, pulsatile flow pump, pump that moves fluid by direction interaction between fluid and an electromagnetic field, pump with a helical impeller, pump with a parallel-axis impeller, pump with a perpendicular-axis impeller, pump with a series of circumferentially-compressive members, pump with an expansion chamber and one-way valve, pump with an impeller with multiple vans, fins, and/or blades, pump with electromagnetically-driven magnetic impeller, pump with fluid jets which entrain native blood flow, pump with helical impeller, pump with magnetic bearings, pump with reversibly-expandable impeller projections, rotary pump, sub-cardiac pump, and worm pump.

14. The device in claim 1 wherein the blood flow increasing mechanism has a first configuration when it is not in operation transducing electromagnetic energy into kinetic energy, wherein the blood flow increasing mechanism has a second configuration when it is in operation transducing electromagnetic energy into kinetic energy, and wherein the second configuration occupies a larger portion of the post-implantation minimum cross-sectional flow area than the first configuration.

15. The device in claim 14 wherein the post-implantation minimum cross-sectional flow area is substantially less than the pre-implantation minimum cross-sectional flow area when the blood flow increasing mechanism is in the second configuration, but not when the blood flow increasing mechanism is in the first configuration.

16. The device in claim 14 wherein the blood flow increasing mechanism is moved from the first configuration to the second configuration by one or more means selected from the group consisting of: centripetal/fugal force, differential rotational an upstream member and a downstream member, electromagnetic force, fluid resistance and/or frictional engagement, hydraulic force, inflation and/or pneumatic force, MEMS or other microscale actuation, piezoelectric effect, and reversible shape memory material.

17. The device in claim 1 wherein the control unit for the blood flow increasing mechanism changes the operation of the blood flow increasing mechanism based on one or more factors selected from the group consisting of: bioimpedance, blood oxygen saturation, blood pressure or pressure differentials, blood viscosity level, brain oxygenation, cardiac function parameters, cardiac performance, cardiac wall stress, clot and/or thrombus detection, data from a pacemaker or defibrillator, ECG data and/or patterns, edema in downstream veins, EEG data and/or patterns, ejection fraction, electrical power availability, electrical power stored, EMG data and/or patterns, exercise and/or body movement, heart performance, heart sounds, heart vibration, heart workload, hemodynamics, impeller rotational resistance, infection detection, local/body power harvesting opportunities, non-cardiac organ function, one or more blood flow rates, pulse oximetry, pulse rate, pump performance, secure input from a health care provider, temperature, thrombogenic conditions, tissue oxygenation, vessel elasticity, and wash cycle to reduce thrombogenesis.

18. The device in claim 1 wherein the control unit for the blood flow increasing mechanism changes the operation of the blood flow increasing mechanism based on data received from one or more sensors selected from the group consisting of: acoustic sensor, barometer, biochemical sensor, blood flow rate sensor, blood glucose sensor, blood oximetry sensor, blood pressure sensor, blood viscosity sensor, brain oxygen level sensor, capnography sensor, cardiac function sensor, cardiotachometer, chewing and/or swallowing sensor, chromatography sensor, clot and/or thrombus sensor, coagulation sensor, cutaneous oxygen sensor, digital stethoscope, Doppler ultrasound sensor, ear oximeter, ejection fraction sensor, electrocardiogram (ECG) monitor or sensor, electroencephalography (EEG) monitor or sensor, electrogastrography (EGG) sensor and/or monitor, electromagnetic conductivity sensor, electromagnetic impedance sensor, electromagnetic sensor, electromyography (EMG) monitor or sensor, electroosmotic sensor, flow rate sensor, fluid flow sensor, food consumption sensor, gastric function sensor, global positioning system (GPS) module, glucose sensor, goniometer, gyroscope, heart acoustics sensor, heart rate sensor, heart vibration sensor, hemoencephalography (HEG) sensor, hydration sensor, impedance sensor, inertial sensor, infrared sensor, magnetic field sensor, magnometer, microbial sensor, Micro-Electro-Mechanical System (MEMS) sensor, microfluidic sensor, motion sensor and/or multi-axial accelerometer, neural impulse sensor, oximetry sensor, oxygen consumption sensor, oxygen saturation monitor, pH level sensor, photoplethysmography (PPG) sensor, piezoelectric sensor, pneumography sensor, pressure or flow sensor, pressure sensor, pulmonary and/or respiratory function sensor, pulse sensor, renal function sensor, rotational speed sensor, spectral analysis sensor, spectroscopy sensor, stretch sensor, thermal energy sensor, thrombus sensor, torque sensor, ultrasonic sensor, ultraviolet sensor, and viscosity sensor.

19. The device in claim 1 wherein this invention further comprises one or more additional components selected from the group consisting of: a power source and/or power transducer, an electric motor, a data processing unit, a digital memory, a wireless data receiver and/or transmitter, a one-way fluid valve, an implanted sensor, a deployable thrombus catching net or mesh, a drug reservoir and/or pump, a MEMS actuator, a radioopaque marker, a wearable sensor with which the device is in wireless communication, a blood reservoir, a magnetic field generator, an electromagnetic energy emitter, a computer-to-human interface, and a human-to-computer interface.

20. A system comprising a plurality of the devices in claim 1 which are implanted in selected extracardiac locations within a person's circulatory system wherein the functions of these devices are coordinated in order to help to avoid cardiac function deterioration and/or facilitate cardiac function recovery.
